(19) United States
(12) Patent Application Publication
(10) Pub. No.: US 2013/0100402 A1
(43) Pub. Date: Apr. 25, 2013

(54) SYSTEMS AND METHODS FOR REDUCING SENSORY EYE DOMINANCE

(71) Applicants: UNIVERSITY OF LOUISVILLE, Louisville, KY (US); SALUS UNIVERSITY, Elkins Park, PA (US)

(72) Inventors: TENG LENG OOI, Wyncote, PA (US); ZIJIANG J. HE, Louisville, KY (US)

(73) Assignees: SALUS UNIVERSITY, Elkins Park, PA (US); UNIVERSITY OF LOUISVILLE, Louisville, KY (US)

(21) Appl. No.: 13/650,564

(22) Filed: Oct. 12, 2012

Related U.S. Application Data
(60) Provisional application No. 61/546,186, filed on Oct. 12, 2011.

(51) Int. Cl. A61B 3/10

(52) U.S. Cl. CPC A61B 3/10
USPC 351/203; 351/246

(57) ABSTRACT

The present invention relates to systems and methods for providing a push/pull perceptual learning technique to a subject demonstrating sensory eye dominance (SED) and/or amblyopia. More specifically, the weak eye of the subject is cued forcing it to become dominant, while visualization in the strong eye is suppressed over the course of a treatment regimen. Such systems and methods are shown herein to result in a perceptual learning and a reduction of interocular imbalance, as well as an improvement in the visual characteristics typically associated with very little or no SED and/or amblyopia, such as improved depth perception.

### CROSS REFERENCE TO RELATED APPLICATIONS

The present application claims priority to U.S. Provisional Application Ser. No. 61/546,186, filed on Oct. 12, 2011, the contents of which are incorporated herein by reference in its entirety.

This invention was made with Government support under RO1 EY 015804 awarded by the National Institutes of Health. The Government has certain rights in this invention.

### FIELD OF THE INVENTION

The present invention relates to systems and methods for reducing binocular imbalance, sensory eye dominance, and/or amblyopia in a subject by stimulating the subject's weak or non-dominant eye and inhibiting the strong or dominant eye.

### BACKGROUND OF THE INVENTION

Binocular vision contributes to the visual ability of figure-ground segmentation and fine depth discrimination. Retinal images of 3-D visual scenes from the two eyes usually have the same mean contrast energy over time. This suggests that the binocular visual system is built to treat the inputs from the two eyes equally in order to achieve a high proficiency. Indeed, for a standard observer, stimuli with equal contrast in each eye induces superior binocular perception, as compared to stimuli with unequal contrast levels.

The interocular integration and inhibitory mechanisms that are part of the binocular neural network support a variety of binocular visual functions including summation, fusion, stereopsis and suppression. Both mechanisms work together, with the interocular inhibitory mechanism suppressing dissimilar images from one or both eyes, to achieve a coherent 3-D representation of the visual scene. Binocular visual processing is adversely affected, however, when an observer's eyes are not equally strong, i.e. one eye is dominant over the other and provides a larger weighted contribution to the binocular neural network. Indeed, human observers with a significant degree of unbalanced interocular inhibition, called sensory eye dominance (SED), tend to have degraded binocular visual processing and reduced binocular depth perception.

FIG. 1a shows a simplified framework to conceptually understand the putative excitatory and inhibitory networks. Inputs of hypothetical cortical units with common orientation preference from the two eyes converge while monocular units with preference for orthogonal orientation inhibit one another. The mutual inhibition between inputs of the two eyes is largely balanced in the normally developed adult. But when the mutual inhibition is unbalanced, resulting in SED, binocular vision is degraded. FIG. 1b conceptualizes an example where the right eye's (RE) inhibition on the left eye (LE) is stronger. When stimulated with dichoptic orthogonal gratings of the same contrast to induce binocular competition, signals in the LE's channel are suppressed while signals in the RE's channel travel upstream leading to its image being perceived.

The magnitude of SED varies in the population along a continuum. At one end, observers with minor SED have clinically normal stereoacuity. At the other end, however, observers with strong SED have little or no stereopsis. One example of the latter is a condition called amblyopia (or “lazy eye”), which is also characterizable by a host of visual deficits related to contour integration, spatial and temporal vision, as well as those related to higher level visual functions.

Based on the foregoing, there is a continuing need for establishing treatment methods and regimens that can correct SED and/or amblyopia. In particular, there is a need for a system, method, or protocol, that can reduce and/or correct binocular imbalance in a subject and improve the visual impairments associated with the dominance of one eye of a subject over the other. The present invention addresses at least this need.

### SUMMARY OF THE INVENTION

The present invention relates to systems and methods for reducing sensory eye dominance, interocular imbalance, and/or amblyopia in a subject by providing to the subject's non-dominant eye and dominant eye separate non-identical binocular rivalry visual stimuli, where the visual stimulus presented to the non-dominant eye is preferred by the observer over that presented to the dominant eye such that the visual attention of the observer remains with the non-dominant eye during the presentation.

In one non-limiting embodiment, the method includes providing a first visual stimulus to at least a foveal visual region of a non-dominant eye of a subject and a second visual stimulus to at least a foveal region of a dominant eye of the subject, wherein visualization of the visual stimulus in the non-dominant eye is enabled through neural excitation and visualization of the visual stimulus in the dominant eye is inhibited. These stimuli may be provided as one or more pairs of dichoptic grating discs, where an orientation of the second visual stimulus is at an angle from about 0 to about 180 degrees, relative to an orientation the first visual stimulus, and in certain embodiments, the two grating discs are orthogonally oriented. Such discs may be provided against a blank background or, alternatively, against a grating background. When presented against the latter, the first visual stimulus is provided at an angle that is greater than about 0 degrees to less than about 180 degrees to the grating background, and in certain preferred embodiments is orthogonal to the grating background. The second visual stimulus is approximately parallel to the grating background and may be phase shifted from the grating background between about 0 degrees to about 180 degrees. In addition to stimulus presentation in the foveal region, in further embodiments, one or more additional stimuli may also or alternatively be presented in a parafoveal visual region of the subject in accordance with the foregoing.

Selection of or visualization of the visual stimulus presented to the non-dominant eye over the visual stimulus presented to the dominant eye may be initiated using an attention cue. The attention cue may be provided as a separate visualization element, such as a rectangular frame, that is presented before the visual stimulus is presented. In alternative embodiments, however, the visual stimulus, itself, may serve to shift the subject's focus to the non-dominant eye, particularly, though not exclusively, when the stimulus in the non-dominant eye has a high saliency. Thus, in such an embodiment, a secondary attention cue is unnecessary. Specifically, in place of the attention cue, any pair of binocular rivalry stimulus will suffice to focus attention to the non-dominant eye, so long as the stimulus in the non-dominant eye has a higher stimulus strength.

In further embodiments of the present invention, successive sets of visual stimulation, such as those provided above or otherwise herein, are presented to both the dominant and non-dominant eyes wherein physical characteristics of the stimuli between at least two or more sets are changed. For example, in one aspect, a first set of separate visual stimuli are provided to a non-dominant eye and dominant eye of a subject, wherein visualization of the visual stimuli in the non-dominant eye is selected over visualization of the visual stimulus in the dominant eye. The first set of separate non-identical visual stimuli is removed and a second set of separate non-identical visual stimuli is provided, where visualization of the visual stimulus in the non-dominant eye is again selected over visualization of the second set of visual stimulus in the dominant eye. At least one physical characteristic of the visual stimuli presented to the non-dominant eye is different than that of the first set of visual stimuli.

The changed physical characteristic may be selected from any one or more of a change of stimuli orientation angle, a change of stimuli contrast, and/or the addition of one or more visual enhancement features. By way of example, in certain embodiments, the orientation angle of the second visual stimulus presented to the non-dominant eye is different than the angle of the first visual stimulus presented to the non-dominant eye by an amount that is greater than about 0 degrees but less than about 180 degrees. In other aspects, the contrast of the second visual stimulus presented to the non-dominant eye is higher or lower than the contrast of the first visual stimulus presented to the non-dominant eye.

In even further embodiments, the visual stimuli provided to the non-dominant eye comprises at least one visual enhancement feature. Such visual enhancement features may be selected from the addition of at least one contour ring, the addition of jitter, the addition of counterphase motion, the addition of higher mean luminance intensity and combinations thereof. In addition, in some trials, or over the course of one or more successive or non-successive presentations, the visual stimuli provided to the non-dominant eye may have a slightly lower or a decreasing contrast and/or the visual stimuli provided to the dominant eye may have a slightly higher or increasing contrast than those provided at the start of the training session or in a preceding trial or presentation. Additional adaptations or embodiments will be readily apparent to one of skill in the art on the basis of the disclosure herein.

In alternative aspects of the present invention, the present invention relates to methods of diagnosing sensory eye dominance in a subject with or without amblyopia, by presenting a first visual stimulus to a subject's dominant eye and a second visual stimulus to the subject's non-dominant eye, wherein the first and second stimuli may be as provided above or otherwise herein. Which of the first visual stimulus and second visual stimulus that is predominantly visualized by the subject is then determined. A physical characteristic, such as contrast, of the visual stimulus that is predominantly not detected is changed or altered until the frequency of visualization of both the first and second stimuli is approximately the same. The amount of change required to achieve equal visualization is then measured. These steps are repeated for each eye and the contrast/intensity measurements compared. The eye with the higher measurement, if any, would be considered the weaker eye. Based on the discrepancy, one can diagnose the extent of SED for that location of the visual region. Such a diagnostic method may be performed in the foveal region, parafoveal region and/or the peripheral retinal region. Further, such measurements may be taken at retinal locations concentrically throughout the visual field to establish a SED profile or map for the subject with or without amblyopia, which can be used to evaluate the treatment of the subject using one or more of the methods provided herein.

In even further alternative embodiments, the present invention relates to a system for reducing sensory eye dominance or amblyopia in a subject, wherein the system provides at least a screen or visualization element adaptable to present a first visual stimulus to one eye of a subject and a second visual stimulus to a second eye of a subject; and a non-transient storage medium adaptable to present a first visual stimulus to a non-dominant eye of a subject and a second visual stimulus to a dominant eye of the subject such that visualization of the visual stimulus in the non-dominant eye of the subject is stimulated and visualization of the visual stimulus in the dominant eye is inhibited. The storage medium and visualization element may be similarly adapted or adaptable to provide one or a series of presentations or trials, in accordance with the methods herein.

Additional embodiments, adaptations and advantages of the present invention will be readily apparent to one of skill in the art, based on the disclosure provided herein.

### BRIEF DESCRIPTION OF THE FIGURES

FIG. 1 provides a conceptual cortical model of binocular interaction. (a) provides that, at the first level, monocular units with different orientation preference from each eye inhibit one another. At the second level, monocular units from each eye with the same orientation preference converge. Interocular inhibition is activated when the two eyes are stimulated with orthogonal gratings. (b) illustrates sensory eye dominance with strong inhibition of the LE. When the two eyes are presented with orthogonal gratings of equal contrast, the RE's grating (strong eye) is perceived while the LE's grating (weak eye) is suppressed due to the stronger inhibition on the LE's monocular units.

FIG. 2 provides the experimental protocols for SED and perceptual learning. (a and b) provide the orthogonal gratings used to measure the balance contrast in the RE and LE, whose difference defines the sensory eye dominance. (c) provides the push-pull protocol. The white rectangular frame acts as a cue to attract transient attention, causing the (vertical) grating in the weak eye to be perceived while the (horizontal) grating in the strong eye is suppressed. (d) provides the push-only protocol. The stimulus presentation sequence is the same as in the push-pull protocol, except no grating is presented to the strong eye.

FIG. 3 provides the outcomes of the perceptual learning protocols on SED of observers (n=7) trained with an interleaved procedure. (a) provides the average interocular balance contrast with the push-pull protocol. The interocular balance contrast obtained, respectively, with grating whose orientation was the same as, or orthogonal to, the grating used in the training, and measured before and after each day's training. The balance contrast reduces with days in training when tested with the same orientation grating. (b) provides the interocular average balance contrast with the push-only protocol. The interocular balance contrast does not change with training. (c) shows that SED (measured before each day's training session) reduces with the push-pull but not with the push-only protocol. Both (a) and (b) also include the average data of observers (n=3) trained in a sequential procedure (plus and cross symbols; error bars are not shown to reduce clutter). Error bars indicate 1 S.E.M.

FIG. 4 provides the outcomes of the perceptual learning protocols on untrained monocular and binocular functions. (a) illustrates reduction in monocular contrast threshold at the push-pull (black bars) and push-only (gray bars) training locations, in the weak and strong eye. (b) illustrates reduction in monocular orientation discrimination threshold. (c) and (d), respectively, show the reduction in stereo threshold and reaction time at the push-pull (black bar), push-only (gray bar), and an untrained (open bar) location. Asterisks (*) indicate data whose p values in a t-test are smaller than 0.05. Error bars indicate 1 S.E.M.

FIG. 5 provides additional data on balance contrast and orientation discrimination thresholds. (a and b) provides the average balance contrast at the push-pull training-location (left graph) and push-only training-location (right graph) obtained using the method of constant stimuli. (c) provides the average orientation discrimination threshold as a function of training session during the training phase for the seven observers trained with the interleaved procedure, for the push-pull (top) and push-only (bottom) protocols.

FIG. 6 provides the 2AFC stimulus presentation sequence for testing: (a) monocular contrast threshold; (b) monocular orientation discrimination.

FIG. 7 provides (a) the 2AFC stimulus presentation sequence for testing stereo threshold; and (b) the stimulus presentation sequence for testing stereo reaction times.

FIG. 8 provides the stimuli for measuring the balance contrast and the push-pull training. (a and b) provide orthogonal gratings used to measure the balance contrast in the LE and RE, respectively. (c) provides the two retinal locations, for training, where one is for the attended and the other for the unattended condition, which are simultaneously stimulated. At each location, a cue (white rectangular frame) attracts transient attention to the weak eye, causing its (vertical and near-vertical) gratings to be perceived while the (horizontal) gratings in the strong eye are suppressed. The observer discriminates the orientation of the gratings seen by the weak eye at the attended location.

FIG. 9 provides the results of the push-pull training at the attended and unattended locations. (a) provides the average interocular balance contrast at the attended location obtained, respectively, with grating whose orientation was the same as, or orthogonal to, the grating used in the training. The same interocular balance contrast is the measured contrast in the weak eye minus 1.5 log unit (fixed contrast of grating in the strong eye); whereas the orthogonal interocular balance contrast is the measured contrast in the strong eye minus 1.5 log unit (fixed contrast of grating in the weak eye). The interocular balance contrast reduces significantly with training, particularly when tested with the same orientation grating. (b) shows the average interocular balance contrast at the unattended location exhibits a similar trend as that at the attended location. (c) shows SED, defined as the difference between the same and orthogonal interocular balance contrast reduces significant at both the attended and unattended locations as the training progresses.

FIG. 10 shows the boundary contour-based SED. (a) provides stimulus with vertical grating surrounding the vertical and horizontal grating discs. The spatial phase of the vertical grating disc relative to the vertical surround is shifted to obtain the point of neutrality. (b) is similar to (a) except that the gratings are oriented 45° and 135° and the point of neutrality is obtained from the relative phase shift of the 135° grating disc. (c) shows that the BC-based SED is significantly reduced after the training at the attended location but not at the unattended location, with both stimuli (a) and (b).

FIG. 11 shows the dynamics of interocular dominance and suppression before (pre) and after (post) the training, measured with gratings whose orientations were either the same as, or orthogonal to, the training gratings. The data are plotted as a ratio of the performance of the weak eye to the strong eye. Thus, a ratio of greater than unity indicates a superior performance in the weak eye for that stimulus. (a) illustrates that the predominance ratios increase significantly with the same grating after the training at both the attended and unattended locations, indicating an improvement of the weak eye. (b) illustrates the trend of the dominance duration ratios is similar to (a). (c) shows that the dominance frequency ratios do not change significantly with training.

FIG. 12 shows the transfer of perceptual learning to stereopsis. (a) shows the random-dot stereogram used for measuring binocular disparity threshold for seeing a disc target in depth. (b) shows that binocular disparity thresholds are significantly reduced at both the attended and unattended locations after the training.

FIG. 13 shows the monocular contrast thresholds are significantly reduced after the training at the attended and unattended locations in both the weak and strong eyes. However, these generalized and small reductions are unlikely to be associated with the reduction in SED.

FIG. 14 shows the sample stimuli for measuring SED. (a) shows the LE balance contrast is obtained by varying the horizontal grating contrast while keeping the contrast of the vertical grating seen by the RE constant (1.5 log unit). The balance contrast is reached when the two eyes obtain an equal percentage of perceiving the two gratings (point of equality). (b) shows that the gratings are switched between the two eyes to obtain the RE balance contrast of the horizontal grating. The difference between the LE and RE balance contrast values defines the SED.

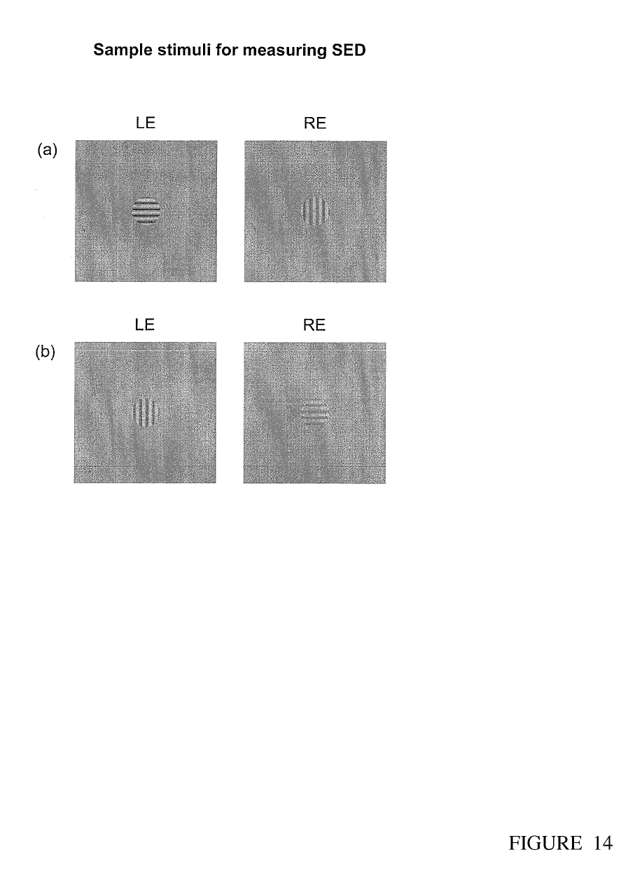

FIG. 15 provides (a) the stimulation sequence in the push-pull training protocol. A square frame is presented to the weak eye to cue attention to it, causing it to be dominant when the two eyes are subsequently stimulated with the binocular rivalry gratings. Four hundred msec later, the same sequence of event is repeated but with the grating seen by the weak eye having a slightly different orientation from the first grating seen by the same eye. The observer reports whether the first or second vertical (or near-vertical) grating has a more counterclockwise orientation, (b) the four pairs of binocular rivalry gratings used for training (and also to measure SED). These gratings are randomly intermingled in the same training block of trials, so that the weak eye is stimulated with all four grating orientations (0°, 45°, 90° and 135°).

FIG. 16 provides the orientation tuning function of early cortical neurons modeled with a Gaussian distribution function centered at the 45° orientation with a standard deviation of 37.5°. Cortical neurons with such a tuning function respond much less to the 0° and 90° grating stimuli than to the 22.5° and 67.5° grating stimuli (arrows).

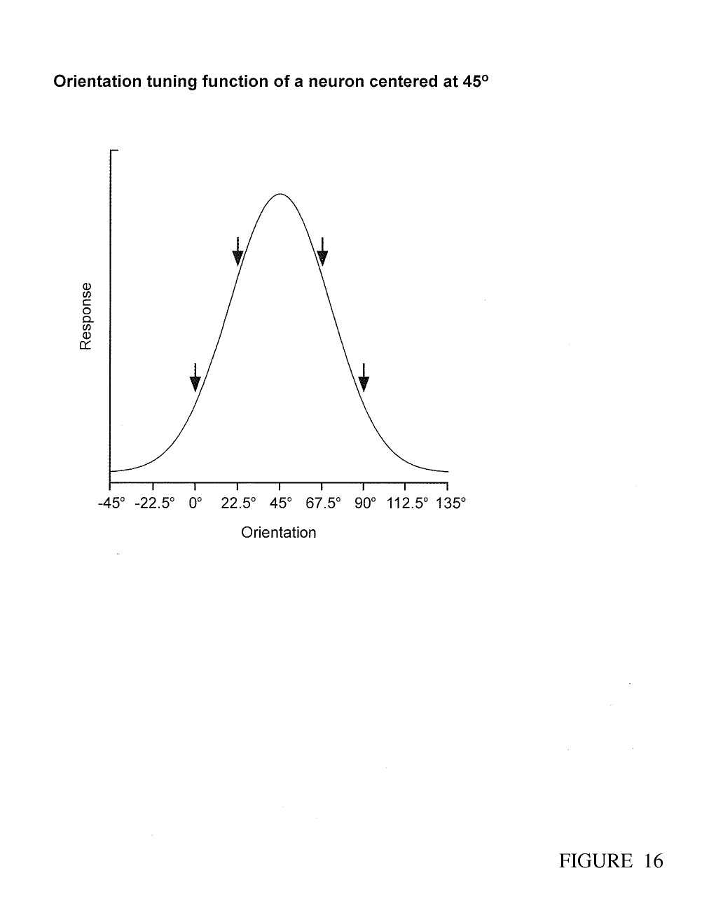

FIG. 17 (a-d) provides results showing that SED is reduced at the trained orientations (0°, 90° 45° and 135°). The notation above each graph, e.g., 0°-0°/90° in graph (a), indicates that to obtain the SED, the contrast of the 0° (horizontal) grating of the 0°/90° binocular rivalry grating stimulus was adjusted. That is, the first number is the orientation of the variable contrast grating while the second and third numbers are the orientations of the dichoptic grating test stimulus. Generally, SED reduces as the training progresses. However, within each day's training session, SED is smaller before the training than after the training.

FIG. 18 (a-b) provides results showing that SED is reduced at the untrained contrast levels (1.3 and 1.7 log units) with the 0°-0°/90° and 45°-45°/135° grating stimuli after the training phase. Training was performed with a fixed contrast level of 1.5 log unit in one half-image.

FIG. 19 provides results showing that SED is reduced at the untrained orientations (22.5° and 67.5°). The mean trained data represents the average SED from all four trained orientations (0°, 45°, 90° and 135°).

FIG. 20 provides results showing that SED is reduced at the untrained spatial frequency (6 cpd) with the 0°-0°/90° and 45°-45°/135° grating stimuli. Training was performed with a fixed spatial frequency of 3 cpd.

FIG. 21 provides results showing that training improves the weak eye's competitive advantage during extended viewing of a (0°/90° binocular rivalry stimulus. A performance ratio of larger than unity indicates an imbalance that favors the strong eye, and a performance ratio of unity indicates the two eyes are balanced. Clearly, all average performance ratios, except for the frequency performance ratio, change toward the balance level (dash line) after the training.

FIG. 22 provides (a) the random-dot stereogram used to measure stereo threshold. (b) shows that binocular disparity threshold is reduced after the training, even though the observers were never exposed to the stimulus, or task, during the training phase.

FIG. 23 shows the effect of training on the orientation discrimination thresholds during the training phase. (a) shows the change in the average orientation discrimination threshold as a function of training session for the four trained orientations (0°, 90° 45° and 135°). Each data point represents the average of the 5 blocks of training trials performed during each training session. For all orientations, thresholds decrease with training. (b) illustrates graph plots of the average orientation discrimination thresholds for one orientation, from the first and last blocks of the training session (day). There is no significant difference between the first and last blocks of orientation discrimination thresholds with the 0° and 90° stimuli. However, with the 45° and 135° orientation discrimination thresholds, performance is better in the last block of training trials than the first block.

FIG. 24 shows the correlation between binocular rivalry percepts measured over an extended viewing duration and SED. (a) illustrates the correlation between the predominance ratio (SE/WE) and SED using data provided herein. These two measurements vary in the same direction. (b) correlates the change in the predominance ratio (pre-post training) and the reduction in SED after training. A significant correlation was found, which suggests observers with more reduction in SED have a larger change in the binocular rivalry perception.

FIG. 25 illustrates the correlation between the reduction in stereo disparity thresholds and reduction in SED. It indicates observers whose binocularity became more balanced (reduced SED) also have more reduction in binocular disparity threshold (improved stereoacuity).

FIG. 26 shows the retention of learning (reduced SED). (a) illustrates the Retention Index (RI) in the fovea of eight observers tested at various days after the training phase ended. (b) illustrates the RI at the 2° parafoveal location of ten observers from data provided herein. Each observer's data are plotted with different symbols and was tested at different days after the completion of training. An RI of unity indicates the learning effect is fully maintained, while an RI of zero indicates the learning effect has dissipated.

FIG. 27 provides (a) two pairs of orthogonal gratings for measuring contrast-SED. The left pair of dichoptic stimulus measures the LE balance contrast, which is obtained by adjusting the contrast of the horizontal grating in the LE while keeping the contrast of the RE's vertical grating constant. The right pair of dichoptic stimulus is used to measure the RE balance contrast in a similar manner. The difference between the LE and RE balance contrast values defines the contrast-SED, (b) two pairs of orthogonal gratings for measuring BC-SED. During the test, one keeps the contrast levels of the vertical and horizontal gratings constant while adjusting the relative phase-shift between the horizontal grating disc and its surrounding horizontal grating. In this way, the BC strength of the horizontal grating disc is varied to obtain the balance phase-shift for the LE (left pair of stimulus) and RE (right pair of stimulus). The difference between the LE and RE balance phase-shift values defines the BC-SED, (c) the MBC rivalry stimulus, which gives rise to a stable perception of the horizontal grating disc (lasting seconds). (d) and (e) depict pairs of orthogonal grating stimuli for measuring BC-SED. They differ from those in (b) in their grating orientations. In (d), the variable phase-shifted disc is vertical and both half-images have vertical grating background. In (e), the variable phase-shifted disc is oriented 135° and both half-images have 135° oriented grating background.

FIG. 28 provides (a) sequence of stimulus presentation in the push-pull training protocol employed, using the attention cue to secure dominance of the weak eye, (b) and (c) show the stimulus presentation sequence for the MBC push-pull and BBC push-pull protocols, respectively. Instead of cueing for attention as in (a), the weak eye's half-image is presented with a strong stimulus (horizontal grating disc). The strong eye's half-image is defined by surface feature (vertical grating) in (b), and by both boundary contour and surface feature (vertical grating disc) in (c).

FIG. 29 provides the average interocular balance phase-shift data for (a) the MBC push-pull protocol and (b) BBC push-pull protocol. The interocular balance phase-shift data were obtained, respectively, with grating whose orientation was the same as, or orthogonal to, the grating used in the training, and measured before and after each day's training. Generally, the interocular balance phase-shift reduces with days in training when tested with the same orientation grating. Gray symbols in both graphs plot the average interocular balance phase-shift data (for pre-training at day 0 and post-training at day 10) that were obtained using the method of constant stimuli. (c) BC-SED reduces with days in the training for both protocols. The gray symbols reveal the data obtained with the method of constant stimuli.

FIG. 30 provides (a) and (b), respectively, plot the average data for the MBC and BBC push-pull training locations with fitted psychometric functions (cumulative normal distribution functions). Generally, the psychometric functions for the same/after data are to the left of the same/before data, indicating a reduction in the weak eye's balance contrast.

FIG. 31 provides (a) average BC-SED data tested with the stimulus with horizontal grating background using the method of constant stimuli. For training at both the MBC and BBC push-pull locations, BC-SED is significantly reduced after (post) the training. (b) average BC-SED measured using the test stimuli with vertical grating background in the pre and post training phase. BC-SED is significantly reduced at the MBC and BBC push-pull training locations. (c) average BC-SED measured with the test stimuli with oblique grating background before and after the training. A significant reduction of BC-SED is found at the BBC training location but not at the MBC training location. (d) average contrast-SED measured before and after the training phase with the test stimuli shown above. The reduction in BC-SED is significant at the BBC training location but not at the MBC training location.

FIG. 32 provides (a) the random-dot stereogram used to measure binocular disparity threshold. (b) the average stereo thresholds measured with random dot stereogram at the MBC and BBC push-pull training locations. A significant reduction in binocular disparity threshold is found at both training locations.

FIG. 33 provides (a and b) sample stimulus used for measuring SED. (a) shows that the LE balance contrast is obtained by varying the horizontal grating contrast while keeping the contrast of the vertical grating seen by the RE constant (SED stimulus-a). The LE balance contrast is reached when the two eyes obtain an equal percentage of perceiving the two gratings (point of equality). (b) The gratings are switched between the two eyes to obtain the RE balance contrast (SED stimulus-b). The difference between the LE and RE balance contrast values defines the SED. (c) Schematic of the binocular visual field indicating the 17 retinal locations tested with the stimulus sizes appropriately scaled to account for the cortical magnification factor. (d) The random-dot stereogram used to measure stereo threshold.

FIG. 34 provides binocular visual fields of individual observers showing the distributions of their (a) sensory eye dominance (SED) and (b) interocular difference in contrast threshold, at the 17 tested locations. The data are represented in gray shades that correspond to the extent and sign of SED or interocular difference in contrast threshold.

FIG. 35 provides binocular visual fields of individual observers showing the distributions of their (a) binocular disparity threshold and (b) reaction time to detect binocular depth. These data are plotted with a dark-light gray shade that represents the different gradients of the data. A smaller measured value is represented with a darker gray shade that is closer to the black background.

FIG. 36 provides the assessment of the effect of eccentricity. Data from the same retinal eccentricity for each of the measured function (SED, interocular difference in contrast threshold, binocular disparity threshold, and stereo reaction time) were averaged and plotted as a function of the tested retinal eccentricity (fovea: 0; parafovea: 2°, 4°). Only binocular disparity threshold increases significantly with retinal eccentricity. Please note that for convention, a negative value was used (of SED or interocular difference in contrast threshold) to indicate LE superiority and a positive value to indicate RE superiority.

FIG. 37 provides the correlation of the measured attributes at the 2° and 4° retinal eccentricities from either the same side of the retina, or across the fovea on the opposite side. A more gradual change (larger R² value) in SED and interocular difference in contrast threshold is found when the correlation is done on the same side of the retina (a & c), than when it is done on the opposite side (b & d).

FIG. 38 provides the correlations between the foveal and parafoveal measurements of (a) SED and (b) interocular difference in contrast threshold. The parafoveal data were derived from averaging all the 2° and 4° results (16 positions).

FIG. 39 provides the correlation between SED and interocular difference in contrast threshold in the (a) parafovea (small and large dots for 2° and 4°, respectively) and (b) fovea. Data points falling in the first and third quadrants (consistent) indicate superiority in the same eye for both measures, whereas data falling in the second and fourth quadrants (inconsistent) indicate opposite eye superiority.

FIG. 40 (a) and (b) correlate binocular disparity threshold and absolute SED, respectively, in the parafovea and fovea. (c) and (d) correlate binocular disparity threshold and absolute interocular difference in contrast threshold, respectively, in the parafovea and fovea. Generally, the correlation between binocular disparity threshold and absolute SED is higher than between binocular disparity threshold and absolute interocular difference in contrast threshold. Note that in (a) and (c), the small and large dots depict the 2° and 4° data, respectively.

FIG. 41 (a) and (b) correlate relative RT (z-score) for seeing depth and absolute SED, respectively, in the parafovea and fovea. (c) and (d) correlate relative RT (z-score) for seeing depth and absolute interocular difference in contrast threshold, respectively, in the parafovea and fovea. Generally, the correlation between relative RT (z-score) for seeing depth and absolute SED is higher than between relative RT (z-score) for seeing depth and absolute interocular difference in contrast threshold. Note that in (a) and (c), the small and large dots depict the 2° and 4° data, respectively.

FIG. 42 (a) correlates interocular difference in predominance (from tracking binocular rivalry percepts) and SED. Data falling in the first and third quadrants indicate the superior eye in SED also enjoys a higher predominance when tracking binocular rivalry. (b) comparing each observer's motor eye dominance with his/her foveal SED and mean parafoveal SED. Data falling in the inconsistent quadrants indicate the sensory and motor dominant eye is opposite.

FIG. 43 shows a random-dot stereopsis stimulus for measuring stereo threshold and the response time to seeing stereopsis. With fusion, a target (disc) in depth is seen in the middle of the target. Some amblyopic observers are not able to experience stereopsis with this stimulus, and are tested with the contour stimulus in FIG. 44.

FIG. 44 shows a contour stereopsis stimulus for measuring stereo threshold and the response time to detect depth. The two dots above and below the cross are seen in depth relative to the cross.

FIG. 45 shows a stimulus sequence for measuring SED.

FIG. 46 shows the 2AFC stimulus sequence for measuring monocular contrast threshold (in the LE). The minimum contrast required to perceive the vertical grating (in the LE) defines the monocular (LE's) contrast threshold.

FIG. 47 provides the sequence of the stimulus presentation in the push-pull protocol with BBC target. An attention cue presented to the weak (amblyopic) eye ensures that the subsequent binocular rivalry stimulus in that eye becomes dominant while the stimulus in the strong eye is suppressed. The attention cue is presented again to ensure the dominance of the weak amblyopic eye's binocular rivalry stimulus. The observer's task is to discriminate the orientation difference between the two gratings seen by the weak amblyopic eye.

FIG. 48 provides the sequence of the stimulus presentation in the push-pull protocol with MBC target, which is the same as that with the BBC stimulus in FIG. 47.

FIG. 49 shows two techniques for promoting dominance of the weak eye. The half-image viewed by the weak eye can be augmented with (a) contour ring and (b) jittering the grating within the area enclosed by the attention cue.

FIG. 50 provides the sequence of the stimulus presentation in the push-pull protocol with BBC target using a contrast discrimination task. The gratings in the weak amblyopic eye remain vertical but change in contrast in each training trial. During the training, the observer is required to discriminate between the contrast levels of the two vertical gratings in the weak amblyopic eye from the two intervals.

FIG. 51 provides the sequence of the stimulus presentation in the push-pull protocol with MBC target using a contrast discrimination task. The sequence is similar to that in FIG. 50.

FIG. 52 shows the reduction in SED as a function of the push-pull training session, respectively, for observers (a) S1, (b) S2 and (c) S3. The SED data are obtained by taking the difference in the balance contrast levels between the two eyes that were obtained after each training session.

FIG. 53 shows the stereopsis thresholds measured before and after the push-pull training phase for observers (a) S1, (b) S2 and (c) S3.

FIG. 54 shows the stereopsis response time measured before and after the push-pull training phase for observers (a) S2 and (b) S3.

FIG. 55 shows the monocular contrast thresholds measured before and after the push-pull training for observers (a) S1, (b) S2 and (c) S3.

FIG. 56 shows the average orientation discrimination thresholds obtained during the push-pull training as training progresses for observers (a) S1, (b) S2 and (c) S3.

FIG. 57 shows the average contrast discrimination thresholds obtained during the push-pull training as training progresses for observers (a) S1, (b) S2 and (c) S3.

FIG. 58 shows the balance contrast of each eye before (pre), immediately after (post), and months after (retain) the end of training. Specifically, the retention testing was performed 5 and 3 months after the end of training, respectively, for observers (a) S1 and (b) S2.

FIG. 59 shows the stereopsis threshold before (pre), immediately after (post), and months after (retain) the end of training phase. Specifically, the retention testing was performed 5 and 3 months after the end of training phase, respectively, for observers (a) S1 and (b) S2.

FIG. 60 shows the stereopsis response time before (pre), immediately after (post), and 3 months after (retain) the end of training phase for observer S2.

FIG. 61 shows the monocular contrast threshold before (pre), immediately after (post), and months after (retain) the end of training phase. Specifically, the retention testing was performed 5 and 3 months after the end of training, respectively, for observers (a) S1 and (b) S2.

### DETAILED DESCRIPTION OF THE INVENTION

The present invention relates to systems and methods for providing a push/pull perceptual learning technique to a subject demonstrating SED or amblyopia. More specifically, the weak or non-dominant eye of the subject is cued forcing it to become dominant, while visualization in the strong or dominant eye is suppressed over the course of treatment. Such systems and methods are surprisingly and unexpectedly shown herein to result in a perceptual learning and a reduction of interocular imbalance. In effect, they demonstrate a marked reduction in SED and amblyopia, as well as the improvement of visual characteristics typically associated with such disorders.

Generally, the present method relates to transiently obtaining attention to the non-dominant eye and maintaining that attention over the course of treatment. The attention can be obtained using a separate attention cue, or otherwise by the visual stimulus presented during the treatment. In either case, the visual stimuli presented during the training are oriented such that the stimulus presented to the non-dominant eye is preferred and perceived over the stimulus simultaneously presented to the dominant eye. Applicants have shown herein that this combination of the stimulation of the non-dominant eye (the push) combined with the suppression of the dominant eye (the pull) is advantageous in reducing SED. Specifically, the pull component of the method stimulates the dominant eye, while denying its retinal image from being perceived. While not intending to be bound by theory, it is believed that this reduces the transmission efficiency of the dominant eye and its effectiveness in suppressing the non-dominant eye. Applicants have shown herein, that such a technique results in reduced binocular imbalance associated with SED and/or amblyopia, and leads to improved stereopsis, improved binocular visual processing, and similar visual traits.

As noted above, prior to or in conjunction with the start of treatment, the visual attention of the observer must first be drawn to the non-dominant eye. In one optional embodiment, this is accomplished by presenting only the non-dominant eye with an attention cue. By way of example only, the attention cue may be presented as a rectangular frame of any shape, size or configuration. Specifically, though not exclusively, it may be a rectangular frame formed from solid or dashed lines. It may be provided for any amount of time that is effective to obtain the observer's attention, such as, but not limited to, less than 1 second or, alternatively, during the course of a trial. Again, the attention cue is not limited to the foregoing and may be easily adapted into any alternative configuration designed to shift the observer's focus to the non-dominant eye. In alternative embodiments, the corrective visual stimulus or presentation, itself, may act to shift the observer's attention to the non-dominant eye. Thus, in such embodiments, a separate attention cue is unnecessary.

The visual stimuli used to correct interocular imbalance are provided to either, each, or all of the foveal, parafoveal or peripheral retinal regions of the observer. With the former, for example, the observer is asked to focus in a nonius target in the foveal region. This target is then removed and replaced with one or a series of visual stimuli in the same region. For the parafoveal or peripheral retinal region, the observer focuses on a nonius fixation target in the foveal region while visual stimuli are provided at one or more eccentric retinal locations. Parafoveal or peripheral retinal visual stimuli may be provided in any area, but in certain aspects they are provided where the greatest extent of imbalance is measured. Methods for determining such areas are discussed in greater detail below.

Visual stimuli may include one isolated stimulus at the position of interest or multiple presentations simultaneously at various positions in the field of vision. Should multiple presentations be provided, it is ideal, though non-limiting, to have the observer focus on a single area with the greatest SED (the attended area) and ignore presentations in the other regions of the field of vision (the nonattended area). Applicants have shown herein that, while focusing on one area (the attended area) reduces SED in that area, the use of multiple stimuli in unfocused or non-attended areas also improves SED.

The visual stimuli, which may be non-identical patterns, in certain embodiments may include one or multiple orthogonal grating disc pairs with one disc in each pair being provided to one eye and the second disc being provided to the second eye. The pairs are specifically oriented to maintain the focus of the observer with his or her non-dominant eye. In certain non-limiting embodiments, the discs are approximately 1.25°, 3 cpd, 35 cd/m² in size with a series of parallel lines extending from one end of the disc to the other. The pair of discs are orthogonal in the sense that the grating is oriented in one direction in one eye and is oriented in a substantially perpendicular direction in the other eye. The discs may be presented to the observer for any amount of time to maintain the observer's focus on the non-dominant eye while suppressing the dominant eye and/or to accomplish one or more of the effects and/or advantages provided herein. In one non-limiting embodiment, the visual stimuli are presented to the observer for less than 1 second or about 500 ms before being removed.

Visual stimuli provided in the foveal or any other extrafoveal retinal region, for example, may be presented to the non-dominant eye at any visual angle. In certain non-limiting embodiments, for example, the discs are provided as 2-D or 3-D orthogonal pairs having the grating oriented at 0°, 45°, 90°, 135°, or 180° angles. In embodiments of the present invention where visual stimuli are presented in succession, the stimuli may have the same or a different angle with each presentation. That is, a first stimuli may be presented at a first orientation angle and a second stimuli may be presented at a second orientation angle that has angular variation from the first stimuli of between greater than 0 about degrees to less than about 180 degrees. Such a change in angle may be detectable, and in certain embodiments reportable, by the observer.

In successive presentations of the visual stimuli, over a single session or trial or over two or more trials, one or more additional physical features of the visual stimuli, in addition to or other than orientation angle, may be changed, added, or deleted. In certain embodiments, such features may include, but are not limited to, changes in contrast of the visual stimuli and/or mean luminance intensity, which in certain embodiments may or may not be detectable by the subject. In other embodiments, the change may be the addition of one or more signal enhancers, such as but not limited to, the addition of a contour ring to the visual stimuli, the addition of visual stimuli jitter, the addition of visual stimuli counterphase motion, or the like. As used herein, the term “jitter” refers to the small magnitudes of displacement of the visual stimuli in organized or random directions. The level of displacement, direction of movement and/or frequency of motion may be any level such that jitter may be observed by a subject. In certain non-limiting embodiments, the displacement may be at, about, or within 0.1°. They may occur at a speed of at, about or within 4°/sec and/or at and a frequency of at, about, or within 5 Hz. As used herein the term, “counterphase motion” refers to the movements of the grating inside each disc stimulus in a back-and-forth direction. The level of such movements, e.g. speed and frequency, may be any amount such that the counterphase motion may be observed by a subject. In certain non-limiting embodiments, the counterphase motion may occur at a speed of at, about, or within 4°/sec and/or at, about or within a frequency of 5 Hz. Such augmentations may be detectable, and in certain embodiments reportable, by the observer.

Applicants to the present invention have found that decreasing the contrast of the visual stimuli in the non-dominant eye and/or increasing the contrast of the visual stimuli in the dominant eye, while maintaining visualization of the non-dominant eye's stimuli is possible as the training sessions progress. Doing so has the effect of making the push-pull training task more difficult, which may improve one or more of the desired corrective effects discussed herein. Applicants have further found that enhancing visual signals in the non-dominant eye, such as the addition of a contour ring, jitter, counterphase motion, higher mean luminance intensity or the like, also contributes to SED and amblyopia reduction when used, alone or, in particular, when used in conjunction with contrast reduction.

Visual stimuli, particularly grating discs, may be provided against a solid background or against a grating background having the same or different orientation to the grating disc. With the former, the blank background may be gray or subdued background or any other color (such as a light color) where the visual stimulus may be detectable. With regard to the latter, a grating background having substantially the same orientation is presented to each eye of the observer. The grating disc provided in visual presentation to the non-dominant eye is preferably, though not exclusively, in an oblique or orthogonal orientation to the background grating. Such an orientation is shown herein to act as an attention cue and to maintain the visualization through the non-dominant eye throughout the course of treatment. Accordingly, without limiting the invention, in one aspect such an orientation may be simultaneously used as the attention cue for shifting an observer's focus to the non-dominant eye. The visual stimulus to the dominant eye may be solely a background grating with an orientation similar to the presentation on non-dominant eye. Alternatively, a grating disc may be disposed on the background grating in substantially the same grating orientation as the background. The disc may be outlined or otherwise indicated by a phase shift from the background grating in any amount between about 0° to 180°.

As used herein, the terms “trial” or “training session” may refer to a singular or multiple presentations of one or more of the visual stimuli in accordance with the teachings herein. In certain non-limiting embodiments, a trial or single training session may refer to a time period marked by the presentation of an initial attention cue to establish visualization in the non-dominant eye of the subject, presentation of one or more of a series of visual stimuli (with optional and intermittent attention cue presentations to maintain visualization in the non-dominant eye), followed by trial termination. Non-limiting examples of such trials or training sessions are provided below and in the Examples herein. In certain embodiments, the present invention contemplates one or more trials or training sessions within a given time period. Such trials may be conducted successively (i.e. one immediately or nearly immediately after the other), after a brief rest period within the same day, after a long rest period within the same day, or over the course of several days. To this end, and in certain non-limiting embodiments, trials may be grouped such that a certain number are performed on or over the course of one day (or a first period of time) and a certain number are performed on or over the course of a second day (or a second period of time), etc. The number of trials, groups of trials, length of trials, length of treatment, or the like, may be determined based on one or more factors, such as, but not limited to, the diagnosis of SED in the patient, aggressiveness in treatment methods, the desired level of correction, and/or the like. To this end, the present invention is not limited to any particular trial, or treatment regiment.

Based on the foregoing, in one embodiment of the present invention, an observer that has been diagnosed with SED or amblyopia in accordance with one or more of the procedures provided herein (see below) aligns his or her eyes with a nonius fixation target in the foveal region. A first attention cue is presented to the non-dominant eye then optionally removed. This is followed by a pair of dichoptic orthogonal gratings, where the non-dominant eye's grating is substantially vertical or at an oblique angle and the dominant eyes grating is substantially horizontal or at an orthogonal oblique angle to the grating in the non-dominant eye. The pair of gratings is then removed. The attention cue is then optionally presented and removed to the non-dominant eye and is followed by a second set of dichoptic gratings, which may be presented with a slightly different angular orientation or contrast. The second set of gratings is then removed, and the trial is terminated. The observer may optionally report whether the first or second grating seen with the non-dominant eye had the slight orientation change, or contrast change, or other change or addition provided herein either during or after the trial. In some trials, the visual stimuli provided to the non-dominant eye may be provided with signal enhancement (e.g. contour ring, jitter, counterphase motion, higher mean luminance intensity, etc), as compared to a previous trial. In yet other trials, the visual stimuli provided to the non-dominant eye may have a slightly lower contrast and the visual stimuli provided to the dominant eye may have a slightly higher contrast than those provided at the start of the training session or a previous trial or training session.

In an alternative embodiment of the present invention, the observer aligns his or her eyes with a nonius fixation target in the foveal region. When the trial is initiated, a pair of attention cues are presented then removed from the weak eye in the attended and unattended positions. This is follow by a pair of dichoptic orthogonal gratings. The weak eye's gratings are similarly oriented and are either both vertical or both oblique, while gratings presenting to the strong eye are either both horizontal or both at orthogonal oblique angles to the grating in the non-dominant eye. Each grating is simultaneously presented for a length of time within the range provided herein. After removal of dichoptic orthoganol gratings, the same attention cues are presented and removed again, followed by a second pair of dichoptic gratings with the slight different orientation or contrast in the weak eye. The trial is then terminated, and the observer optionally reports whether the first or second grating had the slight orientation change, or contrast change, or other change or addition provided herein either during or after the trial. Again, in some trials, the visual stimuli provided to the weak eye may be provided with signal enhancement (e.g. contour ring, jitter, counterphase motion, higher mean luminance intensity, etc.), as compared to previous trials. In yet other trials, the visual stimuli provided to the weak eye may have a slightly lower contrast and the visual stimuli provided to the strong eye may have a slightly higher contrast than those provided at the start of the training session, or previously trials or training sessions.

In another non-limiting embodiment of the invention, the observer aligns his or her eyes with a nonius fixation target in the foveal region. When the test is initiated, the nonius fixation target is removed and is replaced by the attention cue. After the cue is removed, a pair of dichoptic orthogonal gratings at any angle are presented and removed. The attention cue is presented again and removed, followed by the presentation of a second pair of dichoptic gratings having a slightly different orientation or contrast from the grating shown in the first presentation. The trial is then terminated, and the observer optionally reports whether the first or second grating had the slight orientation change, or contrast change, or other change or addition provided herein either during or after the trial. In an alternative design, the attention cue at each presentation is retained and seen together with the stimulus provided to the non-dominant eye. Again, in some trials, the visual stimuli provided to the non-dominant eye may be provided with signal enhancement (e.g. contour ring, jitter, counterphase motion, higher mean luminance intensity, etc.), as compared to a previous trial. In other trials, the visual stimuli provided to the non-dominant eye may have a slightly lower contrast and the visual stimuli provided to the dominant eye may have a slightly higher contrast than those provided at the start of the training session, or provided in a previous trial or training session.

In a further embodiment of the present invention, the visual stimulus has a horizontal or oblique grating disc surrounded by a vertical or orthogonally oblique grating background as a visual stimulus for the non-dominant eye and a homogeneous vertical or orthogonally oblique grating for the dominant eye. The trial begins with fixation at the nonius target. Then, at the training location, stimulus is presented and removed. A second stimulus is presented and removed where the grating of the disc in the second presentation has a slightly different orientation and/or contrast from the first. The trial is then terminated, and the observer optionally reports whether the first or second grating had the slight orientation change or other change or addition provided herein either during or after the trial. Again, in some trials, the visual stimuli provided to the non-dominant eye may be provided with signal enhancement (e.g. contour ring, jitter, counterphase motion, higher mean luminance intensity, attention cue, etc.), a compared to a previous trial. In yet other trials, the visual stimuli provided to the non-dominant eye may have a slightly lower contrast and the background gratings provided to both eyes may have slightly higher contrast than those provided at the start of the training session, or provided in a previous trial or training session.

In a further embodiment of the present invention, the visual stimulus had a horizontal or oblique grating disc surrounded by a vertical or orthogonally oblique grating background as a visual stimulus for the non-dominant eye. The visual stimulus for the dominant eye include a background and disc having the same orientation as each other as the background for the non-dominant eye. The grating disc was created by phase-shifting a circular region of the vertical grating surrounding it by a phase-shift between 0°-180°. The trial begins with fixation at the nonius target. Then, at the training location, stimulus is presented and removed. A second stimulus is then presented and removed where the grating of the disc in the second presentation has a slightly different orientation and/or contrast from the first. The trial is then terminated, and the observer optionally reports whether the first or second grating had the slight orientation change or other change or addition provided herein either during or after the trial. Again, in some trials, the visual stimuli provided to the non-dominant eye may be provided with signal enhancement (e.g. contour ring, jitter, counterphase motion, higher mean luminance intensity, attention cue, etc.), as compared to a previous trial. In yet other trials, the visual stimuli provided to the non-dominant eye may have a slightly lower contrast and the background gratings provided to both eyes may have slightly higher contrast than those provided at the start of the training session, or provided in previous trials or training sessions.

Any one of these trials, or adaptations thereof based on the disclosure provided herein, may be performed between 100-1,000 trials per session; 250-1,000 trials per session; 500-1,000 trials per session, or 600-1,000 trials per session per day over 7-15 days. The duration of the treatment and number of trials are not limited to such amounts and will depend on the magnitude of the deficit (SED). To this end, the number of trials performed and length of the treatment may be of any amount to achieve the desired reduction of SED or otherwise to improve the visual characteristics associated with SED and/or amblyopia.

The trials may be conducted on any system, particularly a computerized system, having hardware and software capabilities to provide such visual stimuli in accordance with the teachings herein. To this end, the present invention may include a computer program product and a non-transient storage medium or process with a computer program stored thereon. The program is adapted, when loaded and executed on a computer, to perform the inventive method for reducing binocular imbalance and/or sensory eye dominance or the associated visual characteristics provided herein. While not limited thereto, the program may be performed on any device having computer-based hardware capable of generating stereoscopic 3D displays or a 2D display that gives the appearance of a 3D image. Such devices include, but are not limited to, any device having one or more display screens (e.g. CRT, LCD, etc.) adapted to present each of the attention cues and/or visual stimuli in accordance with the teachings herein. In certain aspects, the device may be adapted for segregated viewing by each eye, i.e. the non-dominant eye views one screen, portion of a screen, or visual stimuli while the dominant eye views another. One example of such a device or a component of a device includes a halposcope or haploscopic minor system, where images presented on one or more screens are displayed only to the targeted eye using a mirror or system of minors. The device may, alternatively, include a pair of glasses equipped with one or more screens adapted provide different images to each eye in accordance with the teachings here. Additional devices will be readily apparent to one of skill in the art, based on the disclosure provided herein.

The device may also contain one or more features for observer or a subject's feedback. By way of non-limiting example, in certain aspects, the system or device may include a button or some other actuatable mechanism where, when subject feedback is requested during a trial, actuation of the mechanism serves to provide such feedback.

Prior to, during, and/or after the treatment, the extent of a subject's SED may be quantified using any standard technique for measuring binocular imbalance. Such measurements may be used to establish a baseline binocular imbalance and to track the progress of the subject through the treatment regimen. To this end, a preliminary measurement may be taken prior to treatment and compared against subsequent measurements taken before and/or after each course of treatment or periodically during the treatment process. Such information can also be used to determine whether additional or further courses of treatment are desirable or if the subject experiences relapse after the treatment is complete.

While any method of measuring such imbalance may be used, in certain non-limiting aspects SED is measured using a binocular rivalry stimulus with varying intensities or contrasts between half images. Generally, different stimuli are presented to both the dominant and non-dominant eye where only one of the stimuli is detected. Sometimes, a mixture of both eyes' stimuli is detected. In this event, the observer chooses the predominant orientation seen. The contrast and/or intensities of the non-detected stimuli is then altered in a gradually increasing manner until each eye's stimulus has an equal chance to be seen. This test is performed for both eyes to establish a collective right eye and left eye balance contrasts.

The balance contrast may be measured in the foveal region of the eye or at varying degrees from the foveal region in the parafoveal and/or peripheral retinal regions. The stimuli are preferably, though not exclusively, provided as gratings against either a blank or grating background. Depending upon the type of background, the gratings may be parallel or at an angle to each other. By way of example, in certain embodiments the stimuli of each eye are orthogonally oriented such that the grating in one eye is approximately 90° to the other.

In one non-limiting embodiment, the balance contrast is measured in each eye. Specifically, the subject is presented with dichoptic orthogonal gratings against a blank backdrop (as defined above), where the first eye is focused on, typically vertical, grating with a constant contrast. The contrast of the second grating is increased until the observer reports an equal chance of visualizing the constant contrast grating with the first eye and the variable contrast grating with the second eye. This establishes the balance contrast of the second eye. The technique then reversed to establish the balance contrast of the first eye, i.e. the second eye visualizes the constant contrast grating and the first eye the variable one. The eye with the higher balance contrast is considered the non-dominant eye.

In certain embodiments of the foregoing, the constant contrast grating is provided as a vertical grating disc and the variable contrast grating as a horizontal grating disc. The present invention is not limited to vertical and horizontal gratings and may be adapted to provided pairs of gratings with any angular orientation, where the two gratings are or may not be orthogonally oriented.

An alternative measurement method similarly detects interocular imbalance. More specifically, a grating background is presented to each eye of the observer with a pair of dichoptic orthogonal grating discs within each field. The background is preferably provided to both eyes in the same orientation, which may be vertical, horizontal or oblique. The disc in a first eye is orthogonal to the background grating. The disc in the second eye is parallel to the background grating with a variable phase-shift (0-180 degrees) relative to the background. The phase shift of this latter grating is increasingly adjusted until the observer acknowledges an equal chance of seeing both discs. This establishes the balance phase shift of the second eye. The measurement is then reversed to determine the balance phase shift of the first eye, and the eye with the higher balance phase shift is considered the non-dominant eye.

SED balance or phase-shift measurements may be taken in the foveal region (0°) or at varying concentric locations therefrom in the parafoveal and peripheral retinal regions. In one aspect, the SED region is taken at a 2° eccentric retinal location at one position or concentrically throughout the visual field, i.e. 0°, 45°, 90°, 135°, 180°, 225°, 270°, 315° around the foveal region. In further embodiments, a localized SED map can be obtained for each observer by taking SED measurements at increasing eccentric retinal locations through the field of vision. By way of non-limiting example, such measurements may include concentrical measurements through the field vision at 1°, 2°, 4°, 8° 10°, etc. from the foveal region. This map may be used as a basis for selecting locations that may be targeted for treatment, or otherwise to measure the effects of the treatment at one location (an unattended location) when treatment is provided to another (the attended location).

The following are examples of the invention and are not to be construed as limiting.

### EXAMPLES

### Example 1

### Push/Pull Protocol Using an Attention Cue Preceding Competitive Stimulation

A. Experimental Procedures

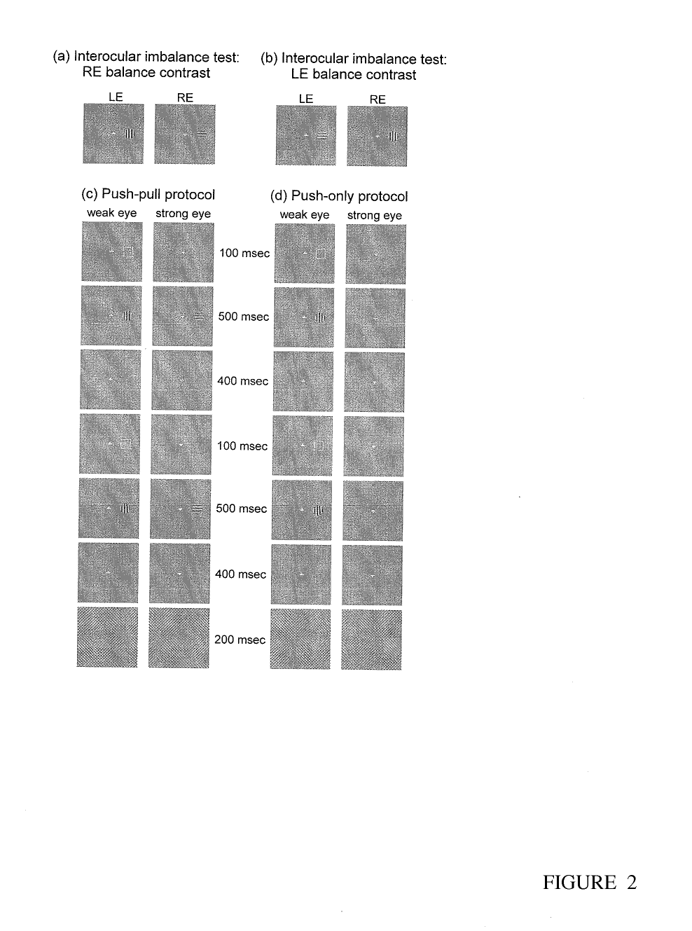

A Macintosh computer running Matlab and Psychophysics Toolbox generated the stimuli on a flat CRT monitor. All observers (one author and nine naïve observers with informed consent) had clinically normal binocular vision. The experiments performed conformed with the regulatory standards of the Institutional Review Board of the University of Louisville and of Salus University. SED was first measured with vertical and horizontal grating discs at eight concentric retinal locations 2° from the fovea (FIGS. 2a and 2b). Two locations with the largest SED were chosen for the training.

All ten adult observers had normal or corrected-to-normal visual acuity (at least 20/20), normal color vision, clinically acceptable fixation disparity (<8.6 arc min), stereopsis (<20 arc sec), and passed the Keystone vision screening tests. During the experiments they viewed the monitor through a haploscopic minor system attached to a head-and-chin rest from a distance of 85 cm.

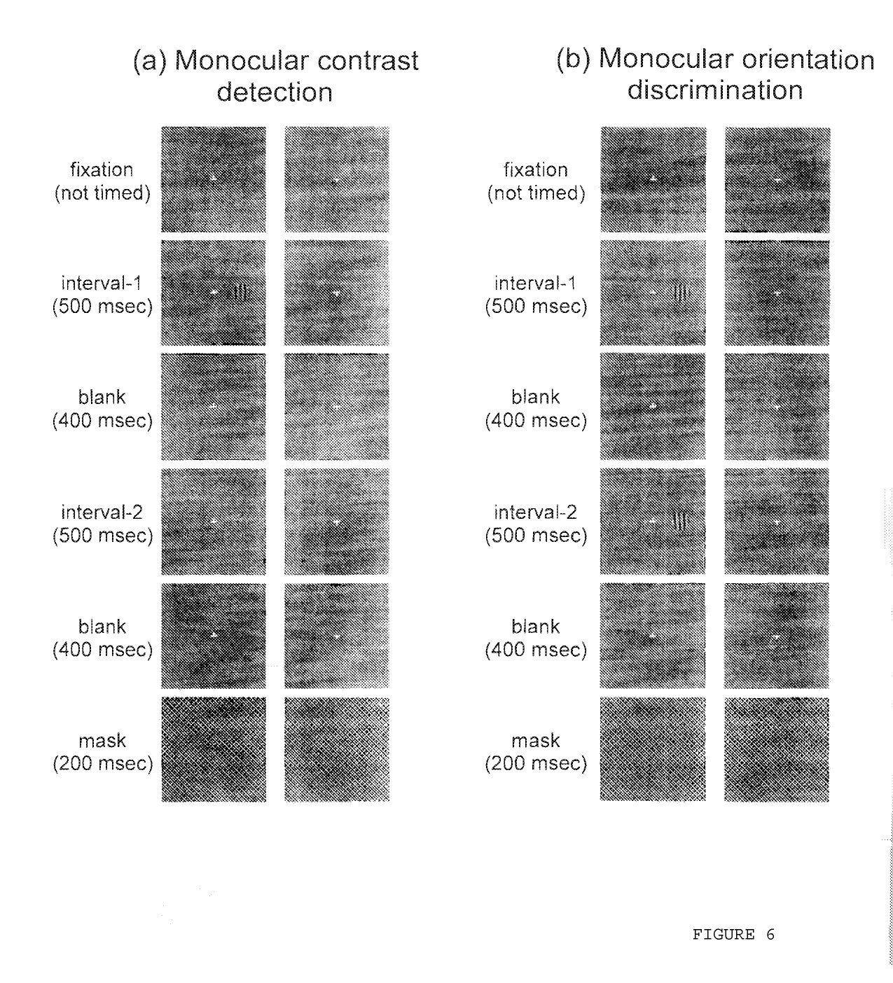

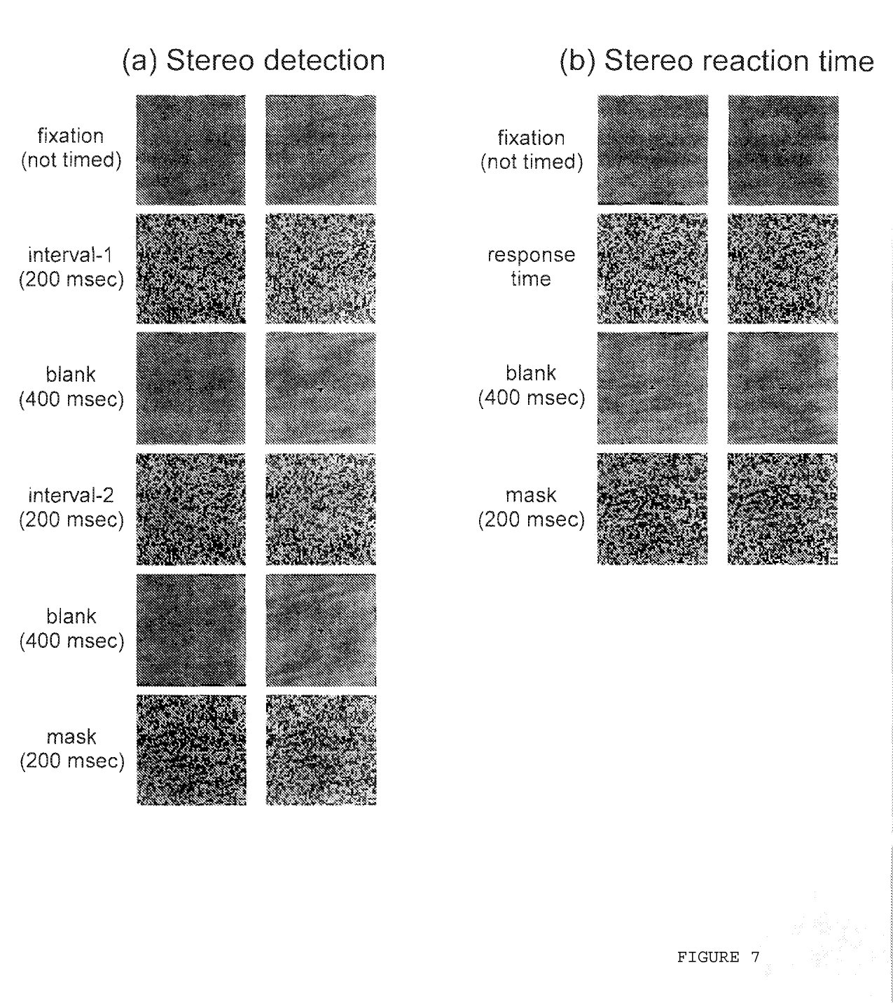

Seven naïve observers were trained in an interleaved procedure wherein both push-pull (FIG. 2c) and push-only (FIG. 2d) protocols were implemented on the same day. During the training, and for each observer, the push-pull protocol was assigned to one retinal location while the push-only protocol to the second retinal location. To accomplish this, each observer came to the laboratory for a one-hour morning session and a one-hour afternoon session (12 blocks/session) for a total of 10 days. The sequence of selecting the training protocol (push-pull versus push-only) for each session was counterbalanced with an ABBA within-subject design. To monitor the learning progress, observer's balance contrast was measured before each morning's training session, and after each afternoon's training session. To further assess the learning effect, three sets of tests were run in the pre- and post-training phase: (a) SED with 45° and 135° grating discs; (b) monocular contrast thresholds and orientation discrimination thresholds with vertical and horizontal grating discs (see FIG. 6); (c) stereo threshold and reaction time (FIG. 7).

For the interocular imbalance test, the stimulus comprised a pair of dichoptic vertical and horizontal sinusoidal grating discs (3 cpd, 1.25°, 35 cd/m²). The contrast of one grating was fixed (1.5 log unit) while the other varied (0-1.99 log unit). A trial began with central fixation on the nonius target (0.45°×0.45°, line width=0.1°, 70 cd/m²) and the presentation of the dichoptic orthogonal gratings (500 msec), followed by a 200 msec mask (7.5°×7.5° checkerboard sinusoidal grating, 3 cpd, 35 cd/m², 1.5 log unit). The observer responded to his/her percept by key presses. The horizontal grating contrast was adjusted after each trial until equal predominance was achieved using the QUEST procedure (50 trials/block). When the horizontal grating was presented to the LE its contrast at equal predominance is referred to as the LE's balance contrast. Then the gratings were switched between the eyes to obtain the RE's balance contrast. Their difference is defined as SED.

In the pre-training phase, SED was measured at eight concentric retinal locations (0°, 45°, 90°, 135°, 180°, 225°, 270°, and 315°) 2° from the fovea. Two locations with the largest SED were chosen for the training. SED at the two training-locations were further tested with: (i) 45° and 135° orthogonal gratings; (ii) the method of constant stimuli instead of the QUEST procedure. One grating (e.g., vertical) contrast was fixed at 1.5 log unit, while the other (horizontal) adopted one of seven levels (1.2-1.8 log unit). Each trial was repeated 7 times/block over 6 blocks. These two measures were performed again in the post-training phase. Separately, SED was measured at four locations (±45° adjacent to the trained-locations after the training. During the training-phase, the SED at the two training-locations were measured with horizontal/vertical gratings before and after each day's training session using the QUEST procedure.

To test monocular contrast threshold and orientation discrimination at the 2 training locations, the monocular sinusoidal grating (35 cd/m², 3 cpd, 1.25°, 500 msec) was either horizontal or vertical for the contrast sensitivity test, and near-vertical or near-horizontal for the orientation discrimination test (contrast=1.5 log unit). The fellow eye viewed a homogeneous gray (blank) field. Each test was conducted using the 2AFC method in combination with the QUEST procedure (FIG. 6). Each eye/location/orientation was tested separately in different blocks (50 trials/block), both in the pre-training and post-training phases.

For the contrast threshold test, the temporal sequence of the 2AFC stimulus presentation was: fixation, interval-1 (500 msec), blank (400 msec), interval-2 (500 msec), blank (400 msec), and mask (7.5°×7.5° checkerboard sinusoidal grating, 3 cpd, 35 cd/m², 1.5 log unit, 200 msec). The grating was presented at only one interval while the other interval had a blank field. The observer responded to seeing the grating either in interval-1 or -2 by key press, and an audio feedback was given. Grating contrast was adjusted after each trial (by QUEST) to obtain threshold.

For the orientation discrimination test, the temporal sequence of the 2AFC stimulus presentation was the same as in the contrast threshold test. This time however, one interval had a grating whose orientation was slightly different from that in the other interval. The observer responded to seeing the grating whose orientation was more counterclockwise by key press, and an audio feedback was given. Grating orientation was adjusted after each trial (by QUEST) to obtain threshold.

For the stereo tests, an untrained location with the least SED was also measured. All seven observers participated in the first two sets of tests and five in the third set. Additionally, SED with horizontal and vertical gratings were measured before and after the training at locations (±45° adjacent to the two training locations and tested on all seven observers.

Separately, three observers were trained with the push-pull protocol for 10 days, followed by the push-only protocol for a subsequent 10 days (sequential procedure). They received one hour of training during each daily session, and were assessed for the learning effect on SED. Data from both groups of observers were pooled separately for statistical analysis.

B. The Push-Pull Training Protocol

The trial began with fixation at the nonius target and the presentation of an attention cue (1.25°×1.25° frame with dash outline, width=0.1°, 1.56 log unit, 70 cd/m²) for 100 msec. After a 100 msec cue-lead-time, the first dichoptic gratings (500 msec, 1.25°, 3 cpd, 35 cd/m²) were presented. The same 100 msec cue was presented again 400 msec later, followed by a 100 msec cue-lead-time, and the second dichoptic gratings with a slightly different orientation in the weak eye (500 msec). Four hundred msec later, a 200 msec checkerboard sinusoidal grating mask (7.5°×7.5°, 3 cpd, 35 cd/m², 1.5 log unit) terminated the trial. The contrast values of the dichoptic gratings were those that led to equal predominance with the interocular imbalance test. The observer reported whether the first or second grating had the slight counterclockwise orientation, and an audio feedback was given. (Before the proper training, it was determined for each observer whether the cue successfully suppressed the grating viewed by the strong eye.). The orientation discrimination threshold was obtained using the QUEST procedure. Twelve blocks (50 trials/block) were performed in each training session.

C. The Push-Only Training Protocol

The procedure was identical to the push-pull protocol with an important exception. Instead of presenting a pair of dichoptic gratings to the training-location, only a monocular grating was presented to the weak eye's training-location while the corresponding location in the strong eye viewed a homogeneous gray (blank) field.

D. Results

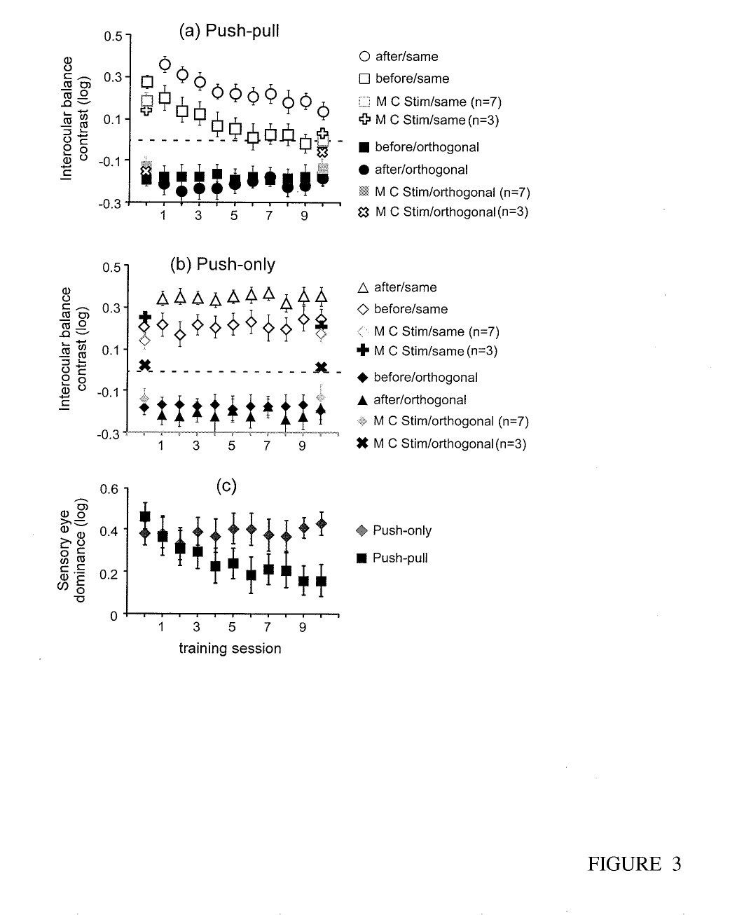

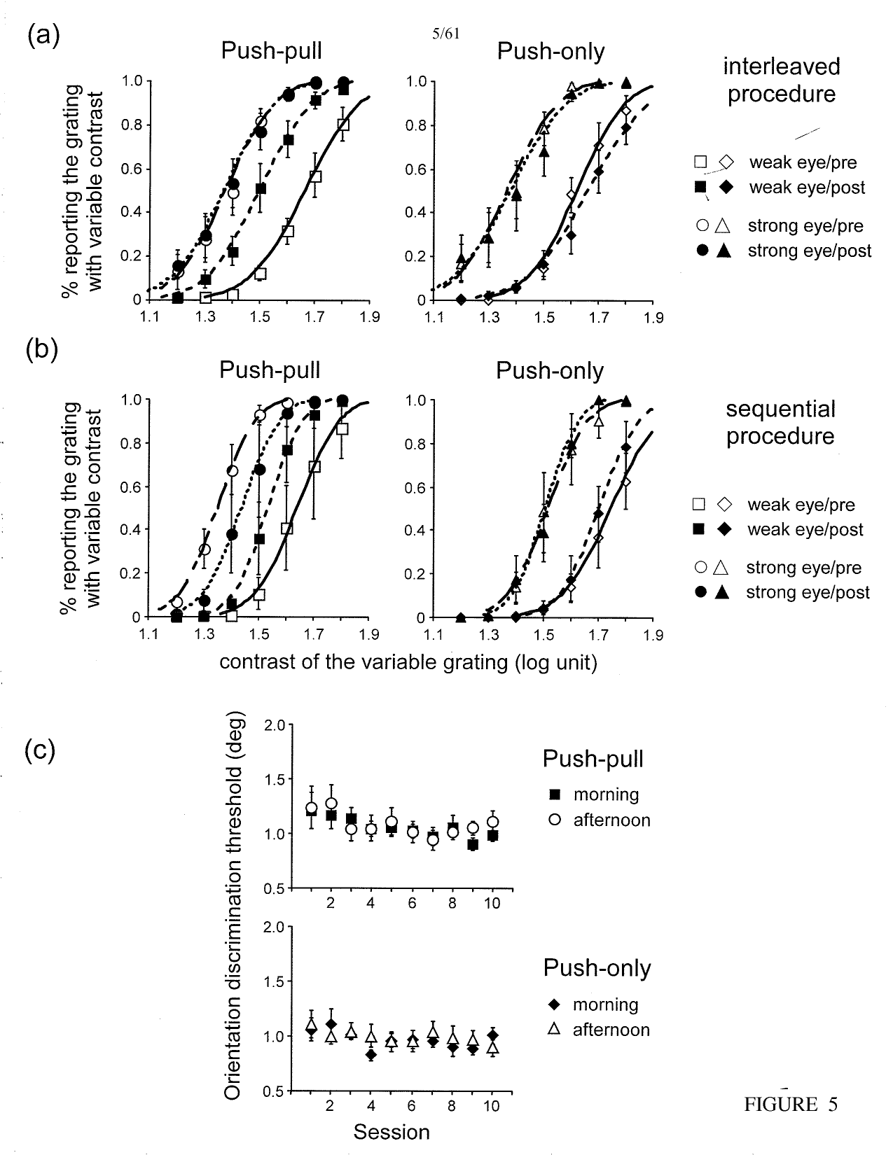

As illustrated in FIG. 3a, with the push-pull protocol the same interocular balance contrast (open symbols) declines as training progresses [before: slope=−0.026, R²=0.881, p<0.001; after: slope=−0.021, R²=0.895, p<0.001], indicating perceptual learning. However, the orthogonal interocular balance contrast (filled symbols) changes little [before: slope=−8.82×10⁻⁵, R²=0.001, p=0.919; after: slope=0.004, R²=0.297, p=0.103], suggesting the learning effect is limited to the trained stimulus orientation and eye. Also measured was the balance contrast using the method of constant stimuli before and after the entire training period. From the psychometric functions obtained (FIG. 5a) the interocular balance contrast (gray symbols, FIG. 3a) was calculated, which confirms a significant learning effect for the same [t(6)=4.318, p=0.005] but not for the orthogonal interocular balance contrast [t(6)=0.218, p=0.835].

The push-only protocol (FIG. 3b), however, shows no learning [same interocular balance contrast: before: slope=0.003, R²=0.279, p=0.095; after: slope=0.001, R²=0.028, p=0.646; orthogonal interocular balance contrast: before: slope=−0.001, R²=0.079, p=0.403; after: slope=0.001, R²=0.038, p=0.587]. The interocular balance contrast obtained by the method of the constant stimuli also fails to demonstrate a significant learning effect (t-test, p>0.05).

SED was then calculated, i.e., the difference between the same and orthogonal interocular balance contrast values in FIGS. 3a and 3b. FIG. 3c plots the SED obtained before each day's training session. The push-pull protocol significantly reduces SED (black squares, slope=−0.026, R²=0.850, p<0.001), but not the push-only protocol (gray diamonds, slope=0.004, R²=0.293, p=0.086). Similar results were obtained (not shown) from the SED measured after each day's training session (push-pull: slope=−0.025, R²=0.896, p<0.001; push-only: slope=−0.001, R²=0.012, p=0.761). Essentially, the experiment reveals that repeatedly suppressing the image in the strong eye from perception, i.e., “negatively” stimulating the strong eye, is necessary to significantly reduce SED.

FIGS. 3a and 3b reveal the magnitudes of the interocular balance contrast are larger after, than before, each daily training session with both push-pull [same: F(1,6)=92.435, p<0.001; orthogonal: F(1,6)=3.617, p=0.106, 2-way ANOVA with repeated measures] and push-only [same: F(1,6)=46.802, p<0.001; orthogonal: F(1,6)=4.464, p=0.079] protocols. The after/before differences do not vary significantly with the number of training sessions [interaction effect between the after/before and session, p>0.05]. The after/before difference in magnitude is significantly larger with the same, than with orthogonal stimuli, in the push-pull [F(1,6)=56.935, p<0.001, 2-way ANOVA with repeated measures], and push-only [F(1,6)=27.576, p=0.002] training. This is highly suggestive of stimulus orientation and eye specificity. However, this after/before difference is unlikely to be caused by fatigue during the afternoon session, as the orientation discrimination threshold data are similar between the morning and afternoon sessions (FIG. 5c). Rather, the after/before difference in interocular balance contrast resembles the performance deterioration observed during training of texture discrimination.

Separately, three other observers were trained on 10 days of push-pull protocol, followed by 10 days of push-only protocol (sequential procedure). FIGS. 3a and 3b plot the average interocular balance contrast data obtained with the method of constant stimuli (plus and cross symbols; also FIG. 5b). They show a similar trend as the seven observers' data [Push-pull: same, t(2)=4.052, p=0.056, orthogonal, t(2)=−3.497, p=0.073; Push-only: same, t(2)=0.895, p=0.465, orthogonal, t(2)=0.325, p=0.776].

Besides the balance contrast measurements, three sets of pre- and post-training tests were conducted on the observers with the interleaved training procedure. The first set of tests evaluated the hypothesis that the underlying plasticity occurs mainly in the early visual cortex, by focusing on the location and orientation specificity of learning. A finding that no learning occurs at the push-only training location could also indicate that learning at the push-pull location cannot be transferred to another training location. This suggests learning at the push-pull location occurs at cortical areas where the local feature information has not been integrated across a larger visual field. To support this, it was examined whether the learning is transferable to an untrained retinal location 1.53° from the trained location with the same eccentricity. SED reduction (0.011±0.033 log-unit) was found to be much smaller than at the trained location (0.304±0.043 log-unit) [t(6)=6.418, p=0.001]. The orientation specificity of learning was further investigated by narrowing the test orientation from 90° to 45° by measuring SED at the trained location using 45°/135° dichoptic gratings. Only a small reduction in SED was found (0.021±0.048 log-unit).

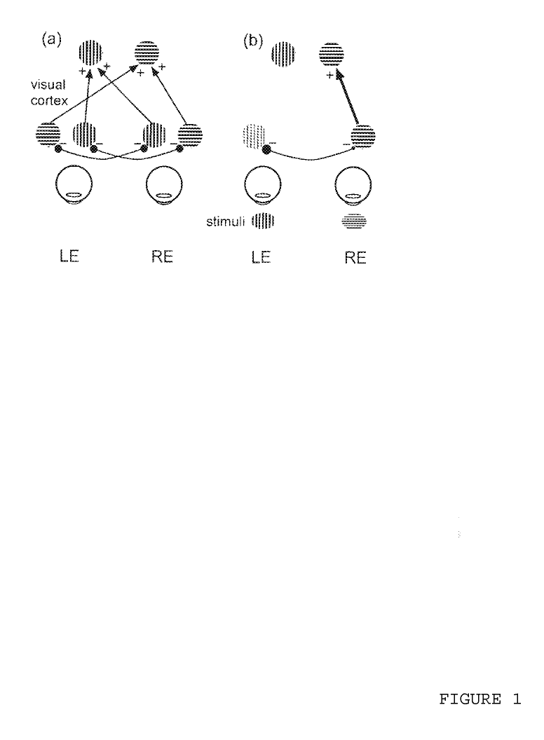

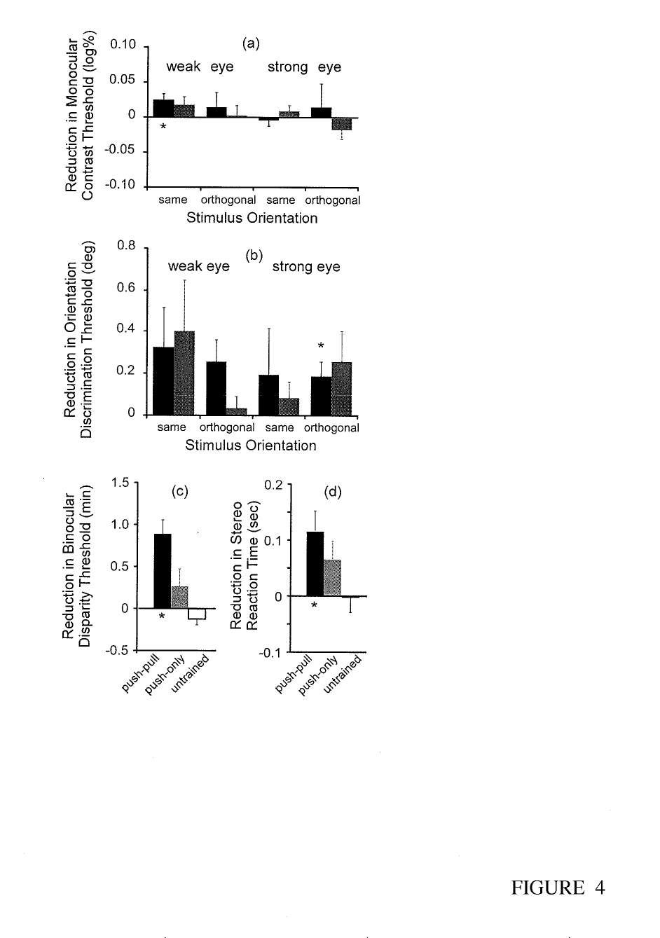

The second set of tests investigated whether the learning is accompanied by: (i) reduced efficiency of the strong eye, and/or (ii) increased efficiency of the weak eye (FIG. 1a). Such modifications in monocular efficiency can be reflected in corresponding changes in monocular contrast and orientation discrimination thresholds after training. Monocular contrast thresholds at the push-pull and push-only locations were measured using either grating with the same orientation as, or orthogonal to, the orientation of the weak eye's training grating. FIG. 4a shows threshold reduction in all, except in the strong eye (same/push-pull and orthogonal/push-only); however, the reduction is much smaller than the reduction in SED at the push-pull location (FIG. 3a). This suggests modifications of efficiency within each ocular pathway are unlikely to be the main factor responsible for learning. Similarly, monocular orientation discrimination thresholds were measured and found a statistically insignificant improvement after training, except in the strong eye (orthogonal/push-pull) (FIG. 4b). These findings indicate alterations of monocular efficiency [factors (i) and (ii)] are unlikely to significantly contribute to learning (reduced SED). Instead, they suggest the learning with the push-pull protocol is attributable to the activation of interocular inhibition whereby the weak eye suppresses the strong eye during training (“pull”). That is, repeatedly stimulating the putative inhibitory mechanism leads to perceptual learning.

The third sets of tests investigated whether reducing SED is beneficial for binocular depth processing. Binocular disparity threshold and reaction time was measured to detect the depth of a disc in a random-dot stereogram at the trained and untrained locations. It was found that depth threshold reduces significantly at the push-pull [t(6)=5.354, p=0.002] but not the push-only [t(6)=1.294; p=0.243] location (FIG. 4c), with a significantly larger reduction in the former [t(6)=2.824, p=0.030]. Reaction times to detect depth are reduced significantly at the push-pull [t(6)=3.104, p=0.021] but insignificantly at the push-only location [t(6)=2.086, p=0.082]. However, the pre and post-reaction time difference does not reveal a statistically significant effect of training protocol [t(6)=1.600, p=0.161)]. At the untrained locations (>1.53° from the trained location), there are no reliable changes in depth threshold [t(4)=−1.712, p=0.162] and reaction time [t(4)=−0.055, p=0.958]. Effectively, stereopsis is improved as a consequence of the push-pull protocol that aims at re-balancing interocular inhibition. Such a learning effect on stereopsis is particularly significant, as the training stimuli carried no binocular disparity and the observers were never trained on the stereo task.

### Example 2

### Push/Pull Protocol without Top-Down Focal Attention

A. Experimental Procedures

A Macintosh computer running MATLAB and Psychophysics Toolbox generated the stimuli on a flat-screen CRT monitor (1280×1024 pixels @ 100 Hz). A minor haploscopic system attached to a chin-and-head rest aided fusion from a viewing distance of 85 cm.

Six naïve observers (ages 27-35) with clinically normal binocular vision and informed consent were tested. All observers had normal or corrected-to-normal visual acuity (at least 20/20), clinically acceptable fixation disparity (≦8.6 arc min), central stereopsis (≦20 arc sec), and passed the Keystone vision-screening test.

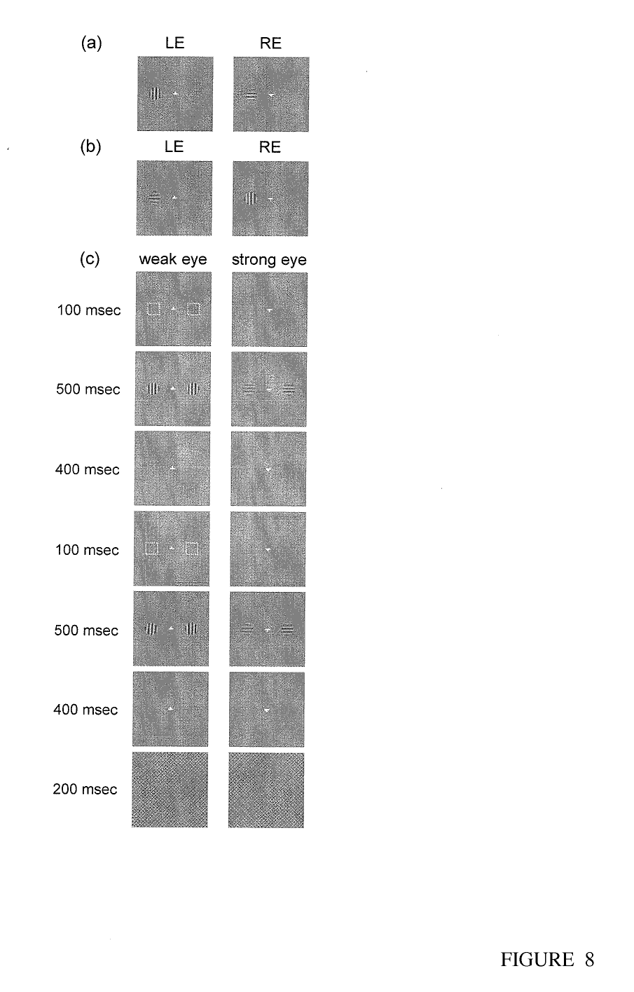

Local SED was measured with dichoptic vertical and horizontal grating discs (1.25° at eight concentric retinal locations 2° from the fovea (0°, 45°, 90°, 135°, 180°, 225°, 270°, and) 315°. Two locations with the largest SED were chosen for the training, one for the attended and the other for the unattended condition (the two locations had 4° spatial separation for four observers and 2.8° separation for two observers). During the 10-day Push-Pull training phase, two pairs of orthogonal grating discs (vertical/horizontal) simultaneously stimulated these two retinal locations (FIG. 8c). While both retinal locations received the same sequence of stimulation (cue, stimulus-1, cue, stimulus-2, mask), the observers were instructed to only attend to one location. They discriminated the grating orientation of the stimuli at the attended location (vertical vs. near-vertical), and ignored the stimulation at the unattended location. SED at the training locations were measured before each day's training session to monitor the learning progress. To further assess the learning effect, the following measurements were made at the two training locations in the pre- and post-training phases: boundary contour (BC)-based SED, dynamics of interocular dominance and suppression, stereo threshold and monocular contrast thresholds.

During the testing, measurements were taken to detect changes in interocular imbalance, boundary contour (BC)-based SEC, the dynamics of interocular dominance and suppression, stereo threshold, and monocular contrast threshold. Interocular imbalance was measured at 8 different retinal locations to evaluate the baseline and any change in SED. The stimulus comprised a pair of dichoptic vertical and horizontal sinusoidal grating discs (3 cpd, 1.25°, 35 cd/m²) (FIGS. 8a and 8b). The contrast of the horizontal grating was fixed (1.5 log unit) while the contrast of the vertical grating was varied (0-1.99 log unit). A trial began with central fixation on the nonius target (0.45°×0.45°, line width=0.1°, 70 cd/m²), followed by the presentation of the dichoptic orthogonal grating discs (500 msec), and terminated with a 200 msec mask (7.5°×7.5° checkerboard sinusoidal grating, 3 cpd, 35 cd/m², 1.5 log unit). The observer responded to his/her percept, vertical or horizontal, by key presses. If a mixture of vertical and horizontal orientation was seen, the observer would respond to the predominant orientation seen. The vertical grating contrast was adjusted after each trial using the QUEST procedure (50 trials/block) until the observer obtained equal chance of seeing the vertical and horizontal gratings, i.e., the point of neutrality. Each block was repeated twice. When the vertical grating was presented to the LE its contrast at neutrality is referred to as the LE's balance contrast. The grating discs were then switched between the eyes to obtain the RE's balance contrast. The difference between the LE and RE balance contrast is the SED.

In the pre-training phase, SED was measured at eight concentric retinal locations (0°, 45°, 90°, 135°, 180°, 225°, 270°, and 315°) 2° from the fovea. Thus, a total of 16 stimulus combinations (8 locations×2 eyes), in a randomized testing order, were run. From the eight retinal locations tested, two locations with the largest SED (˜0.3-0.4 log unit) were chosen for the training. During the training-phase, the SED at the two training-locations were measured with horizontal and vertical gratings before each day's training session.

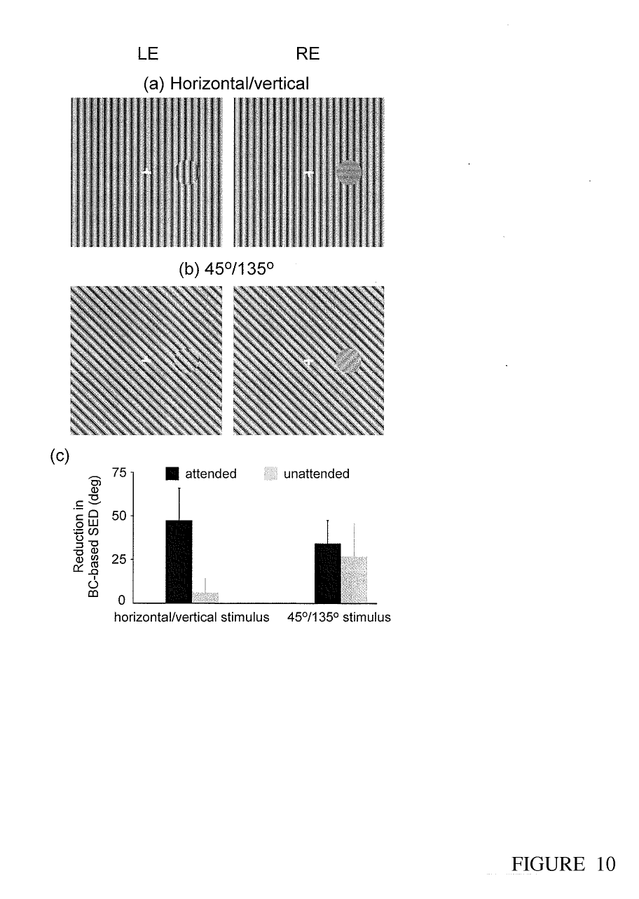

For boundary contour (BC)-based SEC testing, the stimulus comprised a pair of dichoptic vertical (1.8 log unit) and horizontal (1.2 log unit) sinusoidal grating discs (3 cpd, 1.25°, 35 cd/m²), each surrounded by vertical grating (3 cpd, 7.5°×7.5°, 1.8 log unit, 35 cd/m²) (FIG. 10a). The disc with the vertical grating in one half-image had a variable phase-shift (0-180 degrees) relative to the larger vertical grating surround. A trial began with central fixation on the nonius target (0.45°×0.45°, line width=0.1°, 70 cd/m²) and the presentation of the dichoptic stimulus (500 msec), followed by a 200 msec mask (7.5°×7.5° checkerboard sinusoidal grating, 3 cpd, 35 cd/m², 1.8 log unit). The observer responded to his/her percept, vertical or horizontal, by key presses. If a mixture of vertical and horizontal orientation was seen, the observer would respond to the predominant orientation seen. The relative phase-shift of the vertical grating disc was adjusted after each trial (step size=14 degree phase-shift) using the staircase procedure until the observer obtained an equal chance of seeing the vertical and horizontal gratings, i.e., the point of neutrality. Each block of trials (˜50-60 trials) comprised 30 reversals, with the last 26 reversals taken as the average threshold. When the vertical grating disc was presented to the LE its phase-shift at the point of neutrality is referred to as the LE's balance phase-shift. The grating half-images were then switched between the eyes to obtain the RE's balance phase-shift. The difference in the balance phase-shift between the LE and RE defines the BC-based SED. 4 stimulus combinations were tested [2 locations (attended+unattended)×2 eyes]. Each combination was repeated twice. The order of testing was randomized.

Separately, the BC-based SED was tested using 45° (1.2 log unit) and 135° (1.8 log unit) grating discs (1.25°, 3 cpd, 35 cd/m², 500 msec), each surrounded by 135° grating (3 cpd, 7.5°×7.5°, 1.8 log unit, 35 cd/m²) (FIG. 10b). The staircase method was used, and the phase-shift of the 135° grating disc relative to the 135° surround grating was adjusted after each trial (step size=14 degree phase-shift) until the point of neutrality was obtained for each eye.

The change in dynamics of interocular dominance and suppression was measured using a stimulus comprising a pair of dichoptic vertical and horizontal grating discs (1.25°, 3 cpd, 35 cd/m², 1.5 log unit) surrounded by a 7.5°×7.5° gray square (35 cd/m²) (similar to FIGS. 8a and 8b). A trial began with central fixation on the nonius target (0.45°×0.45°, line width=0.1°, 70 cd/m²) and the presentation of the dichoptic orthogonal gratings (30 sec), followed by a 1 sec mask (7.5°×7.5° checkerboard sinusoidal grating, 3 cpd, 35 cd/m², 1.5 log unit). The observer's task was to report (track) his/her instantaneous percept of the binocular competitive stimulus over the 30 sec stimulus presentation duration. Depending on the percept, vertical, horizontal, or a mixture of both, he/she would depress the appropriate key until the next percept took over.

Two grating orientation conditions were conducted: “same grating” vs. “orthogonal grating”. The same grating condition had the stimulus grating orientation presented to each eye been the same as the trained grating orientation. The orthogonal grating condition had the grating orientation switched between the two eyes. Altogether, there were 4 stimulus combinations [2 locations (attended+unattended)×2 conditions (same+orthogonal)]. Each combination was repeated 10 times in a randomized order.

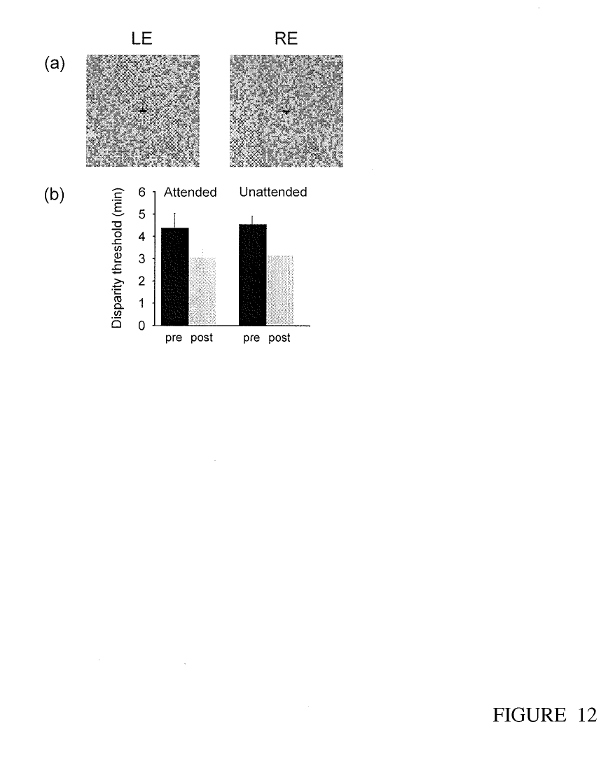

The stereo threshold was measured using a 7.5°×7.5° random-dot stereogram (dot size=0.0132°, 35 cd/m²) with a variable crossed-disparity disc target (1.25°) was used (FIG. 12a). The contrast of the stereogram was individually selected for each observer, to make the stereo task moderately difficult and to avoid a possible ceiling-effect due to pixel-size constraint. With this criterion, the contrast levels were set at 1.1 log unit for one observer, 1.2 log unit for 3 observers, and 1.3 and 1.5 log units, respectively, for the remaining two observers.

The standard 2AFC method was used in combination with the staircase procedure to measure stereo disparity threshold. The temporal sequence of stimulus presentation was fixation, interval-1 (200 msec), blank (400 msec), interval-2 (200 msec), blank (400 msec), and random-dot mask (200 msec, 7.5°×7.5°, 35 cd/m²). The observer indicated if the crossed-disparity disc was perceived in interval-1 or -2, and an audio feedback was given. Each block comprised 10 reversals (step size=0.8 arc min, total ˜50-60 trials), and the average of the last 8 reversals were taken as the threshold. Each block was repeated 4 times, and measured over two days. The order of testing was “ABBA” for day-1 and “BAAB” for day-2 (“A”=attended condition and “B”=unattended condition).

For monocular contrast threshold testing, the monocular sinusoidal grating (1.25°, 3 cpd, 35 cd/m², 500 msec) was either horizontal or vertical for the contrast sensitivity test. The fellow eye viewed a homogeneous field. The test was conducted using a 2AFC method in combination with the QUEST procedure. The 2AFC stimulus presentation sequence was: fixation, interval-1 (500 msec), blank (400 msec), interval-2 (500 msec), blank (400 msec), and mask (7.5°×7.5° checkerboard sinusoidal grating, 3 cpd, 35 cd/m², 1.5 log unit, 200 msec). The grating was presented at only one interval while the other interval was blank. The observer responded to seeing the grating either in interval-1 or -2 by key press, and an audio feedback was given. The grating contrast was adjusted after each trial (by QUEST) to obtain the threshold. 8 stimulus combinations were tested [2 locations (attended+unattended)×2 conditions (same+orthogonal)×2 eyes] in a randomized order. Each stimulus combination was repeated over 2 blocks of trials (50 trials/block).

B. Push-Pull Training Protocol at the Attended and Unattended Retinal Locations

The two retinal locations chosen for training were randomly assigned to the attended and unattended conditions, which were implemented simultaneously (FIG. 8c). A trial began with fixation at the nonius target. Then, at each retinal location, a transient attention cue (1.25°×1.25° frame with dash outline, width=0.1°, 1.56 log unit, 70 cd/m²) was presented monocularly to the weak eye for 100 msec. After a 100 msec cue-lead-time, a pair of dichoptic horizontal and vertical gratings (500 msec, 1.25°, 3 cpd, 35 cd/m²) was presented. The same 100 msec cue was presented again 400 msec later, followed by a 100 msec cue-lead-time, and the presentation of a second pair of dichoptic gratings (500 msec). The grating orientation shown to the weak eye in this second presentation had a slightly different orientation from the grating in the first presentation. Four hundred msec after the dichoptic grating presentation a binocular checkerboard sinusoidal grating mask (200 msec, 7.5°×7.5°, 3 cpd, 35 cd/m², 1.5 log unit) terminated the trial. The contrast values of the dichoptic gratings were those that led to the points of neutrality in the RE and LE with the interocular imbalance test. During the trial, the observer was instructed to attend only to one retinal location (attended condition) and ignore the stimulation at the other location (unattended condition).

Before commencing the proper training phase, it was determined for each observer whether the cue successfully suppressed the grating viewed by the strong eye. For the stimulation at the attended location, the observer reported by key press whether the first or second interval's grating had a slight counterclockwise orientation, and an audio feedback was given. Fifty such trials were run for each experimental block to obtain the orientation discrimination threshold using the QUEST procedure. Twelve blocks were performed during each training day.

C. Results

1. Reduction in SED at Both the Attended and Unattended Locations with the Trained Stimulus Feature

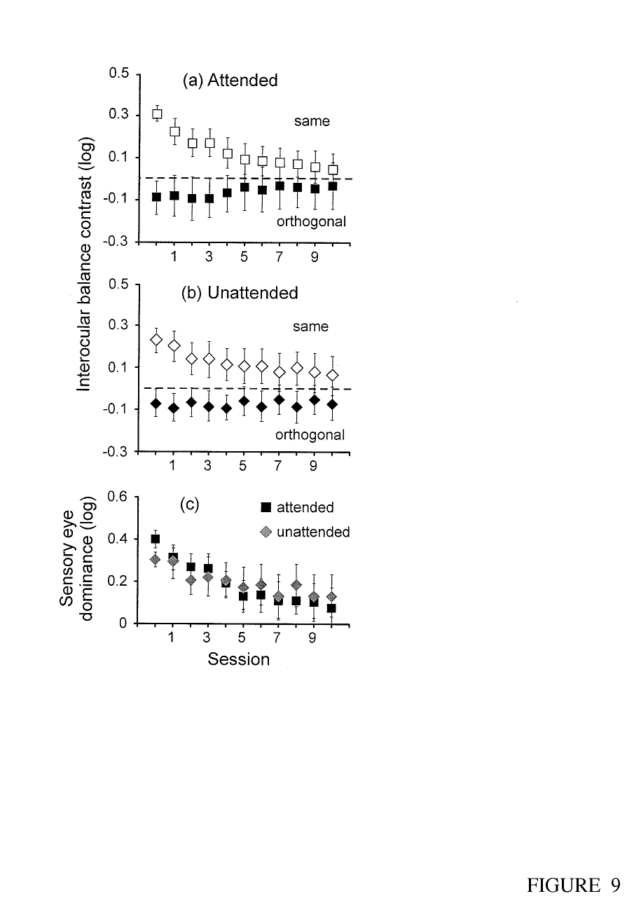

The balance contrast was tested with dichoptic gratings whose orientation in each eye was either the same as, or orthogonal to, the orientation of the grating used during the training. To be succinct, the former stimulation is called the “same grating” and the latter the “orthogonal grating”. FIGS. 9a and 9b plot the interocular balance contrast, which is defined as the difference between the measured balance contrast and 1.5 log unit (contrast of the fixed grating). With the same grating, the mean interocular balance contrast at the attended location (open squares, FIG. 9a) declines toward the balance point (horizontal dashed line) as the training progresses [slope=−0.0232, R²=0.8683, p<0.001]. But with the orthogonal grating at the attended location, the mean interocular balance contrast (filled squares, FIG. 9a) only tends slightly toward the balance point [slope=0.0068, R²=0.7749, p<0.001] with a much flatter slope [the interaction effect of 2-orientation vs. 11-training session: F(10, 50)=9.742, p<0.001, 2-way ANOVA with repeated measures]. This finding confirms that the learning effect is orientation and eye specific, i.e., taps on early cortical plasticity.

A similar learning effect was found at the unattended location. The mean interocular balance contrast with the same grating reduces toward the balance point with training (open diamonds, FIG. 9b) (slope=−0.0146, R²=0.8544, p<0.001). But the mean interocular balance contrast with the orthogonal grating only shows a weak tendency toward the balance point (slope=0.0016, R²=0.133, p=0.270) [interaction effect of 2-orientation vs. 11-training session: F(10, 50)=3.553, p=0.001, 2-way ANOVA with repeated measures].

FIG. 9c plots the SED, i.e., the difference between the same grating and orthogonal grating interocular balance contrast values obtained above. Clearly, SED reduces with training at both the attended (slope=−0.0300, R²=0.8968, p<0.001) and unattended locations (slope=−0.0162, R²=0.8136, p<0.001). The slope of the attended condition is significantly steeper than the slope of the unattended condition [F(10, 50)=3.961, p=0.001, 2-way ANOVA with repeated measures]. Altogether, these results reveal SED is significantly reduced at the unattended training location beyond the focus of top-down attention. However, top-down focal attention acts to facilitate perceptual learning as evidenced by the finding at the attended condition.

2. Reduction in Boundary Contour (BC)-Based SED Only at the Attended Location

The grating disc stimuli in FIGS. 10a and 10b have similar boundary contour (BC) strength (saliency of the circular disc outline enclosing the grating texture) in each half-image. Thus, the SED obtained from changing the relative grating contrast between the RE and LE mainly reflects the feature-based aspect of SED. It was measured if the SED reduction is associated with a change in the processing of the BC information, which can also affect SED. A BC-based SED test (FIG. 10a) was used, where the BC strength of the vertical grating disc is varied by changing the relative phase-shift between the vertical grating disc and the surrounding vertical grating. Meanwhile, the relative contrast of the dichoptic gratings remains constant. Doing so obtained the balance phase-shift, i.e., the point of neutrality between the two eyes. Thee balance phase-shifts was measured before and after the 10-day training period. If the weak eye strengthens after the training and leads to a reduction in BC-based SED, the phase-shift required to reach the point of neutrality will be smaller after, than before, the training.

FIG. 10c plots the reduction in BC-based SED, defined as the difference in the amount of phase-shift to reach the point of neutrality before and after training. Thus, a larger angular reduction in phase-shift indicates a larger reduction in BC-based SED. The leftmost bar shows the BC-based SED is significantly reduced at the attended location after the training [t(5)=2.571, p=0.050]. But it decreases little at the unattended retinal location (second bar) [t(5)=0.722, p=0.503]. Comparison between the two locations reveals the reduction in the mean BC-based SED at the attended location is significantly larger [t(5)=3.332, p=0.021]. This result suggests top-down focal attention plays a larger role in perceptual learning of the BC-based mechanism in SED.

A control condition was tested wherein the dichoptic test stimuli comprised 45° and 135° oriented gratings (FIG. 10b). Should the learning effect found in FIG. 10a be contributed by an enhanced BC strength in the weak eye (besides enhanced orientation feature), a similar learning effect with test stimuli whose grating orientations are different from the trained orientations would be expected. Confirming this, the result in FIG. 10c (two right bars) shows a significant reduction in the BC-based SED at the attended location [t(5)=2.601, p=0.048] and an insignificant reduction at the unattended location [t(5)=1.398, p=0.221]. Comparison between the two locations, however, does not reveal a significant difference [t(5)=0.289, p=0.784]. This finding of a learning effect only at the attended location may be attributed to the fact that the BC-based SED is partially mediated by the border ownership selective neurons in the extrastriate cortices (V2 and beyond), which receive robust top-down attention modulation.

3. Learning Effect on the Dynamics of Interocular Dominance and Suppression: Advantage at the Attended Location with the Trained Stimulus Feature

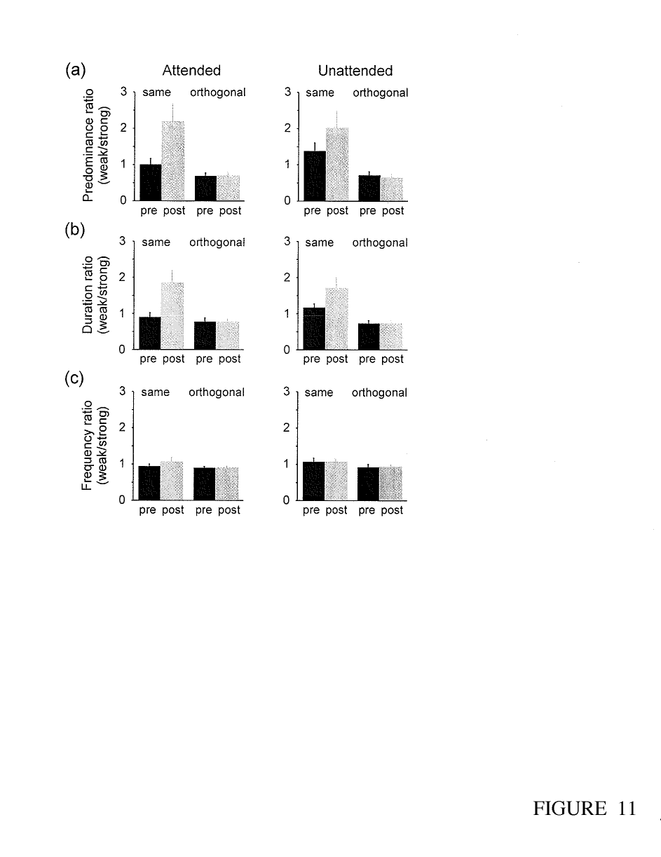

To reveal how the training influences the maintenance of perceptual dominance and its switching frequency, the observers tracked their perceptual dominance while viewing the binocular competitive stimulus (FIGS. 8a and 8b) over an extended duration (30 sec). The grating orientation stimulating the weak (trained) eye was either the same as, or orthogonal to, that during the training. From the data, the predominance, dominance duration and frequency of dominance was calculated. The graphs in the left and right panels of FIG. 11, respectively, for the attended and unattended conditions, present the mean ratios of the performance of the weak eye to that of the strong eye. A ratio of unity indicates the two eyes performed equally, while a ratio of greater than unity indicates the weak eye performed better for the given stimulus. FIG. 11a shows for each condition, the predominance ratio with the same grating stimulus is increased after the training, but do not change much with the orthogonal grating stimulus [F(1,5)=10.991, p=0.021, 3-way ANOVA with repeated measures]. This reconfirms that the learning is specific to the stimulus feature and eye-of-origin. Comparison between the performance with the same grating stimulus reveals a larger predominance ratio in the attended than unattended condition [Main effect of training: F(1,5)=7.295, p=0.043; interaction effect: F(1,5)=6.814, p=0.048, 2-way ANOVA with repeated measures]. Further analysis reveals a significant increase in predominance ratio at the attended location [t(5)=2.786, p=0.039] and a moderate increase at the unattended location [t(5)=2.444, p=0.058]. But for the orthogonal grating stimulus, 2-way ANOVA fails to reveal a reliable impact of the training on the predominance ratio (p>0.3).

The mean dominance duration ratios in FIG. 11b exhibit a similar trend as the predominance ratios in FIG. 11a. The dominance duration ratio (weak eye/strong eye) increases after the training with the same grating stimulus, with the larger increase at the attended location [Main effect of training: F(1,5)=7.027, p=0.045; interaction effect between training location and session: F(1,5)=5.307, p=0.069, 2-way ANOVA with repeated measures]. Further analysis reveals a significant increase in the duration ratio at the attended location [t(5)=2.741, p=0.041], and a moderate increase in the ratio at the unattended location [t(5)=2.345, p=0.066] with training. The duration ratios do not change reliably with training with the orthogonal grating stimulus. (Notably, the tracking predominance and duration findings here minor those found with the interocular imbalance test for SED that uses a detection task. That is, the same eye has the advantage in both the tracking and detection tasks.)

The average dominance frequency ratios in FIG. 11c do not show any learning effect. A 3-way ANOVA with repeated measures analysis reveals no reliable change in the dominance frequency ratio after the training (p>0.25).

4. Perceptual Training Improves Stereo Acuity at Both the Attended and Unattended Locations

Binocular disparity thresholds in the pre- and post-training phases were measured, using a random dot stereogram (FIG. 12a; an untrained stimulus) at the attended and unattended locations. Similar reductions in stereo thresholds are found at both locations with training [Main effect of the training: F(1,5)=23.656, p=0.005; interaction effect: F(1,5)=0.010, p=0.926, 2-way ANOVA with repeated measures] (FIG. 12b).

5. Monocular Contrast Threshold: Reduction Unlikely Associated with Changes in SED

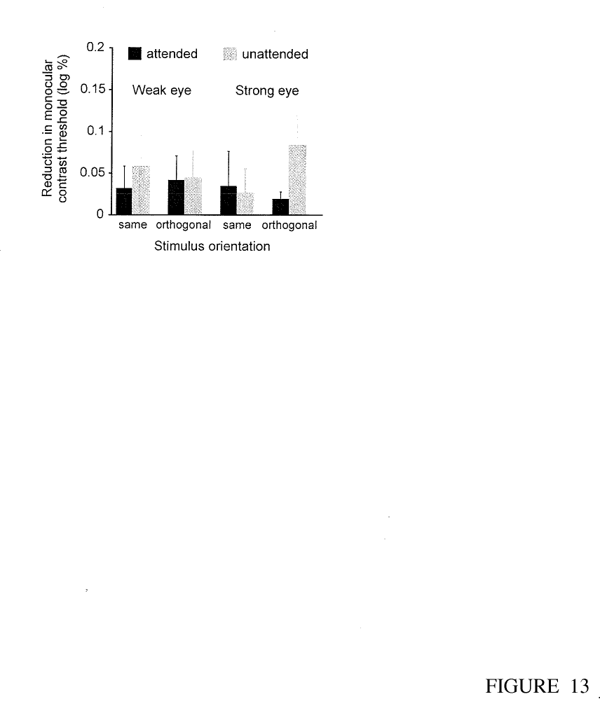

Monocular contrast thresholds were measured in the pre- and post-training phases with horizontal and vertical gratings. Small, but significant reductions in monocular contrast detection thresholds are found after the training at both locations (eye and stimulus) (FIG. 13) [Main effect of the training: F(1,23)=12.005, p=0.002; interaction effect: F(1,23)=1.609, p=0.217, 2-way ANOVA with repeated measures]. This generalized learning effect is unlikely to be associated with the reduction in SED. For example, had the reduction in monocular contrast thresholds been associated with SED reduction, the contrast threshold reduction in the weak eye would be larger than the contrast threshold reduction in the strong eye.

### Example 3

### Reduction of Foveal Sensory Eye Dominance within Early Visual Channels

A. Experimental Procedures

Eight naïve observers (23-33 years old) with informed consent and clinically normal binocular vision participated in the study. They had normal, or corrected to normal, visual acuity in each eye (at least 20/20), stereoacuity of ≦40 arc sec and fixation disparity of ≦8.6 arc min. During the experiments, they viewed the computer monitor through a haploscopic mirror system attached to a head-and-chin rest from a distance of 85 cm.

A MacPro computer running Matlab and Psychophysics Toolbox generated the stimuli that were displayed on a 21-inch Samsung SyncMaster flat screen CRT monitor. The monitor's resolution was set to 1280×1024 pixels at 100 Hz refresh rate for all experiments, except for the stereo threshold experiment (2048×1536 pixels at 60 Hz).

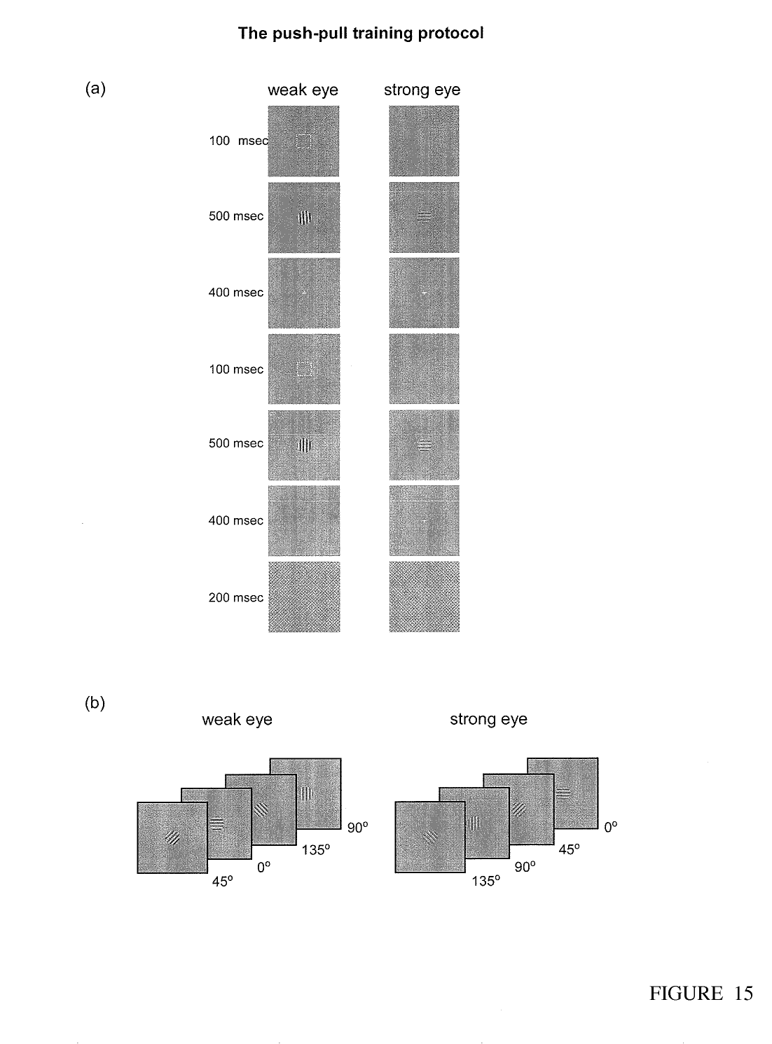

Foveal SED was measured (a) SED at the trained stimulus orientation and (b) SED with an untrained stimulus property in the pre- and post-training phases. With regard to the former, in the pre- and post-training test phase, SED was measured at random order at four orientations (0°, 45°, 90° and 135°) using the four pairs of binocular rivalry stimuli [2 eyes (left and right)×2 orientation pairs (0°/90° and)45°/135°] shown in FIG. 15b. (These same stimuli were also used for the push-pull training.) To prepare for a SED test trial, the observer aligned his/her eyes by fixating centrally on a nonius target (0.45°×0.45°, line width=0.1°, 52.5 cd/m²). He/she then pressed the spacebar on the computer keyboard to remove the nonius target. This was followed by the presentation of the dichoptic orthogonal grating discs (500 msec), and a 200 msec mask (7.5°×7.5° checkerboard sinusoidal grating, 3 cpd, 35 cd/m², 1.5 log unit contrast) to terminate the trial. The observer responded to his/her percept, 0° or 90° for the 0°/90° grating stimulus, or 45° or 135° for the 45°/135° grating stimulus, by pressing the appropriate key. If he/she saw a mixture of the two gratings, he/she would respond to the predominant orientation perceived. A QUEST procedure was used to adjust the grating contrast in one half-image according to the observer's response (the grating contrast in the other half-image was kept constant at 1.5 log unit). By appropriately adjusting the grating contrast after each trial, the point of equality, where the observer obtained an equal chance of seeing the two gratings (equal predominance) was reached. The obtained contrast is the balance contrast for the eye that viewed the variable contrast grating. The gratings were switched between the two eyes, to obtain the mean balance contrast for the fellow eye. Two such blocks of trials (50 trials/block) were run to obtain the mean balance contrast for each eye. The difference between the LE and RE mean balance contrast values is defined as the SED. For convention and ease of referencing, if the contrast of the 0° (horizontal) grating of the 0°/90° grating stimulus was adjusted, the obtained SED is labeled as 0°-0°/90° SED. (That is, the first number is the orientation of the grating with the variable contrast while the second and third numbers indicate the orientations of the dichoptic grating stimulus used in the SED test.)

During the training phase, the 0°-0°/90° SED and 45°-45°/135° SED before and after each even day's training session was measured. Meanwhile, the 90°-0°/90° SED and 135°-45°/135° SED were measured before and after each odd day's training session.

SED with an untrained stimulus property in the pre- and post-training phases was measured with binocular rivalry stimuli whose spatial properties were the same as the training stimuli in all aspects except one (contrast, orientation or spatial frequency). Specifically, the following were tested:
i. 0°-0°/90° SED and 45°-45°/135° SED at two different fixed contrast levels: 1.3 log and 1.7 log units. (The standard SED test stimulus had the grating of one half-image with a variable contrast level, while the contrast of the grating in the other half-image was kept constant at 1.5 log unit. The training was also carried out with one half-image of the training stimulus having a contrast level of 1.5 log unit.)
ii. 22.5°-22.5°/112.5° SED and 67.5°-67.5°/157.5° SED, i.e., 22.5° away from the trained orientations.
iii. 0°-0°/90° SED and 45°-45°/135° SED at 6 cpd, i.e., 1 octave higher than the trained spatial frequency (3 cpd).

During testing, the dynamics of interocular dominance and suppression and stereothreshold were tested. With the former, the stimulus was the same as the 0°/90° binocular rivalry stimulus used to measure SED, except for the stimulus presentation duration. Specifically, it comprised a pair of dichoptic vertical and horizontal grating discs (1.5°, 3 cpd, 35 cd/m², 1.5 log unit contrast) surrounded by a 7.5°×7.5° gray square (35 cd/m²). To begin a trial, the observer aligned his/her eyes on the nonius fixation target (0.45°×0.45°, line width=0.1°, 52.5 cd/m²), and then pressed the spacebar on the computer keyboard. This led to the removal of the nonius fixation target, which was replaced by the presentation of the binocular rivalry gratings for 30 sec. At the end of the 30 sec, a 1 sec mask (7.5°×7.5° checkerboard sinusoidal grating, 3 cpd, 35 cd/m², 1.5 log unit contrast) terminated the trial. The observer's task was to report (track) his/her instantaneous percept of the binocular rivalry stimulus over the 30 sec stimulus presentation duration. Depending on the percept, vertical, horizontal, or a mixture of both, he/she would depress the appropriate key until the next percept took over. The predominance, average duration and frequency of seeing these percepts were calculated. Two orientation-eye conditions (horizontal in weak eye and horizontal in strong eye) were run 5 times each in a randomized order.

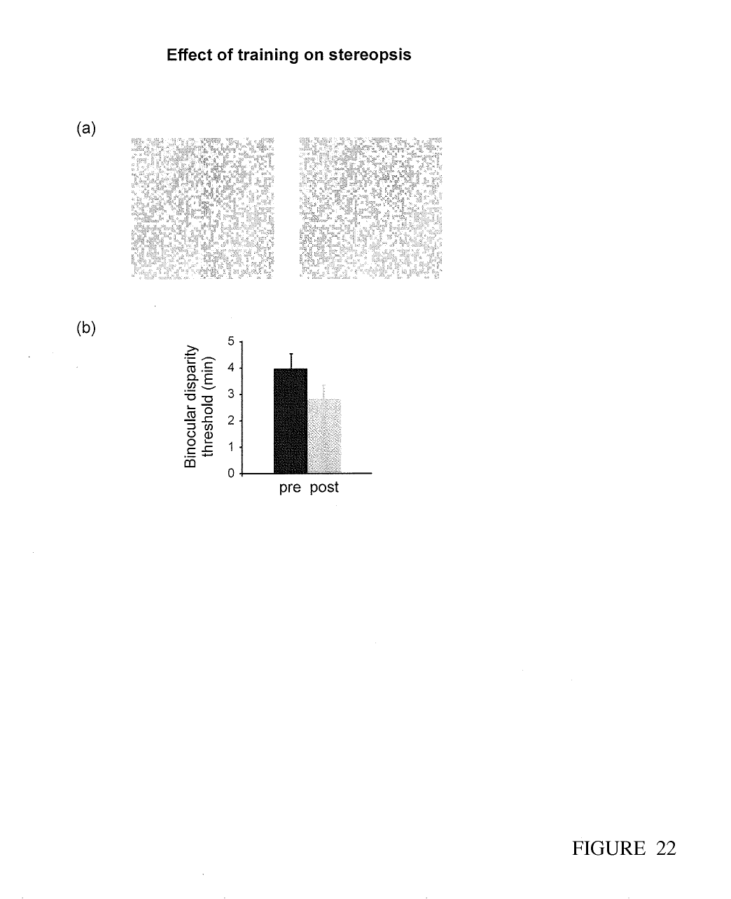

For Stereo threshold, a 7.5°×7.5° random-dot stereogram (dot size=0.0132°, 35 cd/m²) with a variable crossed-disparity disc target (1.5°) was used (FIG. 22a). The Michelson contrast of the stereogram was individually selected for the observer, to make the stereo task moderately difficult and to avoid a possible ceiling-effect due to pixel-size limitation (0.9 arc minute). With this criterion, the contrast levels were set at 1.0 log unit for two observers, 1.1 log unit for two observers, 1.2 log unit for two other observers, and 1.3 and 1.5 log unit, respectively, for the remaining two observers.

Standard 2AFC method was used in combination with the staircase procedure to measure the stereo disparity threshold. The temporal sequence of the stimulus presentation was interval-1 (200 msec), blank (400 msec), interval-2 (200 msec), blank (400 msec), and random-dot mask (200 msec, 7.5°×7.5°, 35 cd/m²). The observer indicated whether the crossed-disparity disc was perceived at interval-1 or -2, and an audio feedback was given. Each block comprised 10 reversals (step size=0.8 arc min, total ˜50-60 trials), and the last 8 reversals were taken as the average threshold. Each block was repeated four times and measured over two days.

To investigate whether the push-pull training has a long-term effect on stereo threshold, all, but one observer, were tested about 10 months or beyond after the termination of the training phase. One observer (whose stereo test contrast level was set at 1.1 log unit) had relocated and was not able to return to the laboratory. The remaining observers were able to return to the laboratory for a one-time testing, though not on the same day. Specifically, the remaining seven observers were tested on the 314^{th}, 318^{th}, 381^{st}, 402^{nd}, 459^{th}, 470^{th}, or 492^{nd} day after the training phase terminated.

B. The Push-Pull Training Protocol Stimulating the Foveal Region

To begin a training trial, the observer aligned his/her eyes at the nonius fixation target (0.45°×0.45°, line width=0.1°, 52.5 cd/m²), and then pressed the spacebar on the computer keyboard. This led to the removal of the nonius fixation target, which was replaced by the presentation of a monocular square frame (1.5°×1.5° frame with dash outline, width=0.1°, 1.52 log unit, 70 cd/m²) in the weak eye for 100 msec (FIG. 15a). The square frame acted as a transient attention cue to attract attention to the vicinity of the cue in the weak eye. After a 100 msec cue-lead-time, a pair of dichoptic orthogonal gratings (500 msec, 1.5°, 3 cpd, 35 cd/m²) was presented. The same 100 msec cue was presented again 400 msec later, followed by a 100 msec cue-lead-time, and the presentation of a second pair of dichoptic gratings (500 msec). The grating orientation shown to the weak eye in this second presentation had a slightly different orientation from the grating shown in the first presentation. Four hundred msec after the dichoptic grating presentation a binocular checkerboard sinusoidal grating mask (200 msec, 7.5°×7.5°, 3 cpd, 35 cd/m², 1.5 log unit contrast) was presented to terminate the trial. The contrast values of the dichoptic gratings used in the training were those that led to the points of equality in the RE and LE with the SED test obtained before the training phase. The observer's (orientation discrimination) task was to report by key press whether the first or second grating had a slight counterclockwise orientation, and an audio feedback was given. This ended a training trial.

Five hundred training trials were run during each day's training session. These trials were blocked into 100 trials/block, i.e., 5 blocks of trials were performed on each training day. Within each block of trials, four pairs of dichoptic training stimuli (FIG. 15b) were presented to the observer to train the weak eye at four different orientations (0°, 45°, 90°, and 135°). Trials with the four different orientations (25 trials per orientation) were intermingled and their order of presentation was randomized. Thus, to determine the orientation discrimination threshold for each stimulus orientation, four randomly interleaved QUEST procedures were run during each block of 100 trials. Before commencing the proper training phase, we ascertained for each observer that the cue successfully suppressed the grating viewed by the strong eye.

C. Results

1. Reduction in SED Measured with Stimuli Similar to the Trained Stimuli

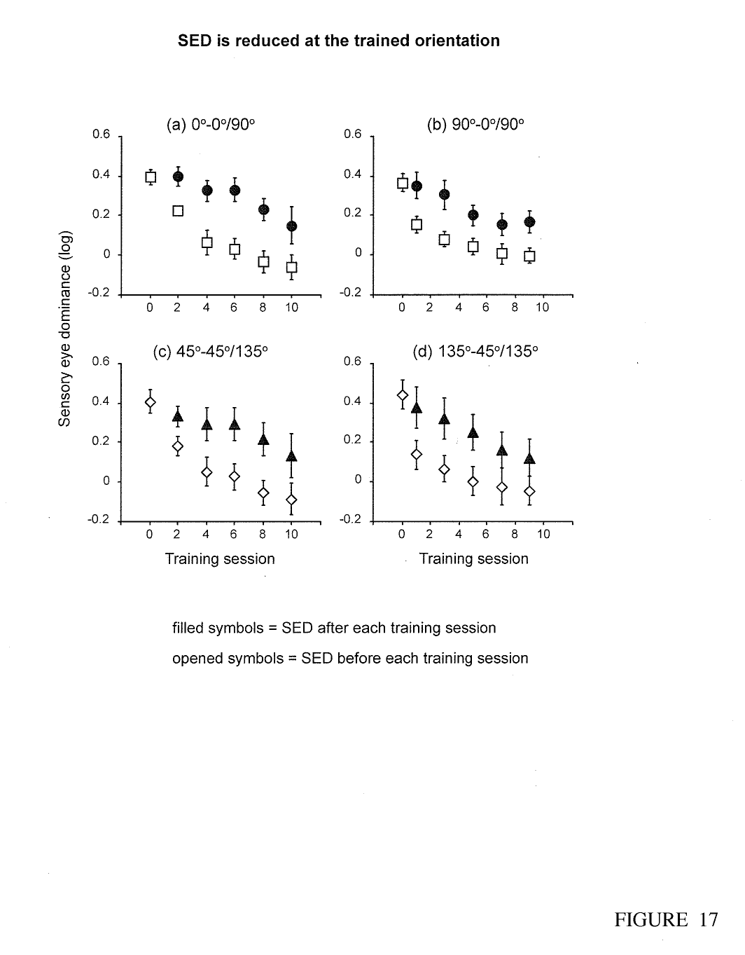

FIG. 17 depicts the average SED (0°-0°/90°; 90°-0°/90°; 45°-45°/135°; 135°-45°/135° as a function of training session, measured using SED test stimuli with the same spatial properties as the four pairs of training stimuli (FIG. 15b). The open and filled symbols, respectively, represent the average SED measured before and after each day's training session. For all four stimuli, the SED is smaller before than after the training session [0°-0°/90°: F(1,7)=22.061, p=0.002; 90°-0°/90°: F(1,7)=17.797, p=0.004; 45°-45°/135°: F(1,7)=27.981, p=0.001; 135°-45°/135°: F(1,7)=25.408, p=0.001; 2-way ANOVA with repeated measures]. The trend of this finding (within-session increase in SED) is similar to those found in the parafoveal region and those found previously for other aspects of perceptual learning. This (short-term) within-session increase in SED was monitored on three observers. Their SED was tested over an hour after the training session ended for the day. It was found that the short-term increase in SED decays slowly (e.g., the effect can still be observed 30 min after the training session ended), suggesting the possible contributions of cortical contrast adaptation, fatigue of the interocular inhibitory network, and/or general cognitive fatigue.

SED reduced gradually as the training progresses when it was measured either before (0°-0°/90°: slope=−0.044, R²=0.898, p=0.004; 90°-0°/90°: slope=−0.033, R²=0.803, p=0.016; 45°-45°/135°: slope=−0.046, R²=0.881, p=0.006; 135°-45°/135°: slope=−0.042, R²=0.768, p=0.022), or after each day's training session (0°-0°/90°: slope=−0.030, R²=0.928, p=0.008, 90°-0°/90°: slope=−0.025, R²=0.865, p=0.022; 45°-45°/135°: slope=−0.024, R²=0.902, p=0.013; 135°-45°/135°: slope=−0.033, R²=0.989, p=0.001). These observations thus demonstrate that the push-pull training protocol can significantly reduce SED in the foveal region, as in the parafoveal region.

2. Reduction in SED Measured with an Untrained Stimulus Property: Different Fixed Contrast Level

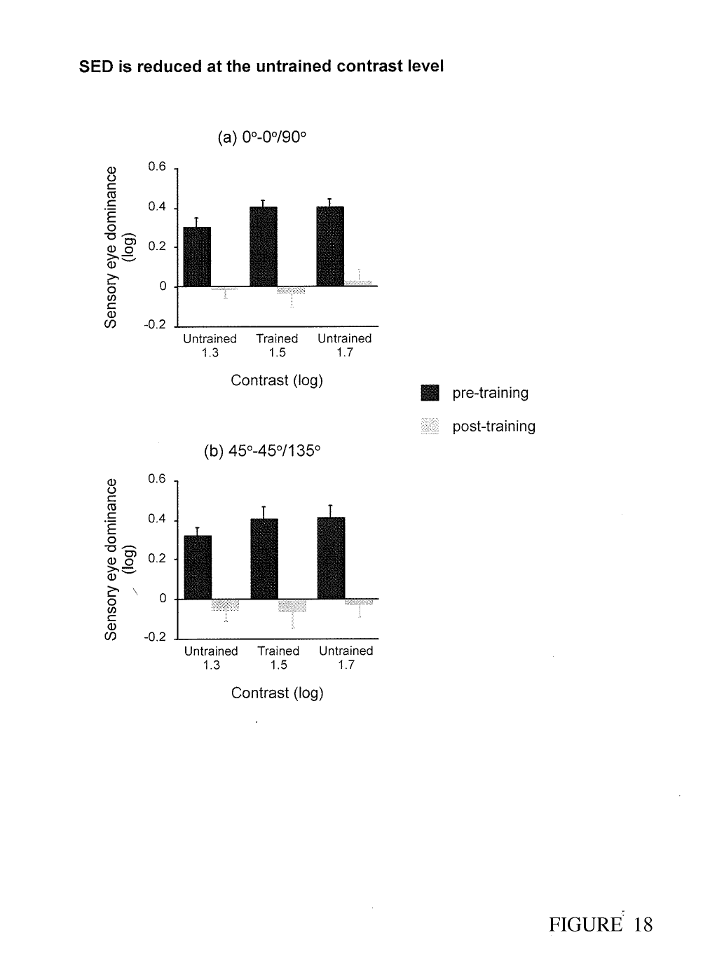

To investigate whether the learning effect occurs for a test stimulus contrast level different from that of the trained stimulus contrast level, 0°-0°/90° SED and 45°-45°/135° SED was measured with either a higher (1.7 log unit) or lower (1.3 log unit) fixed contrast level than that used in the training stimulus (1.5 log unit). The graph in FIG. 18a shows that the average 0°-0°/90° SED is significantly reduced after the training phase, when measured with either the lower and higher fixed contrast grating [1.3 log unit: t(7)=5.876, p=0.001; 1.7 log unit: t(5)=7.497, p=0.001]. [Note: Even though 8 observers were trained, 2 observers could not be tested with the 1.7 log unit fixed contrast for the 0°-0°/90° SED before the training (due to excessive SED) because the highest contrast level in the weak eye (˜2 log unit) could not balance out the fixed 1.7 log unit contrast in the strong eye. Therefore, only included 6 observers' data were included when averaging the 0°-0°/90° SED results.] The reduction in SED is similar at all three fixed contrast levels for all six observers tested [interaction effect between contrast level and session: F(2,10)=1.257, p=0.326, 2-way ANOVA with repeated measures]. A similar learning effect is revealed for the 45°-45°/135° SED [1.3 log unit: t(7)=9.680, p<0.001; 1.7 log unit: t(7)=7.386, p<0.001] (FIG. 18b). The reduction in SED is also similar for all three fixed contrast levels tested [interaction effect between contrast level and session: F(2,14)=2.856, p=0.091, 2-way ANOVA with repeated measures].

3. Reduction in SED Measured with an Untrained Stimulus Property: Different Orientation

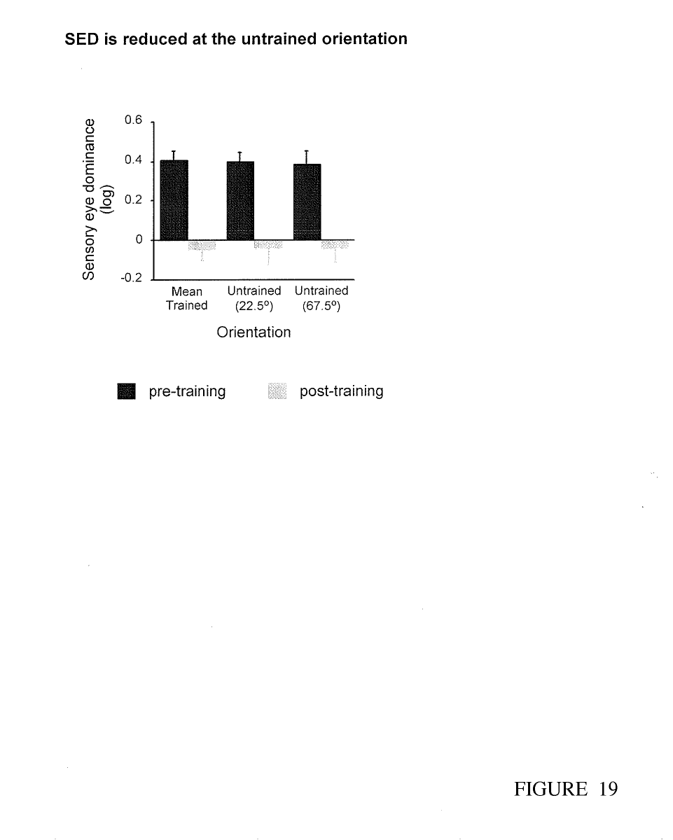

22.5°-22.5°/112.5° SED and 67.5°-67.5°/157.5° SED was measured, whose orientations are 22.5° away from the nearest trained orientation (FIG. 19). A significant reduction in the average SED is found after the training phase [22.5°-22.5°/112.5°: t(7)=5.802, p=0.001; 67.5°-67.5°/157.5°: t(7)=9.160, p<0.001], indicating a transfer of perceptual learning to the untrained orientations. To quantify the transfer effect from all four trained orientations, a transfer factor was calculated. This is defined as the ratio of the mean reduction in SED from the two untrained orientation conditions (22.5°-22.5°/112.5° and 67.5°-67.5°/157.5°) to the mean reduction in SED from all four trained orientations (see “Mean trained” in the graph). The transfer factor was found to be 99.63%. This suggests an almost complete transfer of the learning effect to the untrained orientations, when the untrained orientations are within the estimated bandwidth of the orientation tuning functions of the early visual cortex. Furthermore, since the untrained orientations fall in the mid-ranges of the two trained orientations, which are 45° apart, the results indicate that the four orientations of the training stimuli (0°, 45°, 90° and 135°) can produce a similar learning effect at any other untrained orientation.

4. Reduction in SED Measured with an Untrained Stimulus Property: Different Spatial Frequency

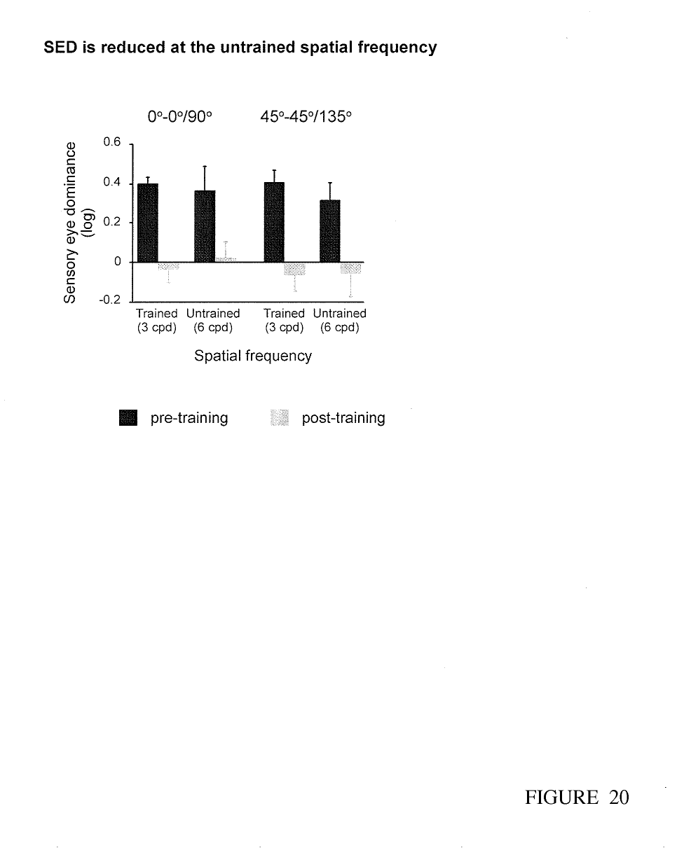

0°-0°/90° SED and 45°-45°/135° SED was measured using test stimuli with 6 cpd, instead of 3 cpd (trained spatial frequency). The untrained spatial frequency of 6 cpd is one octave higher than the trained grating's and is within the estimated bandwidth of the spatial frequency tuning function centered at 3 cpd. As shown in FIG. 20, the 0°-0°/90° SED and 45°-45°/135° SED were significantly reduced after the training [0°-0°/90°: t(7)=3.311, p=0.013; 45°-45°/135°: t(7)=2.661, p=0.032], indicating a transfer effect of perceptual learning to a different spatial frequency that is within the bandwidth of the same spatial frequency tuning function. The SED transfer factors were calculated from the trained to the untrained spatial frequency (ratio between the reduced SED at 6 cpd and reduced SED at 3 cpd). Relatively large transfer factors were found for both the 0°-0°/90° (77.04%) and 45°-45°/135° (70.77%) stimuli.

5. Significant Learning Effect on the Dynamics of Interocular Dominance and Suppression

Observers' performance in tracking their percepts (horizontal grating, vertical grating or mixture) were measured while viewing a pair of dichoptic horizontal/vertical rivalry gratings for 30 sec. There were two test conditions, one with the weak eye viewing the vertical grating and the other with the weak eye viewing the horizontal grating. For each test condition, the predominance, dominance duration, suppression duration, and dominance frequency were calculated for seeing horizontal and vertical gratings. The performance ratio between the strong eye (SE) and weak eye (WE) was calculated to quantify the binocular rivalry percept for each measure. In the case of predominance, for example, the performance ratio is obtained by the formula:

$$\frac{[ \begin{matrix}Predominance ( SE seeing vertical ) + \\ Predominance ( SE seeing horizontal )\end{matrix} ]}{[ \begin{matrix}Predominance ( WE seeing vertical ) + \\ Predominance ( WE seeing horizontal )\end{matrix} ]}$$

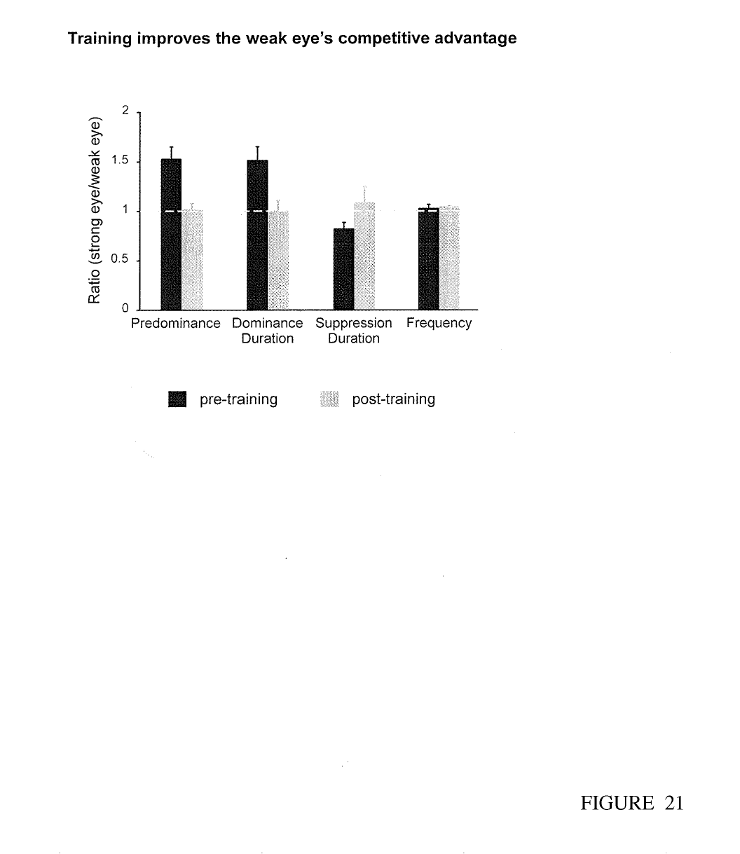

FIG. 21 depicts the average performance ratio results for the various measures of the binocular rivalry percepts. A performance ratio of larger than unity indicates an imbalance that favors the strong eye and a performance ratio of unity indicates the two eyes are balanced.

Clearly, all average performance ratios, except for the dominance frequency performance ratio, change significantly toward the balance level (ratio=1) after the training. Predominance: t(7)=7.073, p<0.001; Dominance duration: t(7)=9.339, p<0.001; Suppression duration: t(7)=−2.406, p=0.047; Dominance frequency: t(7)=−0.250, p=0.810. Additionally, the mixture (piecemeal) percept was analyzed but did not find a significant learning effect (p>0.250)].

As noted, other than the viewing duration, the same stimuli for measuring SED and the current binocular rivalry tracking test were used. The viewing duration for the SED test was 500 msec, which required the observers to quickly detect the appearance of the image seen. On the other hand, the viewing duration of the rivalry tracking task was 30 sec, which allowed the observers ample time to experience the alternation of their percepts between dominance and suppression. Nevertheless, despite the difference, both psychophysical tasks provide insights into the behaviors of the interocular inhibitory mechanism. Measuring SED reveals the interocular imbalance at the initial stage of interocular inhibition, while tracking the binocular rivalry percept largely reveals the interocular imbalance between the eyes as they compete to maintain dominance and emerge from suppression. Consequently, it was predicted that the two measures should be correlated such that the same eye would have the competitive advantage in both tasks. By extension, the learning effect should be evident in (translate to) both tasks. This prediction was confirmed.

6. Significant Learning Effect on Stereopsis: Reduction in Stereo Threshold

The stereo depth thresholds were measured with a random-dot stereogram and it was found that a significant threshold reduction exists after the training phase [t(7)=11.325, p<0.001] (FIG. 22). This finding is similar to those found after training the parafoveal region. Given that the observers were not exposed to the random-dot stereogram stimulus during the training phase, the observed learning effect on stereo depth perception also suggests that the push-pull training protocol modifies the binocular neural circuitries in the early cortical level. This could explain how a binocular perception (stereopsis) that is unrelated to the training stimulus, or task, improves.

Additionally, the stereo depth thresholds of seven of the eight observers' were tested more than 10 months after the push-pull training phase ended to investigate the retention of the stereo learning effect. These observers were found to exhibit an average stereo threshold of 2.99±0.31 min. Importantly, the stereo threshold level (2.99±0.31 min) is comparable with the average stereo threshold obtained immediately after the push-pull training phase ended [2.93±0.61 min, t(6)=0.096, p=0.926]. Each observer's stereo depth threshold is smaller than the one measured before the start of the push-pull training phase, and on average the former is moderately smaller than the latter [4.17±0.60 min; t(6)=2.064, p=0.085]. This suggests that the learning effect on stereopsis is retained for a relatively long period of time.

7. Reductions in the Weak Eye's Orientation Discrimination Thresholds During the Training Phase

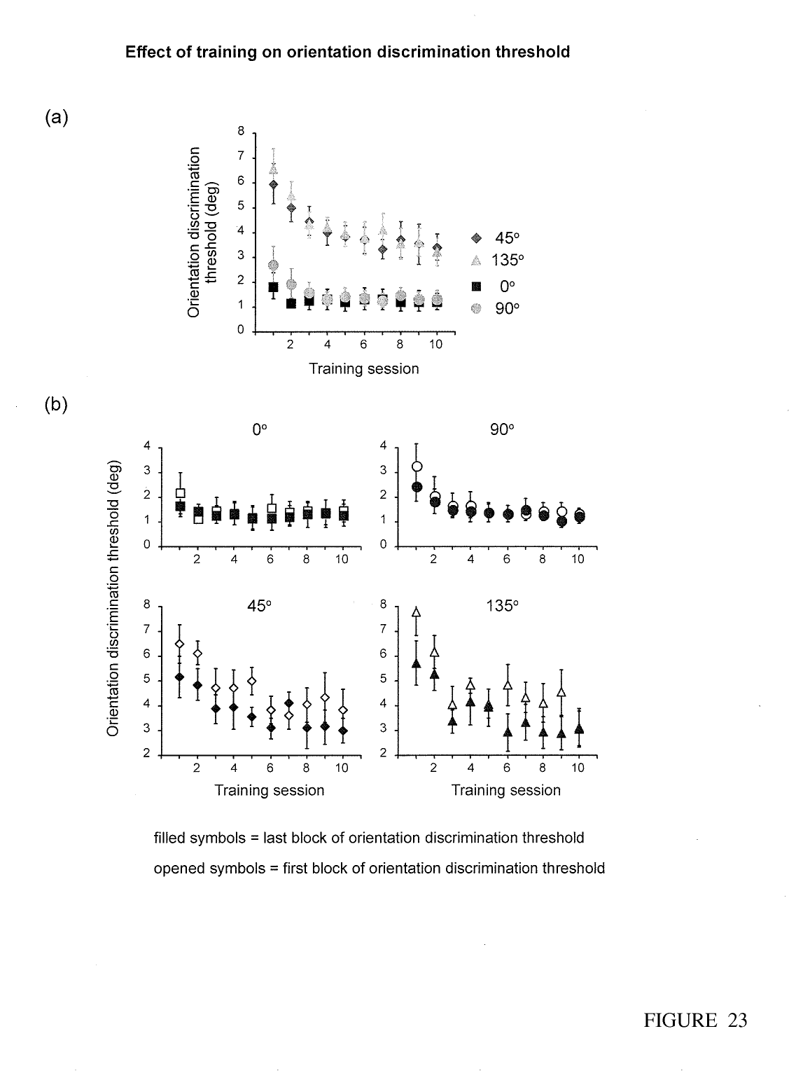

The observers' task during the push-pull training trials was to perform orientation discrimination of the gratings viewed by the weak eye. Essentially, while the goal was to reduce SED through training, orientation discrimination ability was also trained at four orientations (0°, 45°, 90° and 135°). To assess the training effect of orientation, the orientation discrimination thresholds were averaged from the 5 blocks of trials ran for each orientation during each training day (session). These are shown in FIG. 23a as a function of training session. There is a significant learning effect for all four orientations [0°: F(9,36)=2.829, p=0.007; 45°: F(9,36)=5.191, p<0.001; 90°: F(9,36)=6.085, p<0.001; 135°: F(9,36)=9.144, p<0.001, one-way ANOVA with repeated measures]. It is notable that the orientation discrimination thresholds) (˜6° for the oblique gratings found here are moderately higher compared to those typically reported in the literature. Whether the elevated orientation discrimination thresholds are related to the binocular rivalry condition inherent in the push-pull paradigm requires further investigations (previous studies did not use rivaling gratings).

Since 5 blocks of training trials were ran during each training session, the orientation discrimination thresholds were compared between the first and the last blocks of the session. This revealed the behavior of the learning process within each day's training session. The average results of the four orientations are depicted in FIG. 23b (first block: open symbol; last block: filled symbol). There is no significant difference in orientation discrimination thresholds between the first and last blocks for the horizontal and vertical orientations [0°: F(1,7)=1.525, p=0.257; 90°: F(1,7)=2.060, p=0.194; 2-way ANOVA with repeated measures]. However, there exists a significant difference in orientation discrimination thresholds between the first and last blocks for the oblique orientations [45°: F(1,7)=46.615, p<0.001; 135°: F(1,7)=22.690, p=0.002, 2-way ANOVA with repeated measures]. Interestingly, the improved performance in orientation discrimination within the day's training for the oblique orientations is opposite to the trend found in the SED measures. In the latter, SED is smaller (improved) before than after the end of the training session (FIG. 15). Taken together, this suggests that the change in SED (i.e., the short-term, within-session increase in SED) cannot solely be explained by a general fatigue effect that degrades all types of perceptual performance.

While not intended to be bound by theory, there are two possible explanations for the within-session increase in SED. First, as mentioned earlier, it could be caused by contrast adaptation in the early visual cortex. Specifically, the induced contrast adaptation during the training session (500 trials lasting about 1 hour) could be larger for the training grating presented to the weak eye than for the training grating presented to the strong eye. This is because the dominant grating (seen by the weak eye) is more susceptible to contrast adaptation than the suppressed grating (seen by the strong eye). Therefore, when SED is measured immediately after the training session, the weak eye's monocular channel being differentially more adapted would be disadvantaged. This is revealed as an increase in (within-session) SED.

However, the above monocular contrast adaptation explanation might not be the sole factor. Specifically, increases in within-session SED at both locations were found with the within-session SED increase being larger at the push-pull training location. Now, should monocular contrast adaptation be the sole factor, the within-session SED increase should instead be larger at the push-only training location. This is because the push-only training does not lead to an adaptation of the monocular strong eye channel since a grating is not presented to it during the training phase.

Consequently, a second possible explanation for the within-session increase in SED is that subjecting the strong eye to multiple and consecutive interocular inhibition by the weak eye during the hour long push-pull training phase leads to a fatigue in the underlying interocular inhibitory network. This, effectively, causes the short-term shift in the balance point of mutual interocular inhibition toward the strong eye. Hence, the within-session SED is increased.

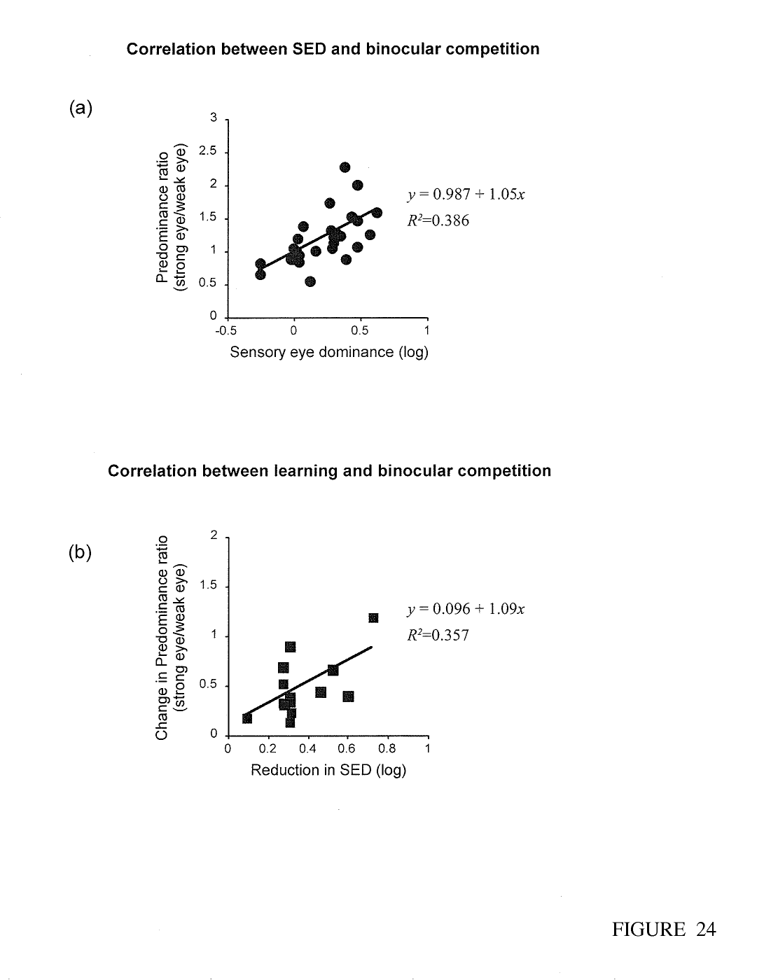

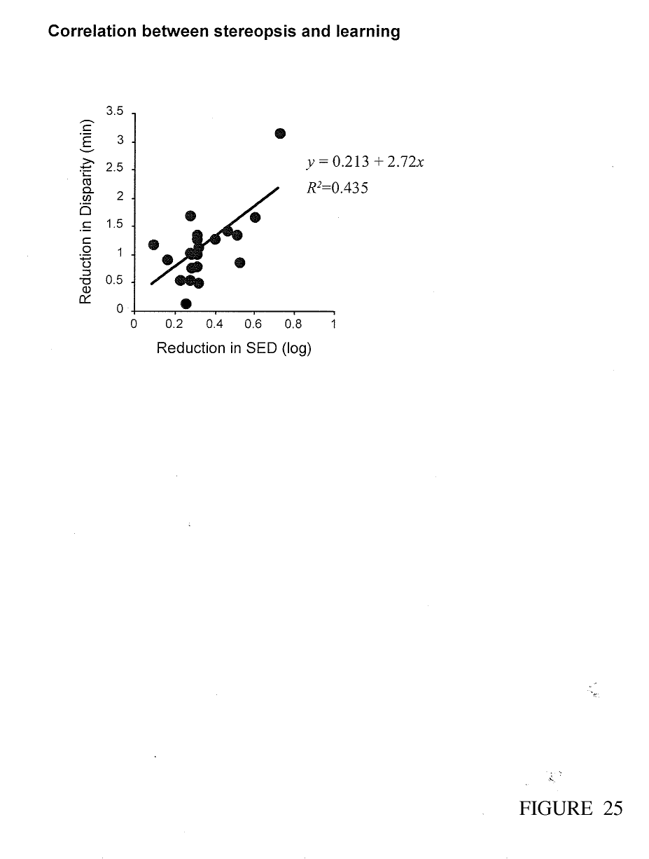

The above findings suggest that the push-pull training protocol largely affects the interocular inhibitory neural network residing in the primary visual cortex. Since interocular inhibition is an integral part of the binocular visual processing, it is not surprising that the learning gained from the push-pull training protocol extends to other binocular visual functions besides reduced SED. Consistent with this, the learning effect was revealed to extend to binocular rivalry with extended viewing duration and stereo perception (FIGS. 21 and 22). Along this line of thinking, a reliable correlation exists between these binocular functions and the learning effect. To evaluate this prediction for binocular rivalry, the foveal data was compared with the parafoveal data above, and each observer's predominance ratio (SE/WE) and SED were plotted in FIG. 24a. Clearly, these two measurements vary in the same direction (R²=0.386, p<0.001). Using the same data, the correlation coefficient between the change in the predominance ratio (pre-post training) and the reduction in SED after training was evaluated. As shown in FIG. 24b, a significant correlation was found between these two changes (R²=0.357, p=0.024), wherein observers with more reduction in SED have a larger change in their binocular rivalry perception. The relationship between the reduction in stereo disparity thresholds and reduction in SED was examined (FIG. 25), again using the data from the current experiment and those above. A significant correlation is found (R²=0.435, p=0.001), indicating observers whose binocularity became more balanced (reduced SED) also have more reduction in binocular disparity threshold (improved stereoacuity).

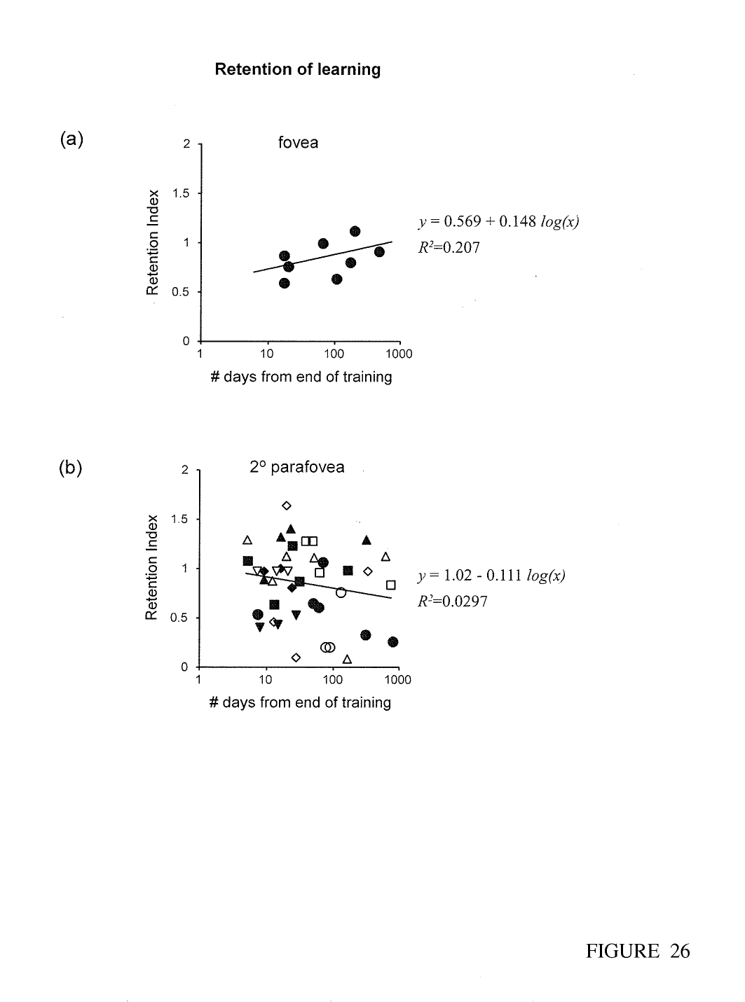

The learning effect on SED in the foveal (current study) and 2° parafoveal training locations above were observed to be retained for a relatively long period after the training phase ended, without an intervening re-training session. In the current study, observers returned to the laboratory (each on a different day) for SED testing at the four trained orientations (0°, 45°, 90°, and 135°). The SED data obtained with the four orientations were averaged and used for comparison with the averaged SED immediately after the training terminated. To quantify such a comparison (FIG. 26a) the Retention Index (RI) was calculated, which is defined by the following formula:

(SEDₚᵣₑ-SED_{long}_{_{—}}_{after})/(SEDₚᵣₑ-SEDₚₒₛₜ)

In the formula, SEDₚᵣₑ is the SED measured before the training phase began, SEDₚₒₛₜ is the SED measured immediately after the training phase ended and SED_{long}_{_{—}}_{after} is the SED measured some intervals after the training phase ended. Thus, an RI of unity indicates the learning effect is fully retained at the time the SED_{long}_{_{—}}_{after} is conducted, and an RI of zero means the learning effect has dissipated at the time the SED_{long}_{_{—}}_{after} is conducted. FIG. 26a shows that all eight observers' RI (filled circles) is above 0.5, indicating the learning effect at the fovea can be retained for quite a long period. The 2° parafoveal SED data above was also analyzed in a similar manner and were plotted in FIG. 26b. Here, each of the ten observers' data points are plotted with different symbols to reflect his/her individual SED changes over the intervals measured (the number of data points are not equal because some observers were able to return to the laboratory more times than others). Overall, all observers' RI are above zero with a mean around unity, indicating the retention of learning.

### Example 4

### Further Support for the Importance of the Suppressive Signal (Pull) During the Push-Pull Perceptual Training

A. Experimental Procedures

The stimuli were presented on a flat-screen CRT monitor (2048×1536 pixels @ 75 Hz, except for the contrast-SED test with 1280×1024 pixels @ 100 Hz) using a MacPro computer running MATLAB and Psychophysics Toolbox. The two half-images were viewed through a minor haploscopic system attached to a chin-and-head rest that aided fusion from a viewing distance of 85 cm.

One author and six naïve observers (22-28 years old) with informed consent participated in the study. All observers had normal or corrected-to-normal visual acuity (at least 20/20), clinically acceptable fixation disparity (≦8.6 arc min), central stereopsis (≦40 arc sec), and passed the Keystone vision-screening test.

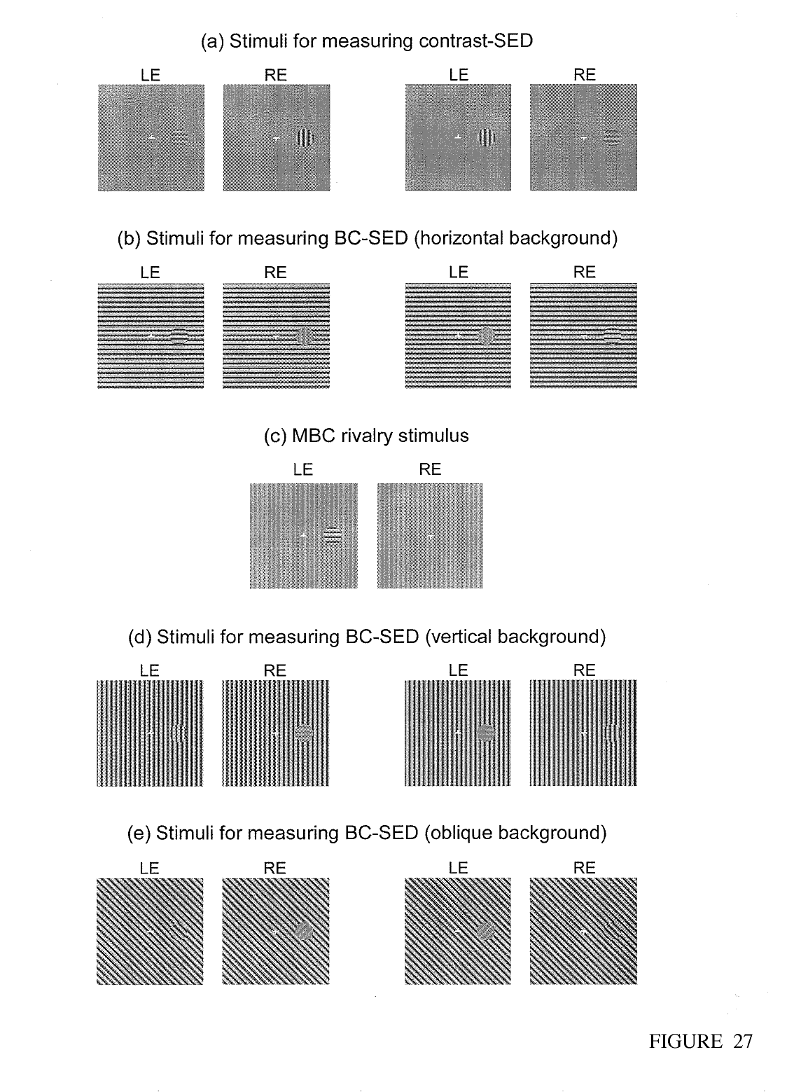

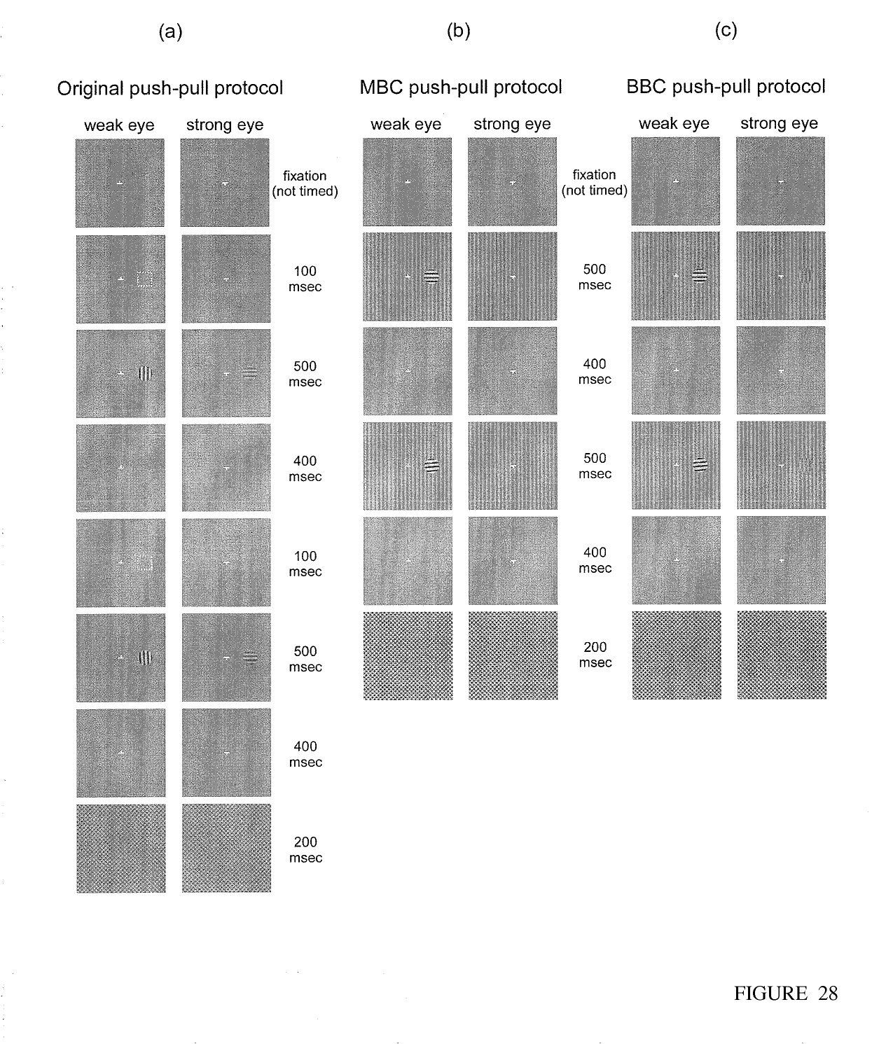

The test stimuli similar to those in FIG. 27b were used to measure the BC-SED at eight concentric retinal locations (0°, 45°, 90°, 135°, 180°, 225°, 270°, and 315°) that are situated 2° in angular distance from the fovea. This allowed the selection two retinal locations with the largest BC-SED (˜40-50° phase-shift), respectively, for training with the MBC push-pull and the BBC push-pull protocols (FIGS. 28b and 28c). The two retinal locations selected were separated from one another by at least 2.83 deg. The two training protocols were implemented, one on each retinal location, on the same day over a 10-day duration (2 sessions/day). The training (learning) effect on BC-SED was monitored before and after each day's training session using stimuli similar to those in FIG. 27b.

Additionally, several other tests were conducted at each training location before and after the training phase to assess the learning effect. These tests were: (1) BC-SED with three different grating stimulus orientations; (2) contrast-SED; (3) stereo threshold.

For measuring BC-SED, the test stimulus comprised a pair of dichoptic vertical (1.2 log unit contrast) and horizontal (1.8 log unit contrast) sinusoidal grating discs (3 cpd, 1.25°, 35 cd/m²), each surrounded by a 7.5°×7.5° horizontal grating background (35 cd/m², 3 cpd, 1.8 log unit contrast) (FIG. 27b). The horizontal grating of the disc had a variable phase-shift (0-180 degrees) relative to the larger horizontal grating background. During the test, a trial began with central fixation on the nonius target (0.45°×0.45°, line width=0.1°, 70 cd/m²) and the presentation of the dichoptic test stimulus (500 msec), followed by a 200 msec mask (7.5°×7.5°, 2-D sinusoidal grating, 3 cpd, 35 cd/m², 1.8 log unit contrast). The observer's task was to judge whether the disc was filled with more horizontal or vertical grating.

A staircase procedure was used to adjust the relative phase-shift of the horizontal grating disc after each trial with a step size of ˜14.2° phase-shift (one pixel), until the observer obtained equal chance of seeing the vertical and horizontal gratings, i.e., the point of equality. Each block of trials (˜50-60 trials) comprised 30 reversals, with the average of the last 26 reversals taken as the final balance phase-shift. When the horizontal grating disc was presented to the LE, its phase-shift at the point of equality is referred to as the LE balance phase-shift (left pair, FIG. 27b). Then the grating half-images were switched between the eyes to obtain the RE balance phase-shift (right pair, FIG. 27b). The difference in the balance phase-shift between the LE and RE is defined as the BC-SED. The measurement at each location was repeated twice.

After the training phase, BC-SED was also measured with horizontal, vertical and oblique background orientation. With the former, the test stimuli were the same as the ones used above (FIG. 27b). However, the method of constant stimuli, instead of the staircase method, was used to obtain the BC-SED. Seven levels of relative phase-shifts of the horizontal grating were tested (0°, 28.4°, 56.8°, 85.3°, 113.7°, 156.3°, 184.7°). Each relative phase-shift level was repeated 7 times/block over 6 blocks. As in the SED test above, the observer responded to seeing either the horizontal or vertical grating (predominant percept). In this, and the remaining SED tests below (including Section 2.5.3), 4 stimulus combinations were tested [2 locations (MBC/BBC)×2 eyes (left/right)]. Each combination was repeated twice. The order of testing was randomized.

For BC-SED measurement with vertical background orientation, the test stimuli are depicted in FIG. 27d, where the vertical (1.8 log unit contrast) and horizontal (1.2 log unit contrast) sinusoidal grating discs (3 cpd, 1.25°, 35 cd/m²) are surrounded by a 7.5°×7.5° vertical grating background (35 cd/m², 3 cpd, 1.8 log unit contrast). The test procedure (staircase) and observer's task were the same as above.

For BC-SED measurement with oblique background orientation, the test stimuli are shown in FIG. 27e. The dichoptic 45° (1.2 log unit contrast) and 135° (1.8 log unit contrast) grating discs (1.25°, 3 cpd, 35 cd/m², 500 msec) are surrounded by a 7.5°×7.5° 135° grating background (3 cpd, 35 cd/m², 1.8 log unit contrast). The test procedure and observer's task were the same as above.

Next, the contrast-SED was measured both before and after the training phase. The stimulus comprised a pair of dichoptic vertical and horizontal sinusoidal grating discs (3 cpd, 1.25°, 35 cd/m²) (FIG. 27a). The contrast of the vertical grating was held constant (1.5 log unit) while the contrast of the horizontal grating was varied (0-1.99 log unit). A trial began with central fixation on the nonius target (0.45°×0.45°, line width=0.1°, 70 cd/m²), the presentation of the dichoptic orthogonal grating discs (500 msec), and terminated with a 200 msec mask (7.5°×7.5° checkerboard sinusoidal grating, 3 cpd, 35 cd/m², 1.5 log unit contrast). The observer responded to his/her percept of seeing more vertical or horizontal grating orientation. The horizontal grating contrast was adjusted after each trial using the QUEST procedure (50 trials/block), until the observer obtained equal chance of seeing the vertical and horizontal gratings, i.e., the point of equality. When the horizontal grating was presented to the LE, its contrast at the point of equality was referred to as the LE balance contrast. The grating discs were switched between the eyes to obtain the RE balance contrast. The difference between the LE and RE balance contrast is defined as the contrast-SED.

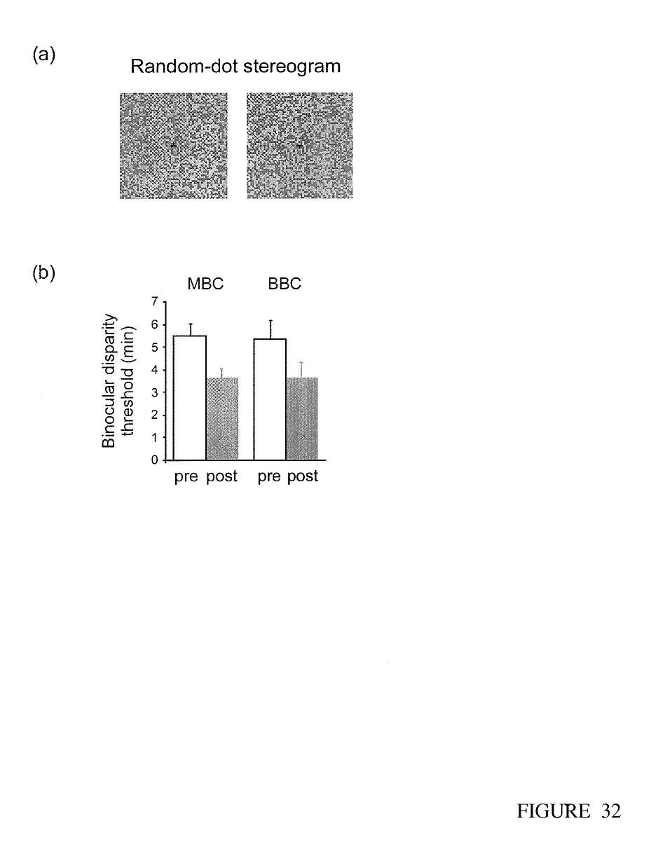

For the stereo threshold test, a 7.5°×7.5° random-dot stereogram (dot size=0.0132°, 35 cd/m²) with a variable crossed-disparity disc target (1.25°) was used (FIG. 32a). The contrast of the stereogram was individually selected for each observer, to make the stereo task moderately difficult and to avoid a possible ceiling-effect due to pixel-size constraint. With this criterion, the contrast levels were variously set for different observers (1.2 log unit: 1 observer, 1.3 log unit: 3 observers, 1.5 log unit: 1 observer; 1.7 log unit: 2 observers).

The standard 2AFC method was used in combination with the staircase procedure to measure stereo disparity threshold. The temporal sequence of stimulus presentation was: fixation, interval-1 (200 msec), blank (400 msec), interval-2 (200 msec), blank (400 msec), and random-dot mask (200 msec, 7.5°×7.5°, 35 cd/m²). The observer indicated whether the crossed-disparity disc was perceived in interval-1 or -2, and an audio feedback was given. Each block comprised 10 reversals (step size=0.8 arc min, total ˜50-60 trials), with the last 8 reversals taken as the average threshold. Each block was repeated 4 times, and measured over two days.

B. Push-Pull and BBC Push-Pull Protocols

The two retinal locations chosen for training were randomly assigned to the two protocols, which are the same in all aspects except for the design of the binocular rivalry stimuli employed (FIGS. 28b & 28c). The stimulus design for the MBC push-pull protocol had a horizontal sinusoidal grating disc (3 cpd, 1.25°, 35 cd/m², 1.8 log unit contrast) that was surrounded by a 7.5°×7.5° vertical grating background (3 cpd, 35 cd/m², 1.2 log unit contrast) in one half-image. The other half-image had a homogeneous vertical grating (3 cpd, 35 cd/m², 7.5°×7.5°, 1.2 log unit contrast). The binocular rivalry stimulus design in the BBC push-pull protocol also had one half-image with the horizontal sinusoidal grating disc and vertical background. However, the other half-image had a vertical sinusoidal grating disc at the location corresponding to the horizontal grating disc in the first half-image. The vertical grating disc was created by phase-shifting a circular region of the vertical grating surrounding it by 180°. All other parameters of the BBC stimulus design were the same as those for the MBC stimulus design.

The stimulation sequence for both protocols was identical. During the training, a trial began with fixation at the nonius target. Then, at the training location, the MBC or BBC stimulus was presented for 500 msec, and 400 msec later, a second MBC or BBC stimulus was presented for another 500 msec (FIGS. 28b and 28c). The horizontal grating of the disc in the second presentation had a slightly different orientation from the horizontal grating in the first presentation. Four hundred msec after the second presentation, the onset of a binocular checkerboard sinusoidal grating mask terminated the trial (200 msec, 7.5°×7.5°, 3 cpd, 35 cd/m², 1.8 log unit contrast). Notably, due to its higher contrast and stronger boundary contour, the horizontal grating disc in the weak eye was always perceived during each presentation interval because it successfully suppressed the corresponding vertical grating in the strong eye. The observer reported whether the first or second horizontal grating disc had a slight counterclockwise orientation, and an audio feedback was given. Fifty such training trials were run for each training block. The QUEST procedure was employed to obtain the orientation discrimination threshold at the end of the 50-trial block. Altogether, twelve blocks were performed during each training session/day, for each training protocol.

The two protocols were implemented on each training day, in an interleaved manner. Training on each protocol lasted for an hour, which was performed either in the morning or afternoon session.

C. Results

1. Learning Effect on BC-SED During the Training Phase

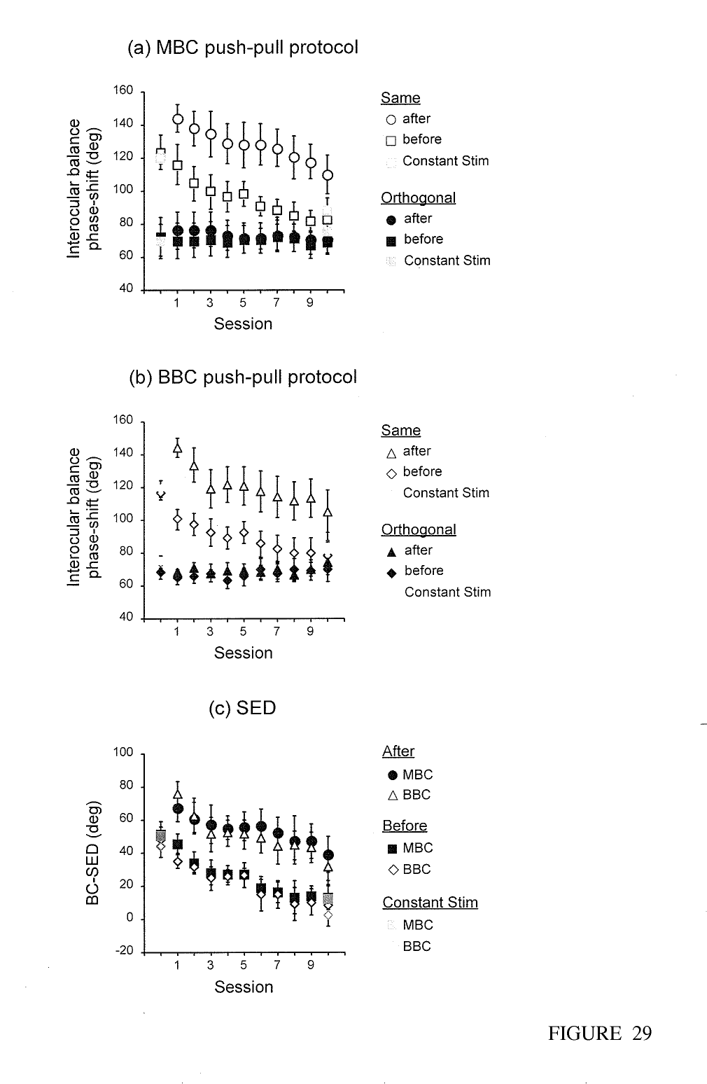

The average interocular balance phase-shift data obtained during the training-phase are shown in FIGS. 29a and 29b, respectively, for the MBC push-pull and BBC push-pull protocols. The open symbols represent the results from the test stimuli where the test grating orientation (in the disc) was the same as the trained grating orientation (in the disc). For simplicity, these results are referred to as the “same” data. The closed symbols represent the results with test stimuli that were orthogonal to the trained orientation (in the disc). These results are referred to as the “orthogonal” data. [Note that the test (FIG. 27b) and training (FIGS. 28b and 28c) stimuli had different background grating orientation.]

At the MBC training location (FIG. 29a), the same interocular balance phase-shift decreases as the training progressed when it was measured before (open black squares, slope=−3.915, R²=0.917, p<0.001) and after (open black circles, slope=−3.188, R²=0.943, p<0.001) each day's training session. On the other hand, there is little change in the orthogonal interocular balance phase-shift measured before (filled black squares, slope=−0.153, R²=0.136, p=0.265) and after (filled black circles, slope=−0.697, R²=0.752, p=0.001) each day's training session. Two-way ANOVA with repeated measures confirm that the slope in the orthogonal data is significantly shallower than that in the same data [interaction effect between testing stimuli (same/orthogonal) and training session: before, F(10, 60)=10.903, p<0.001; after, F(9, 54)=2.098, p=0.046]. This demonstrates the orientation specificity of the perceptual learning effect. It is also clear that there is a significant difference in the interocular balance phase-shift measured before and after each day's training session for the same data (main effect of the before-after: F(1,6)=91.176, p<0.001; interaction effect between the before-after and training session: F(9, 54)=0.653, p=0.747; 2-way ANOVA with repeated measures). However, there is no significant difference in the before and after interocular balance phase-shift for the orthogonal data [main effect of the before-after: F(1,6)=3.227, p=0.123; interaction effect between the before-after and training session: F(9, 54)=0.638, p=0.760; 2-way ANOVA with repeated measure].

The interocular balance phase-shift data at the BBC-training location (FIG. 29b) show a similar trend. There is a clear learning effect in the same data [before: open black diamonds, slope=−3.193, R²=0.863, p<0.001; after: open black triangles, slope=−3.382, R²=0.817, p<0.001] but no reliable learning effect in the orthogonal data [before: filled black diamonds, slope=0.410, R²=0.357, p=0.052; after: filled black triangles, slope=0.250, R²=0.149, p=0.271]. A significant before-after difference in interocular balance phase-shift is only observed in the same data [main effect of the before-after: F(1,6)=65.113, p<0.001; interaction effect between the before-after and training session: F(9, 54)=1.431, p=0.198; 2-way ANOVA with repeated measure], and not in the orthogonal data [main effect of the before-after: F(1,6)=0.613, p=0.463; interaction effect between the before-after and training session: F(9, 54)=1.024, p=0.434; 2-way ANOVA with repeated measure].

FIG. 29c plots the average BC-SED, which is defined as the difference between the same and orthogonal interocular balance phase-shift values of the corresponding conditions. BC-SED is significantly reduced, i.e., showing a learning effect, for all four sets of data [before/MBC: slope=−3.762, R²=0.898, p<0.001; after/MBC: slope=−2.490, R²=0.899, p<0.001; before/BBC: slope=−3.603, R²=0.911, p<0.001; after/BBC: slope=−3.631, R²=0.835, p<0.001]. The learning effect on BC-SED is similar at the two training locations with the MBC and BBC protocols, no matter whether they were measured before each day's training session [main effect of training protocol (MBC/BBC): F(10,60)=11.792, p<0.001; interaction effect between the training protocol and training session: F(10,60)=1.611, p=0.125; 2-way ANOVA with repeated measures], or after the training session [main effect of training protocol (MBC/BBC): F(9,54)=4.562, p<0.001; interaction effect between the training protocol and training session: F(9,54)=0.481, p=0.881; 2-way ANOVA with repeated measures].

Altogether, the above observations during training with both MBC and BBC push-pull protocols demonstrate that learning to reduce BC-SED is possible without resorting to explicit attention cueing during the training. This finding reinforces the notion that suppression of the stimulus presented to the strong eye is the important factor triggering a significant plasticity within the interocular inhibitory network.

2. Assessing the Learning Effect on Specific Visual Functions Tested Before and After the Training

a. Learning Effect on BC-SED with Horizontal Grating Background

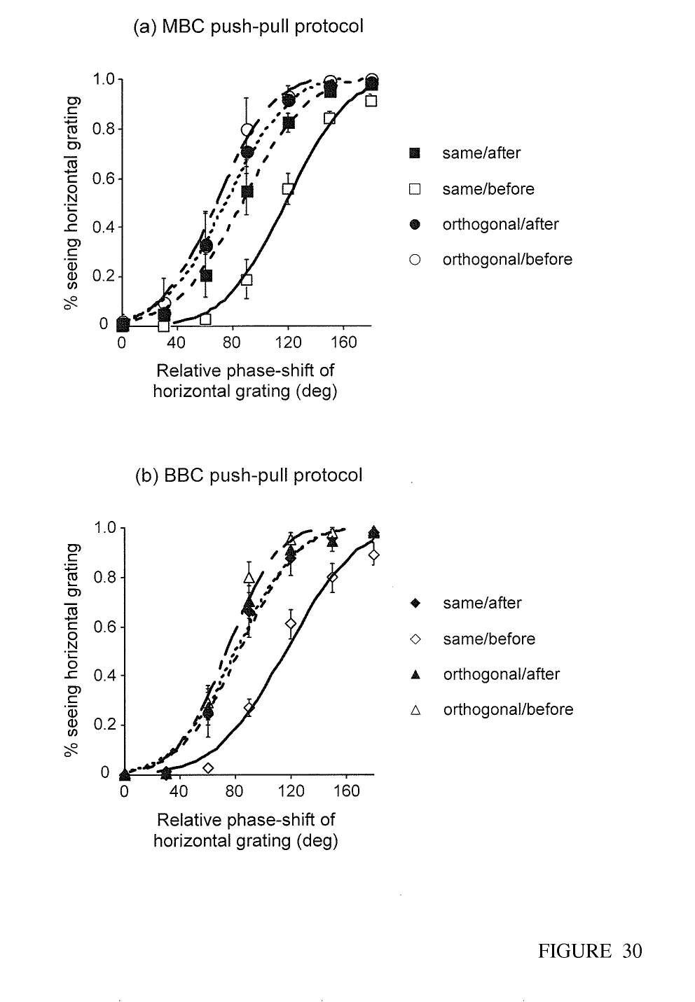

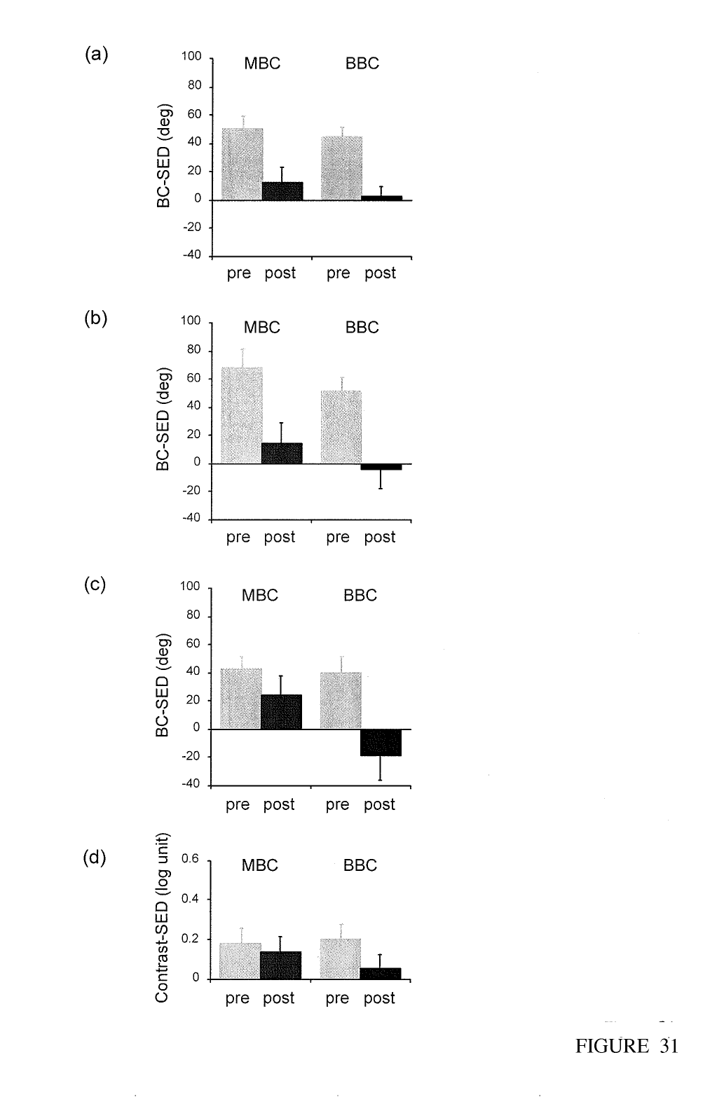

Besides using the staircase method, the method of constant stimuli was used to measure the weak and strong eyes' interocular balance phase-shift with the test stimuli in FIG. 27b FIGS. 30a and 30b, respectively, plot the average data for the MBC and BBC push-pull training locations with fitted psychometric functions (cumulative normal distribution functions). Separately, probit analysis was applied to each observer's data to estimate the interocular balance phase-shift (phase-shift at 50% performance level) and the standard deviation of the normal distribution of each psychometric function. The average interocular balance phase-shift data are plotted with gray symbols in FIG. 29 (pre-training at day 0 and post-training at day 10) and they show a similar trend as the ones measured with the staircase method during the training phase (section 3.1). At the MBC push-pull training location, there is a signification decrease in interocular balance phase-shift in the weak eye [same data: pre: 120.4±4.0 deg, post: 88.5±7.0 deg, t(6)=4.753, p=0.003], but little change in the strong eye [orthogonal data: pre: 69.7±10.5 deg, post: 75.736±8.557 deg, t(6)=−1.309, p=0.238]. A similar trend is found at the BBC push-pull training location [same interocular balance phase-shift: pre: 119.1±5.2 deg, post: 83.8±8.6 deg, t(6)=5.477, p=0.002; orthogonal interocular balance phase-shift: pre: 74.8±3.9 deg, post: 80.9±6.7 deg, t(6)=−0.967, p=0.371]. The pre- and post-BC-SED data are plotted in a bar graph in FIG. 31a. BC-SED is significantly reduced at both the MBC [pre: 50.7±8.5 deg, post: 12.8±10.9 deg, t(6)=3.887, p=0.008] and BBC push-pull [pre: 44.3±6.7 deg, post: 2.8±7.2 deg, t(6)=5.086, p=0.002] training locations. Although the BC-SED reduction at the MBC training location (37.9 deg) is smaller than that at the BBC training location (41.5 deg), their difference fails to reach the significant level [Main effect of training condition: F(1,6)=1.592, p=0.254; main effect of training session: F(1,6)=22.051, p=0.003; interaction effect between training condition and session: F(1,6)=0.326, p=0.589, 2-way ANOVA with repeated measures].

b. Learning Effect on BC-SED with Vertical Grating Background

FIG. 31b illustrates the average BC-SED measured using the test stimuli with vertical grating background (FIG. 27d) in the pre and post training phase. Clearly, the BC-SED data are reduced at both the MBC [t(6)=3.360, p=0.015] and BBC push-pull [t(6)=4.420, p=0.004] training locations. Although the BC-SED reduction at the MBC training location is smaller than that at the BBC training location, statistical analysis fails to reveal a significant difference between the two [Main effect of training session: F(1,6)=17.980, p=0.005; interaction effect between training location and session: F(1,6)=0.045, p=0.840, 2-way ANOVA with repeated measures].

c. Learning Effect on BC-SED with Oblique Grating Background

FIG. 31c depicts the average BC-SED data measured with the test stimuli with oblique grating background (FIG. 27e) before and after the training. The reduction in BC-SED is significantly smaller at the MBC training location than at the BBC training location [Main effect of training session: F(1,6)=10.317, p=0.018; interaction effect between training location and session: F(1,6)=22.237, p=0.003, 2-way ANOVA with repeated measures]. Further analysis reveals a significant reduction at the BBC training location [t(6)=4.111, p=0.006] but not at the MBC training location [t(6)=1.652, p=0.150].

Overall, both the MBC and BBC push-pull training protocols were found to effectively reduce the BC-SED when the test stimuli (vertical/horizontal grating discs) are similar to the training stimuli (FIGS. 30, 31a and 31b). The BBC push-pull protocol was also found to significantly reduce BC-SED when the training (vertical/horizontal) and test (oblique) stimuli have different orientations (FIGS. 27e & 31c). This finding, that the learning to reduce BC-SED is transferable to a test stimulus whose orientation is 45° from the trained orientation, resembles the earlier finding using the push-pull protocol with attention cueing. The learning is attributed largely to the plasticity of interocular inhibition on the boundary contour mechanism. It should be emphasized that this conclusion could be reached because the oblique test stimuli (FIG. 27e) revealed the operation of the surface BC mechanism more substantially than that of the surface feature mechanism. On the other hand, while test stimuli such as those in FIG. 27a may reveal a BC-SED learning effect, they do not allow differentiation of the relative contributions of the surface boundary contour from the surface feature mechanisms.

More generally, a significance is attached to the finding that learning transfers to test stimuli (FIG. 27e) that are oriented 45° from the training stimuli (FIG. 31c). This is in contrast to the earlier finding that revealed a much reduced learning when the test stimuli were oriented 45° away from the training stimuli. Together, they argue for the notion that the surface BC mechanism has broader orientation tuning functions than the surface feature mechanism.

Also notable, is that the learning effect on BC-SED is larger at the BBC push-pull training location than at the MBC push-pull training location. In particular, the learning effect on the BC-SED with oblique grating background shown in FIG. 31c is significantly larger at the BBC than at the MBC training location. This tendency suggests that the suppression of the BC in the half-image presented to the strong eye during training more effectively reduces SED. The MBC push-pull protocol is not as effective because the half-image presented to the strong eye does not have a BC (i.e., there is no BC suppression but only surface-feature suppression in the strong eye).

3. Learning Effect on Contrast-SED

FIG. 31d depicts the average contrast-SED measured before and after the training phase with the test stimuli shown in FIG. 27a. The reduction in contrast-SED is significantly larger at the BBC than the MBC push-pull training location [Main effect of training session: F(1,6)=5.274, p=0.061; Interaction effect between training condition and session: F(1,6)=10.328, p=0.018, 2-way ANOVA with repeated measures]. Further analysis reveals that the reduction is significant at the BBC training location [t(6)=3.625, p=0.011] but not at the MBC training location [t(6)=0.758, p=0.477]. This finding is consistent with the observations of the BC-SED with oblique grating background in FIG. 31c, where the BBC push-pull training results in a significantly stronger learning effect than the MBC push-pull training. Both findings together support the notion that binocular suppression of boundary contour in the strong eye during the training more effectively reduces sensory eye dominance.

4. Learning Effect on Stereo Threshold

FIG. 32b plots the average stereo thresholds measured with random dot stereograms (FIG. 32a) at the MBC and BBC push-pull training locations. A similar learning effect is found at both training locations [Main effect of the training session: F(1,6)=98.025, p<0.001; Interaction effect between training location and session: F(1,6)=1.655, p=0.246, 2-way ANOVA with repeated measures]. Further analysis reveals significant decreases of binocular disparity thresholds at both training locations [MBC: t(6)=9.191, p<0.001; BBC: t(6)=9.421, p<0.001]. This learning effect on binocular depth perception, a visual function that was not trained, is similar to those found in previous studies that used the push-pull protocol with attention cueing.

### Example 5

### A Binocular Perimetry Study of the Causes and Implications of Sensory Eye Dominance

A. Experimental Procedures

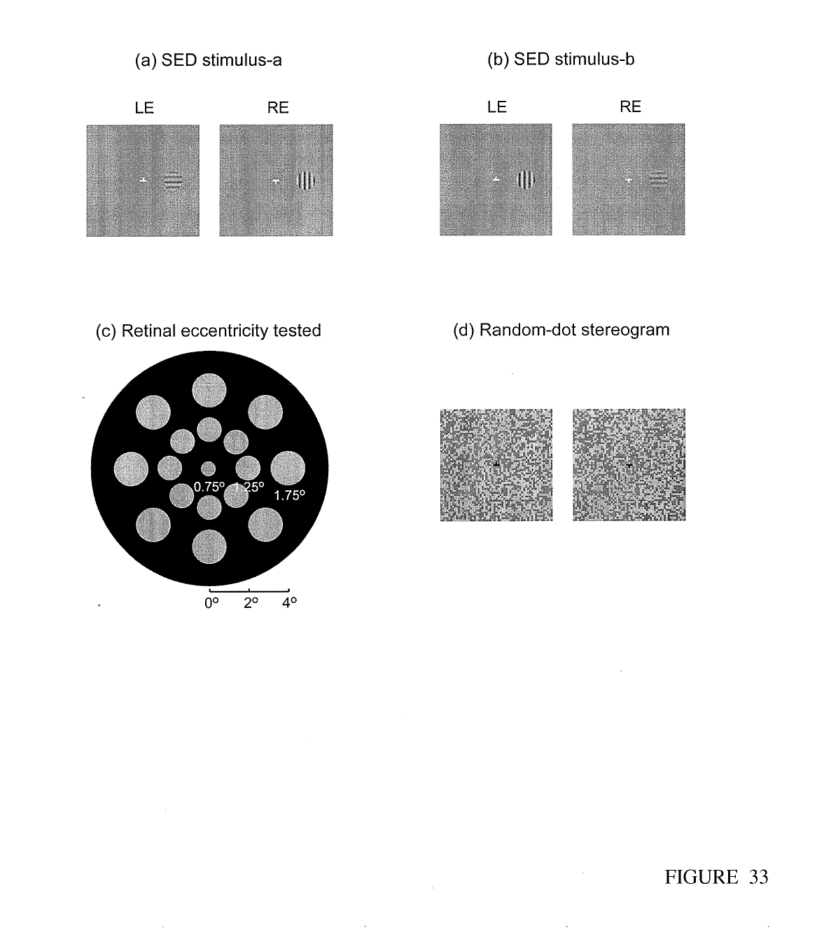

The stimuli were generated using either a Macintosh G4 or MacPro computer running Matlab and Psychophysics Toolbox, and presented on a 19-inch flat CRT monitor. The resolution of the monitor was set at 1280×1024 @ 100 Hz refresh rate for all experiments, except for the stereopsis experiment where the resolution was 2048×1536 @ 75 Hz. All observers (one author and eleven naïve observers with informed consent) had self-reported normal binocular vision. Each observer's performance was measured in the following order: local SED, interocular difference in contrast threshold, stereo disparity threshold and stereo reaction time at 17 retinal locations (FIG. 33c). The 17 retinal locations were the fovea and eight concentric locations (0°, 45°, 90°, 135°, 180°, 225°, 270°, and 315°), 2° and 4°, respectively, from the fovea. Additionally, the observers' motor eye dominance (MED) and binocular rivalry performance was measured with central viewing. For the binocular rivalry experiment, only ten out of the twelve observers were tested, as two observers were unavailable for the test.

All twelve adult observers (ages 21-29) had normal or corrected-to-normal visual acuity (at least 20/20), clinically acceptable fixation disparity (≦8.6 arc min) and stereopsis (≦40 arc sec). They also passed the Keystone vision-screening test. During the experiments they viewed the computer monitor through a haploscopic mirror system attached to a head-and-chin rest from a distance of 85 cm.

For the interocular imbalance test to measure SED, the stimulus comprised a pair of dichoptic vertical and horizontal sinusoidal grating discs (35 cd/m²) on a gray background (11°×11°, 35 cd/m²) (FIG. 33a). The contrast of the vertical grating was held constant (1.5 log unit) while the contrast of the horizontal grating was variable (0-1.99 log unit). For testing at the 2° or 4° eccentric retinal location, a trial began with central fixation on the nonius target (0.45°×0.45°, line width=0.1°, 70 cd/m²) and the presentation of the dichoptic orthogonal gratings (500 msec), followed by a 200 msec mask (11°×11° checkerboard sinusoidal grating, 35 cd/m², 1.5 log unit). It should be noted that the stimulus duration of 500 msec is sufficiently long to activate the underlying interocular inhibitory mechanism. The testing at the foveal location was similar, except the nonius fixation was removed 200 msec before the presentation of the stimulus. The observer responded to his/her percept by key presses. If a mixture of vertical and horizontal orientation was seen, the observer would respond to the predominant orientation perceived. The horizontal grating contrast was adjusted after each trial with the QUEST procedure and ended after 50 trials (block). When the horizontal grating was presented to the LE (FIG. 33a) its contrast at equal predominance is referred to as the LE's balance contrast. To obtain the RE's balance contrast, the gratings were switched between the eyes (FIG. 33b). The difference between the LE and RE balance contrast values is defined as the local SED for that tested retinal location. In all, the 17 retinal locations were tested with 34 stimulus combinations (17 locations×2 eyes/orientation). The order of the retinal location tested was randomized and each location was tested twice.

Because the SED was measured at different retinal eccentricities (0°, 2° and 4°), the cortical magnification factor was applied to the stimulus parameters used for testing. The grating disc stimuli for the foveal location was fixed at 5 cpd and 0.75° (disc diameter). For the eccentric stimulation, the stimuli's spatial frequency and disc diameter were proportionally scaled using the cortical magnification factor given by the formula: target frequency (cpd)=foveal frequency/[1+eccentricity (°)/3]; target size (°)=foveal size*[1+eccentricity (°)/3]. Accordingly, [3 cpd, 1.25°] was used for the grating at 2° eccentricity, and [2.14 cpd, 1.75°] was used for the grating at 4° eccentricity. The spatial frequency used for the checkerboard mask was consistent with that of the grating disc.

The Monocular contrast detection threshold was also tested. The tested eye viewed a monocular sinusoidal grating (35 cd/m², 500 msec) that was oriented either horizontal or vertical. The fellow eye viewed a homogeneous field. The contrast sensitivity test was conducted using a 2AFC method in combination with the QUEST procedure. The 2AFC stimulus presentation sequence was: fixation, interval-1 (500 msec), blank (400 msec), interval-2 (500 msec), blank (400 msec) and mask (11°×11° checkerboard sinusoidal grating, 35 cd/m², 1.5 log unit contrast, 200 msec). The monocular grating was presented at one interval while the other interval had a blank field. For testing at the fovea, the nonius fixation was removed 200 msec before the presentation of the stimulus. The observer responded by key press whether he/she saw the grating in interval-1 or -2, and an audio feedback was given. The grating contrast was adjusted after each trial (by QUEST) to obtain the contrast threshold.

The monocular contrast threshold was measured at the same 17 retinal locations used to measure SED. The cortical magnification factor was appropriately accounted for by scaling the grating spatial frequency and disc diameter at each retinal eccentricity (fovea: 5 cpd, 0.75°; 2°: 3 cpd, 1.25°; 4°: 2.14 cpd, 1.75°). A total of 68 stimulus combinations (17 locations×2 eyes×2 orientations) were tested in a randomized order. Each stimulus combination was repeated over 2 blocks of trials (50 trials/block).

For stereo threshold and reaction time, an 11°×11° random-dot stereogram (dot size=0.0132°, 35 cd/m², 1.5 log unit contrast) with a variable crossed-disparity disc target was used (disc diameters: 0.75° at fovea; 1.25° at eccentricity 2°; 1.75° at eccentricity 4°) (FIG. 33d). The standard 2AFC method in combination with the staircase procedure was employed to measure the stereo disparity threshold. The temporal sequence of the stimulus presentation was fixation, interval-1 (200 msec), blank (400 msec), interval-2 (200 msec), blank (400 msec), and random-dot mask (200 msec, 11°×11°, 35 cd/m²). For testing at the fovea, the nonius fixation was removed 200 msec before the presentation of the stimulus. The observer indicated whether the crossed-disparity disc (front depth) was perceived in interval-1 or -2, and an audio feedback was given. Each block comprised 10 reversals (step size=0.8 arc min, total ˜50-60 trials), and the average of the last 8 reversals were taken as the stereo threshold. Stereo threshold was measured at each of the same 17 retinal locations in a randomized testing order. Each block was repeated twice.

The binocular disparity of the dichoptic disc target used to measure stereo reaction time (RT) was either ±6 arc min. The disc diameter was appropriately adjusted for cortical magnification as in the above. The observer began a trial by aligning his/her eyes on the nonius fixation. The target was then presented at one of the seventeen retinal locations (the nonius fixation would be removed 200 msec before the stimulus presentation if the fovea was tested). The observer's task was to press one of two keys immediately upon detecting the stereo disc to indicate whether it was in front or back. Upon his/her response, the stimulus was removed and a blank screen (400 msec) was presented. This was followed by a mask (200 msec) that ended the trial, after which an audio feedback was given. If depth (target) was not detected, the stimulus timed-out after 2500 msec. Each test block consisted of 60 trials, with 30 front-trials and 30 back-trials that were randomly interleaved. Three blocks were tested at each of the 17 retinal locations.

To control for the accuracy of response in the RT task, each observer was given several practice blocks until he/she achieved an accuracy of 70% or higher (accuracy is defined as the ratio of correct trials to the total number of trials). Moreover, only correct trials whose response times were longer than 100 msec were used in the final data analysis. Fewer than 0.05% correct trials had an RT of <100 msec. The observers' average accuracy was also found to be quite high (90%) during the test sessions.

For Binocular rivalry tracking, the stimulus comprised a pair of dichoptic vertical and horizontal grating discs (1°, 5 cpd, 35 cd/m², 1.99 log unit contrast) surrounded by a 7.5°×7.5° gray square (35 cd/m²). The observer aligned his/her eyes on the nonius fixation (0.45°×0.45°, line width=0.1°, 70 cd/m²) to prepare for a trial. He/she then pressed the spacebar to remove the nonius fixation. This was followed 200 msec later, by the presentation of the binocular rivalry stimulus (30 sec). A 1-sec mask (7.5°×7.5° checkerboard sinusoidal grating, 3 cpd, 35 cd/m², 1.99 log unit contrast) terminated the trial. The observer's task was to report (track) his/her instantaneous percept of the binocular rivalry stimulus over the 30 sec stimulus viewing duration. Depending on the percept, vertical, horizontal, or a mixture of both, he/she would depress the appropriate key until the next percept took over. A total of 8 trials were performed (2 orientation/eyes×4 repeats).

For motor eye dominance, a variation of the Ring sighting test was used. To perform the test, the observer brought both hands simultaneously to the front of his/her face at arms length, and formed a ring (2-3 inches in diameter) by bringing together the index finger and thumb from each hand. He/she then sighted a target with both eyes opened through this “ring”, while carefully placing the sighted target in the center of the ring. After this, he/she closed each eye alternately to determine whether the right or left eye saw the target as more centered in the ring. The eye that saw the target as more centered is defined as the motor-dominant eye.

B. Results

1. Perimetry Results of Individual Observers (N=12)

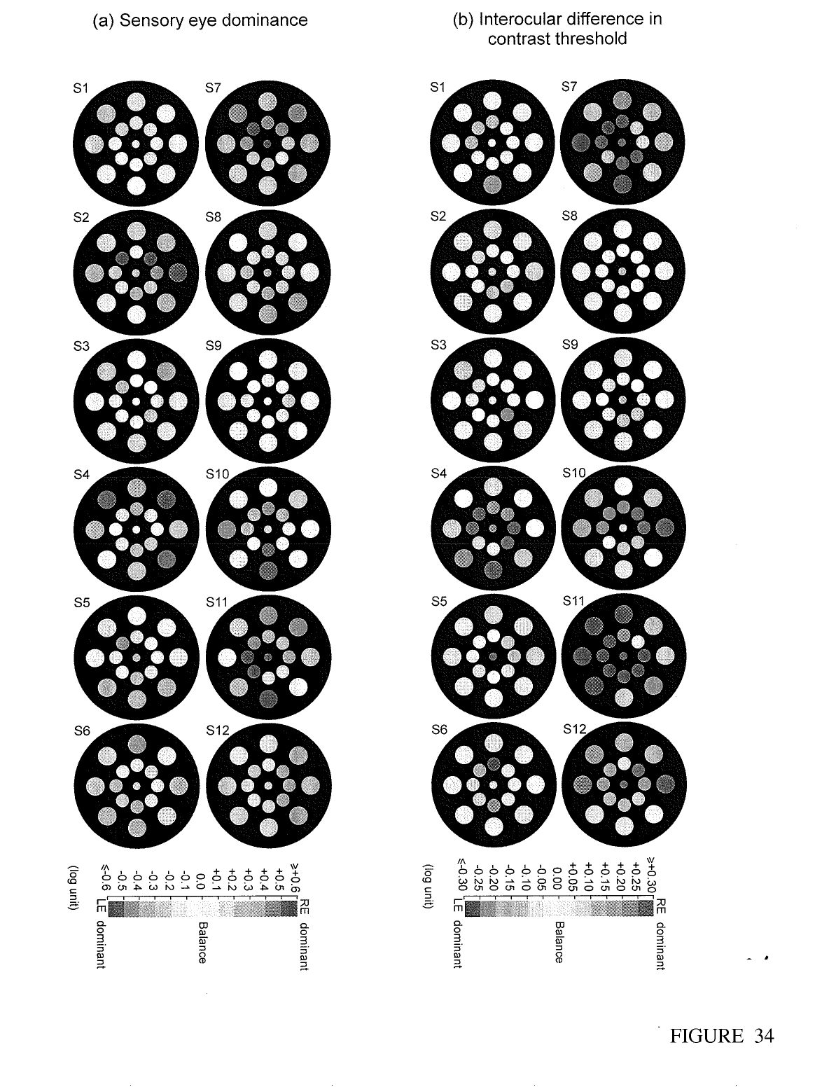

FIGS. 34a and 34b, respectively, plot each observer's sensory eye dominance (SED) and interocular difference in contrast threshold at the 17 tested locations. The data are represented with a green-yellow-red color spectrum that corresponds to the extent and sign of SED or interocular difference in contrast threshold (red indicates RE being stronger while green indicates LE being stronger). Clearly, most plots have a varying color spectrum rather than a single color. This indicates that SED and the interocular difference in contrast threshold vary across the visual field both in extent and sign. Furthermore, a careful comparison between the SED and interocular difference in contrast threshold measurements at the same test location reveals that they do not always indicate the same eye to be superior. Consider, for example, the results of observer 12 (S12) at the leftmost test location. S12's SED measurement indicates left eye superiority (green) whereas the interocular difference in contrast threshold measurement reveals right eye superiority (red). This finding supports that a difference in monocular contrast detection between the two eyes is not always correlated with SED.

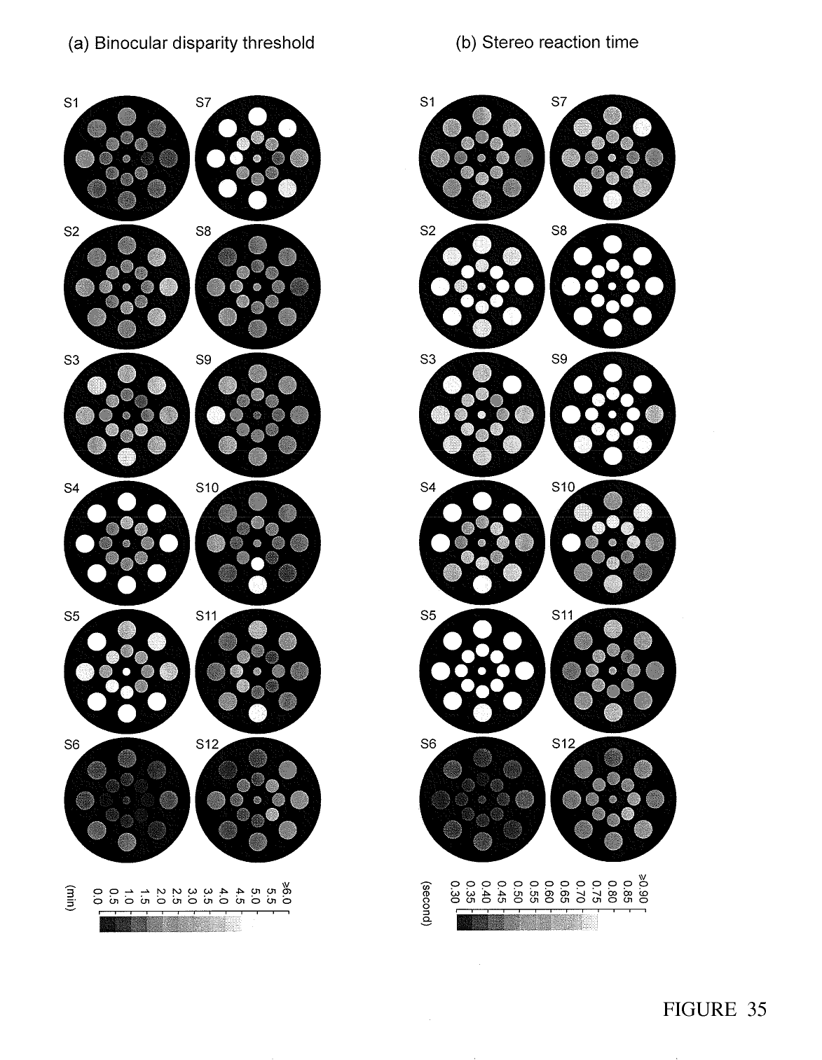

FIGS. 35a and 35b, respectively, plot each observer's binocular disparity threshold and reaction time to detect binocular depth. These data are plotted with a dark-light gray shade that represents the different gradients of the data (a smaller measured value has a darker gray shade). As in the SED and interocular difference in contrast threshold data in FIG. 34, the general trend of the depth perception data exhibits an inhomogeneity across the visual field. To further understand the implications of these results and their relationships, a series of further data analyses were performed.

2. Analysis of Visual Field Eccentricity and Symmetry

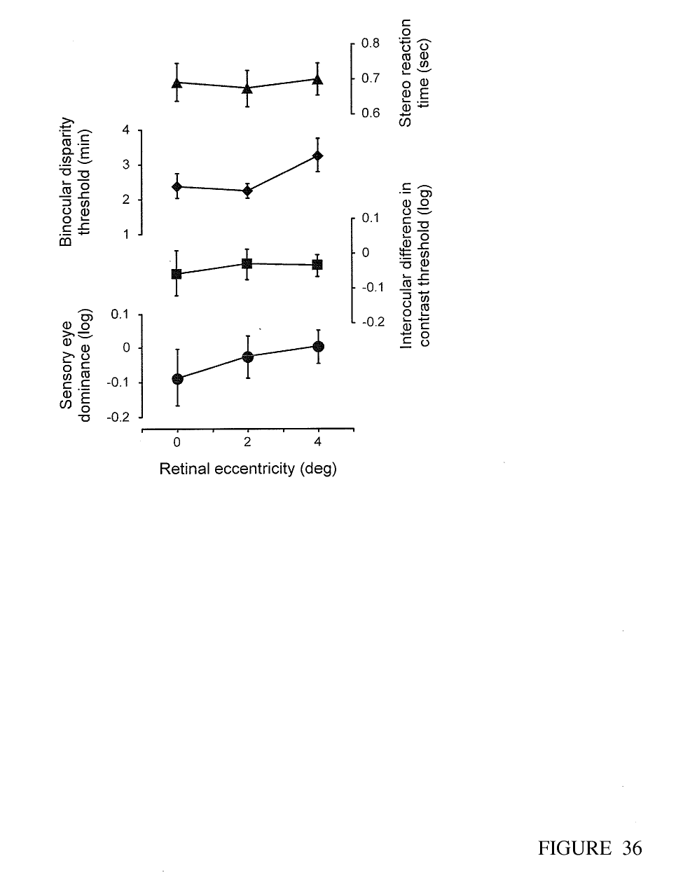

First the data was averaged from the same retinal eccentricity for each of the measured function (SED, interocular difference in contrast threshold, disparity threshold, and stereo reaction time). These are plotted in FIG. 36 as a function of the tested retinal eccentricity (fovea: 0; parafovea: 2°, 4°). A one-way ANOVA was then applied with repeated measures to each set of test results. It was found that binocular disparity threshold (diamonds) significantly increases with retinal eccentricity [F(2, 22)=4.187, p=0.029]. On the other hand, the remaining three measurements do not reliably change with eccentricity (p>0.15) (SED: circles; interocular difference in contrast threshold: squares; reaction time to detect depth: triangles).

Also examined was whether there was a performance asymmetry between the upper and lower visual field, or left and right visual field. Paired t-test analysis reveals that the reaction time to detect binocular depth in the right visual field is 23 msec faster than in the left visual field. However, this difference is not significant after applying the pairwise t-test with the Bonferroni correction [t(11)=2.435, p=0.033, which is larger than acceptable p=0.05/2=0.025]. Other measurements also do not show any significant asymmetric effect (p>0.105).

3. Gradual Spatial Variation of SED and Interocular Difference in Contrast Threshold

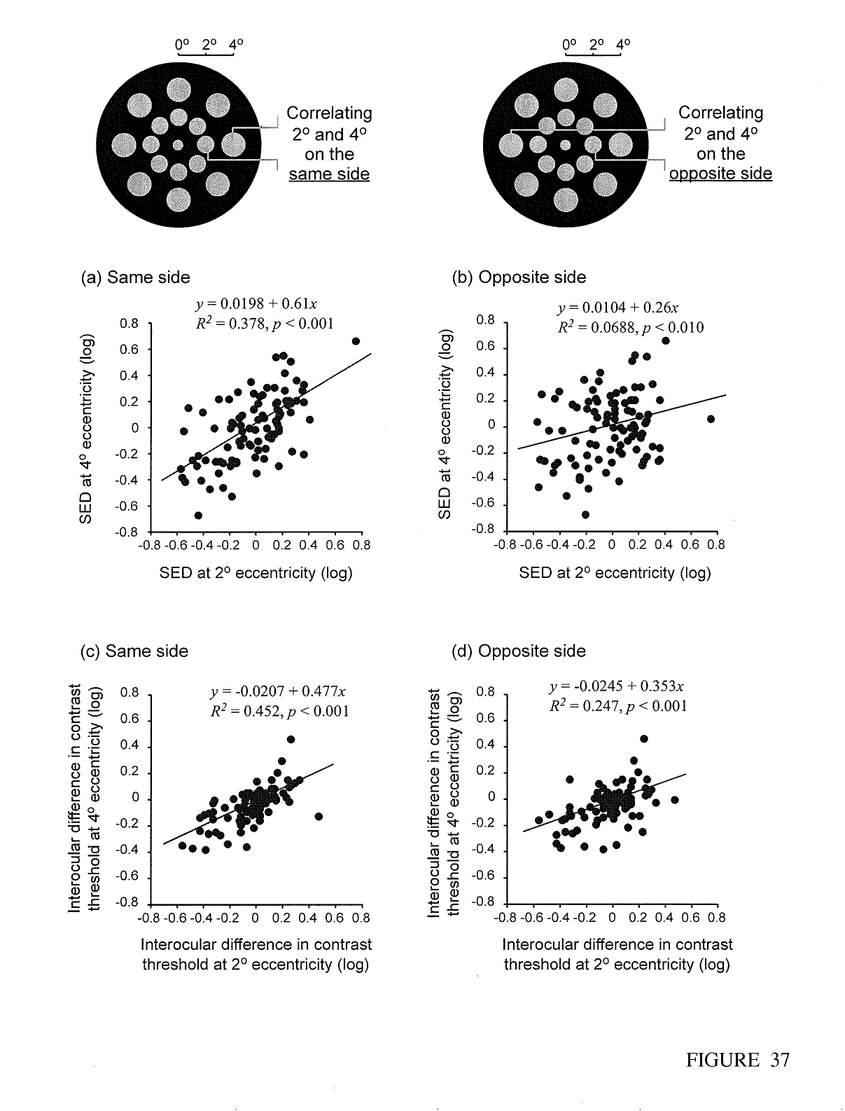

As plotted in FIG. 34, individual measures of SED and interocular difference in contrast threshold vary across the visual field (inhomogeneity). Yet, an inspection of the plots shows that the variations between adjacent locations are more gradual than abrupt. To quantify this impression of a gradual change, the correlation between adjacent SEDs was examined on the same side of the retina along the same radial direction. This is done by correlating adjacent SEDs at the 2° and 4° eccentricity, as shown in FIG. 37a. Since there are 8 pairs of data points for each observer, and 12 observers were tested, FIG. 37a has a total of 96 data points. Secondly, the SED at 2° was correlated with the SED at 4° across the fovea (i.e., along the opposite radial direction) and plotted in FIG. 37b. Consistent with the notion of a gradual change, a larger correlation was found in FIG. 37a (adjacent location) (R²=0.378, p<0.001) than in FIG. 37b (across fovea) (R²=0.0688, p=0.010).

A similar analysis was applied to the interocular difference in contrast threshold data and also yielded a stronger correlation between the 2° and 4° data points when they are adjacent on the same side of the retina (FIG. 37c, R²=0.452, p<0.001) than when they are across the fovea (FIG. 37d, R²=0.247, p<0.001).

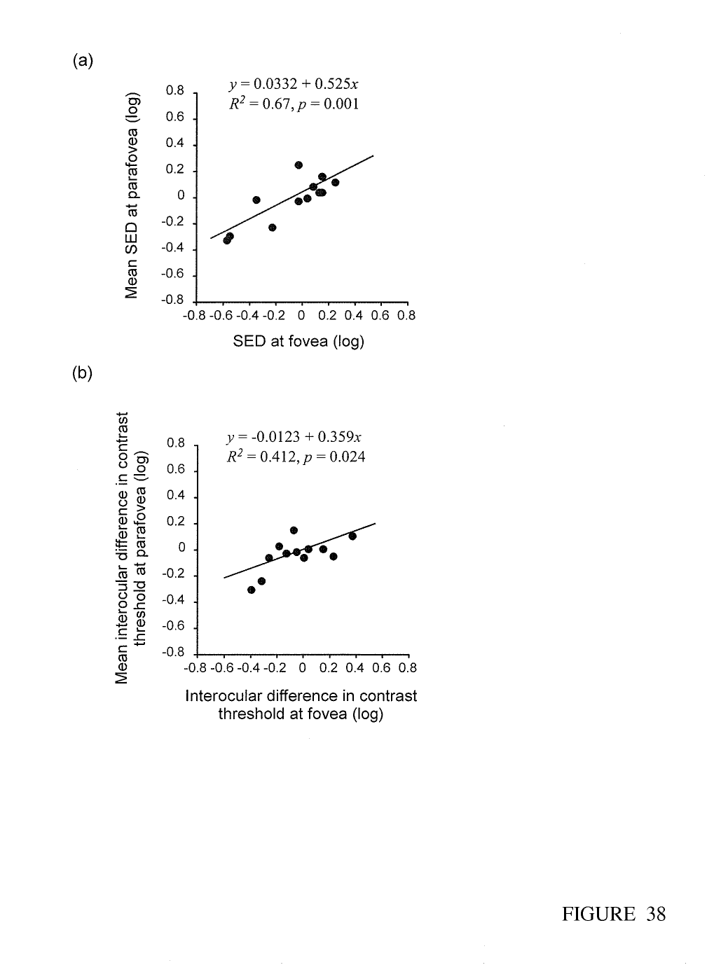

In addition, the correlation between the fovea and the parafoveal regions were examined (average of all 2° and 4° data). Each data point in FIG. 38a represents the results of one observer, where the x-value is foveal SED and y-value is the mean of sixteen SEDs from the 2° and 4° test locations. A reliable correlation was found between the foveal and parafoveal SED (R²=0.670, p=0.001). The data of the interocular difference in contrast threshold was plotted in a similar manner (FIG. 38b) and found a similar trend (R²=0.412, p=0.024). Altogether, the data in FIG. 38 suggest that for both the SED and interocular difference in contrast threshold, a sample measurement at the fovea can provide a reasonably good prediction of the global trend in the parafoveal region.

Finally, the visual field variation of stereopsis was examined using similar analyses. For both binocular disparity detection threshold and reaction time, there are significant correlations between the fovea and parafoveal performance (binocular disparity: R²=0.384, p=0.031; reaction time: R²=0.908, p<0.001). In the parafoveal region, there is a stronger correlation between the 2° and 4° disparity threshold data when they are adjacent on the same side of the retina (R²=0.255, p<0.001), than when they are across the fovea (R²=0.102, p<0.001). However, this trend is not found for the stereo reaction time data (adjacent: R²=0.646, p<0.001; across: R²=0.642, p<0.001).

4. SED Cannot be Fully Accounted for by a Difference in Interocular Contrast Threshold

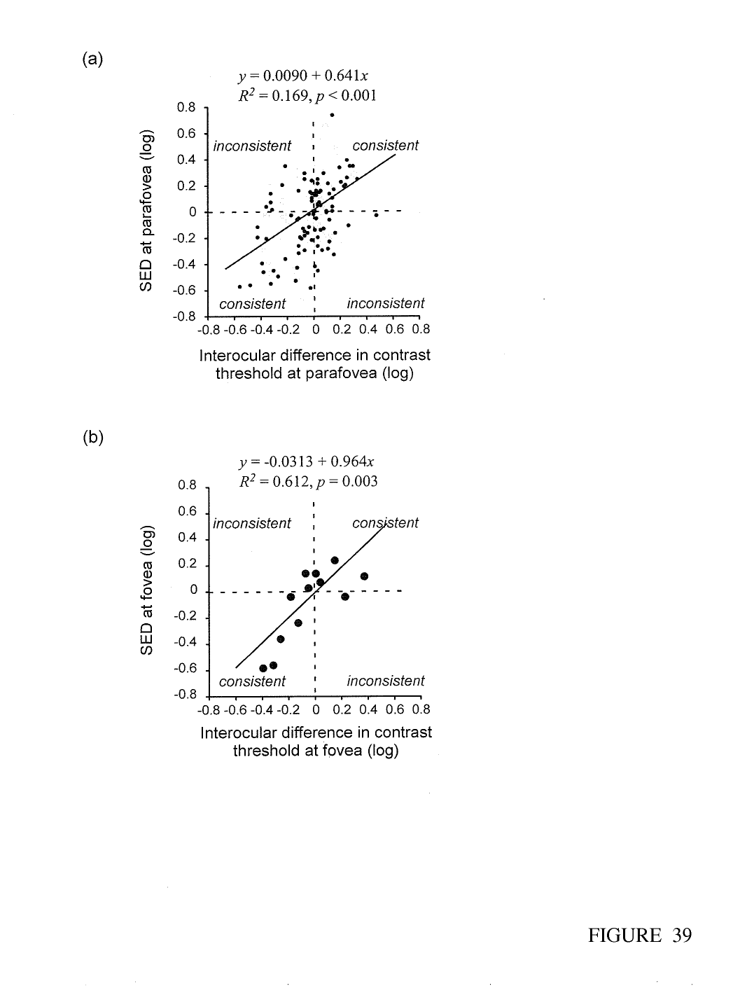

FIG. 39a plots all 12 observers' paired data points between the SED and interocular difference in contrast threshold obtained from the parafoveal region (2°: small black circles; 4°: large gray circles). There are 96 data points (12 observers×8 locations) for each retinal eccentricity. Although there exists a reliable correlation (2°: R²=0.241, p<0.001; 4°: R²=0.1, p=0.002; both 2° and 4°: R²=0.169, p<0.001), it needs to be emphasized that while the majority of the data points fall in the first and third quadrants (where the two measurements consistently reveal the same eye as superior), there is a noticeable number of data points that fall in the second and fourth quadrants (inconsistent quadrants). These latter data points that fall in the inconsistent quadrants indicate that SED cannot be attributed to a difference in interocular contrast threshold. Also correlated were the 12 observers' foveal SED and interocular difference in contrast threshold data. As shown in FIG. 39b, several data points also fall in the second and fourth quadrants. In fact, about 40% of the variability in foveal SED is not accounted for by variations in the difference in interocular contrast threshold (R²=0.612, p<0.003).

To further emphasize the lack of strong correlation, obtained from among the 204 locations tested (12 observers×17 locations), were 110 locations (54%) where the difference in interocular contrast threshold is smaller than 0.1 log unit. It was found that 72 of the 110 locations (67%) have SED larger than 0.1 log unit. Taken together, these data provide further support that SED cannot be fully accounted for by the monocular contrast sensitivity explanation. However, it is important to note that such a conclusion does not exclude the contribution of the interocular difference in contrast threshold to SED. In fact, these findings indicate a partial contribution. Thus, the measured SED may not be entirely caused by an asymmetric gain of mutual inhibition in the interocular inhibitory cortical network.

5. Stereopsis is Affected More by SED than by a Difference in Interocular Contrast Threshold

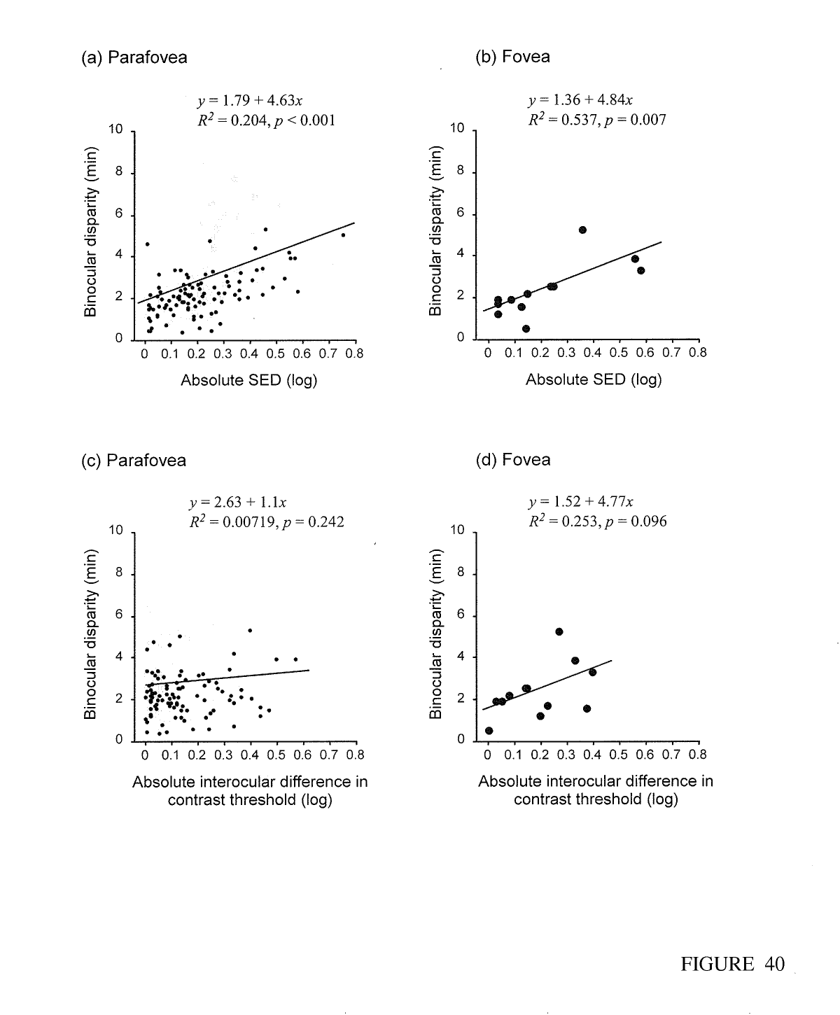

FIGS. 40a and 40b correlate binocular disparity threshold with the absolute value of the SED, respectively, at the parafoveal and foveal locations. Both at the foveal and parafoveal regions, binocular disparity threshold increases with the absolute SED (foveal: R²=0.537, p=0.007; 2°: R²=0.3, p<0.001; 4°: R²=0.222, p<0.001; both 2° and 4°: R²=0.204, p<0.001), suggesting that an imbalance in the interocular inhibitory network can degrade the binocular depth process. The binocular disparity threshold was then correlated with the absolute value of the interocular difference in contrast threshold (FIGS. 40c and 40d). In comparison to SED, there is only a weaker tendency for the binocular disparity threshold to increase with the absolute interocular difference in contrast threshold (foveal: R²=0.254, p=0.096; 2°: R²=0.0203, p=0.166; 4°: R²=0.0337, p=0.073; both 2° and 4°: R²=0.00719, p=0.242). This difference further underscores the important role of the interocular inhibitory mechanism in processing binocular depth information.

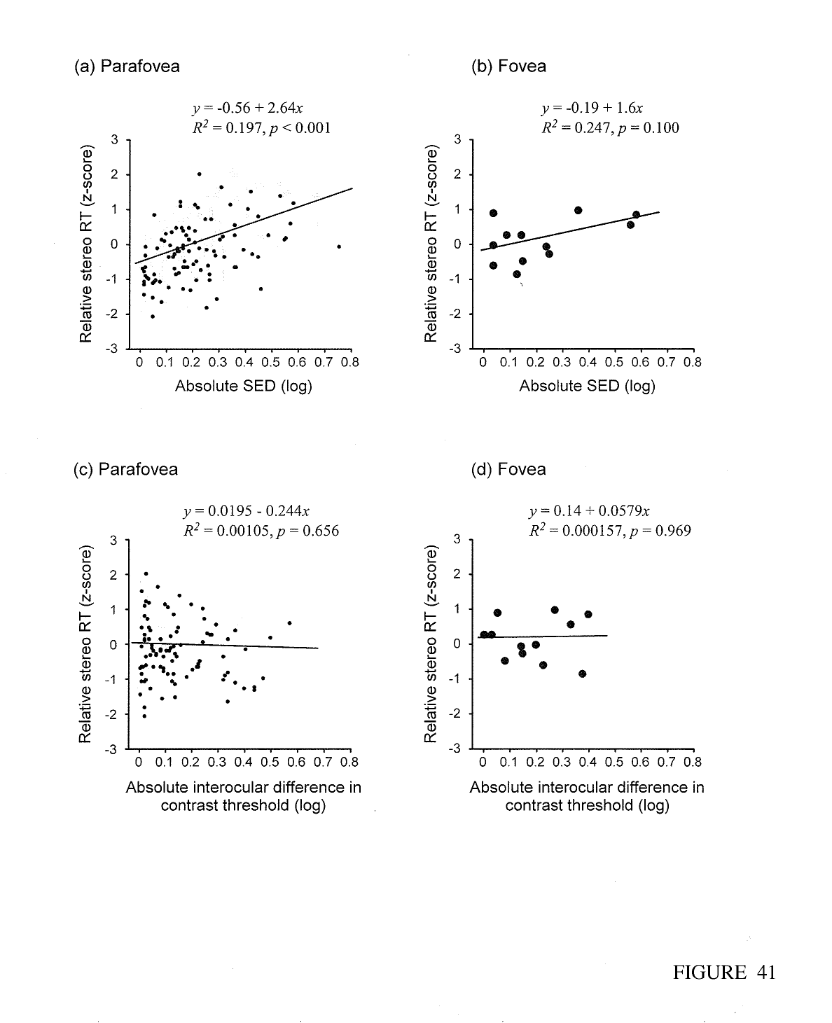

Similar results are found in correlation analyses of the relative reaction time to detect a target in depth (average of crossed and uncrossed disparity trials) with the absolute SED (FIGS. 41a and 41b), and with the absolute interocular difference in contrast threshold (FIGS. 41c and 41d). Relative reaction time to detect a target in depth increases with the absolute SED (foveal: R²=0.247, p=0.100; 2°: R²=0.161, p<0.001; 4°: R²=0.252, p<0.001; both 2° and 4°: R²=0.197, p<0.001), but barely increases with the absolute interocular difference in contrast threshold (foveal: R²=0.000157, p=0.969; 2°: R²=0.011, p=0.310; 4°: R²=0.0153, p=0.230; both 2° and 4°: R²=0.00105, p=0.656).

Also examined were the tested locations in the parafoveal area where the difference in interocular contrast threshold is smaller than 0.1 log unit. It was found that the correlation coefficient between binocular disparity threshold and SED (R²=0.178, p<0.001), and between relative reaction time and SED (R²=0.209, p<0.001), are significant. This further implicates the contribution of the interocular inhibitory mechanism to binocular depth perception.

6. Correlation Between SED and Predominance in Binocular Rivalry with Extended Viewing Duration

The observers' percepts were measured while tracking a 30 sec binocular rivalry stimulus with foveal vision to reveal the dynamics of binocular rivalry (dominance shifts). The data was then correlated with the foveal SED. For each observer, his/her interocular difference in predominance measured in proportion was calculated, which is defined as:

[Predominance(RE,H)−Predominance(LE,H)]+[Predominance(RE,V)−Predominance(LE,V)]

In the above, H and V denote the perceived horizontal and vertical image, respectively.

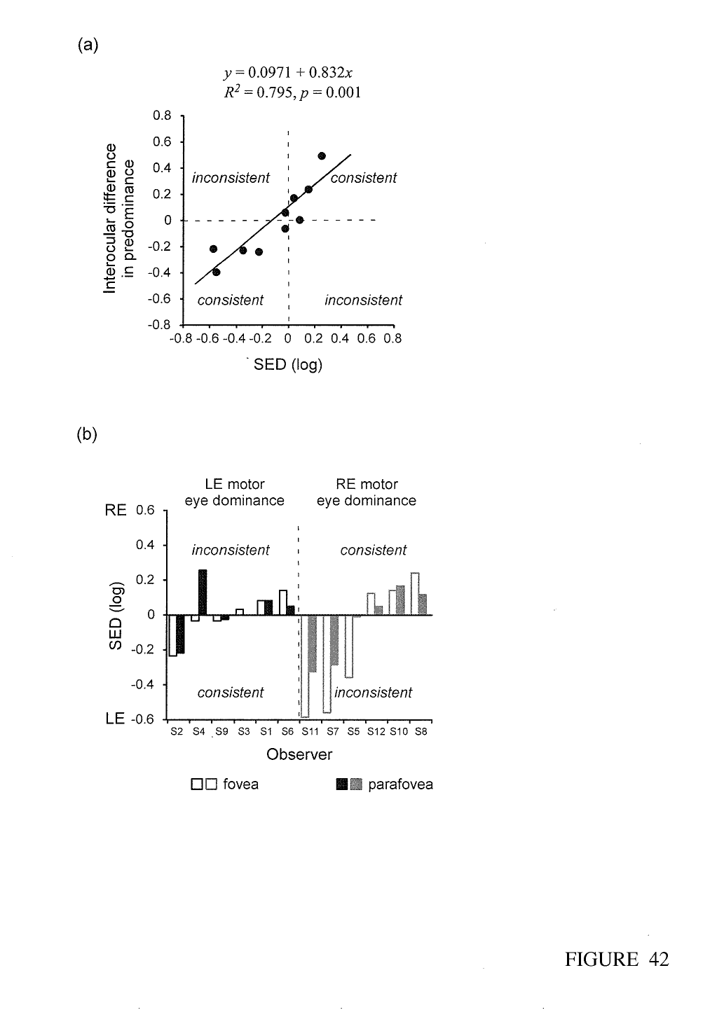

Each observer's interocular difference in predominance was paired with his/her foveal SED and were plotted in FIG. 42a. The graph reveals a strong correlation (R²=0.795, p=0.001). Data falling in the first and third quadrants indicate the superior eye in SED also enjoys a higher predominance when tracking binocular rivalry. Nevertheless, one of the ten observers tested had her data point falling in the second quadrant; however, it is likely that this data point is affected by the fluctuation of the visual system's intrinsic noise level since both measurements are rather small in magnitudes. Whether the foveal SED has a similar relationship with the dominance duration and dominance frequency in the binocular rivalry tracking task was also examined. It was found that reliable correlation between SED and dominance duration (R²=0.6403, p=0.005) exists, but not between SED and alternation frequency (R²=0.0023, p=0.894). Overall, it was concluded that there is a general agreement between eye dominance based on the SED task and that based on the binocular rivalry tracking task.

7. A Non-Significant Correlation Between Sensory and Motor Eye Dominance

This study supports that the underlying mechanisms of SED and motor eye dominance (MED) are different. The observers' foveal and parafoveal SED were compared with MED. The parafoveal SED result for each observer was obtained by averaging his/her SED data from the 2° and 4° eccentricities. FIG. 42b depicts the 12 observers' SED and MED data. Clearly, there are at least four observers whose data (both foveal and parafoveal) fall in the inconsistent regions. This indicates that eye dominance is independent for the motor and sensory modalities.

### Example 6

### Push-Pull Protocol as a Treatment for Amblyopia

A. Experimental Procedures

A Macintosh computer running Matlab and Psychophysics Toolbox generated the stimuli on a flat 21″ CRT monitor (2048×1536 pixels at 75 Hz). The observers viewed the monitor through a mirror haploscope attached to a head-and-chin-rest from a distance of 100 cm. The experiments performed conformed with the regulatory standards of the Institutional Review Board of Salus University.

Subjects:

Three adults (25-38 years old) with amblyopia participated in the study (visual acuity: S1: RE=20/20, LE=20/50⁻²; S2: RE=20/25⁻¹, LE=20/16⁻²; S3: RE=20/16⁻², LE=20/63⁻²). All had previously been diagnosed and treated by their Optometrists but were no longer undergoing treatment at the time of the study. The origins of their amblyopia (based on self-reported history) were strabismus for S2 and anisometropia and strabismus for S1 and S3. All observers were able to achieve binocular eye alignment at the time of testing.

Procedures:

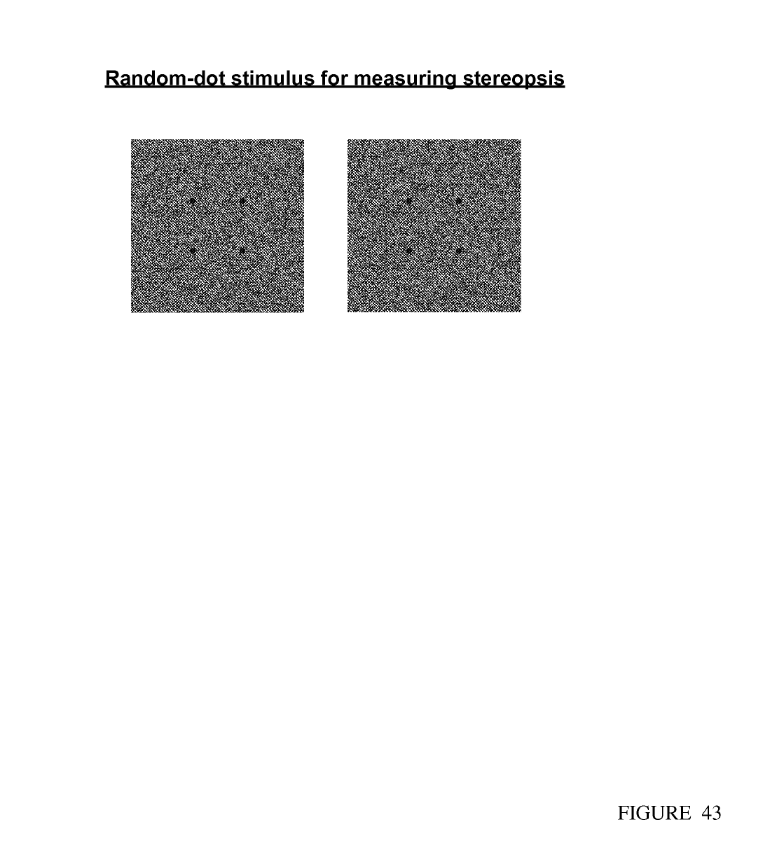

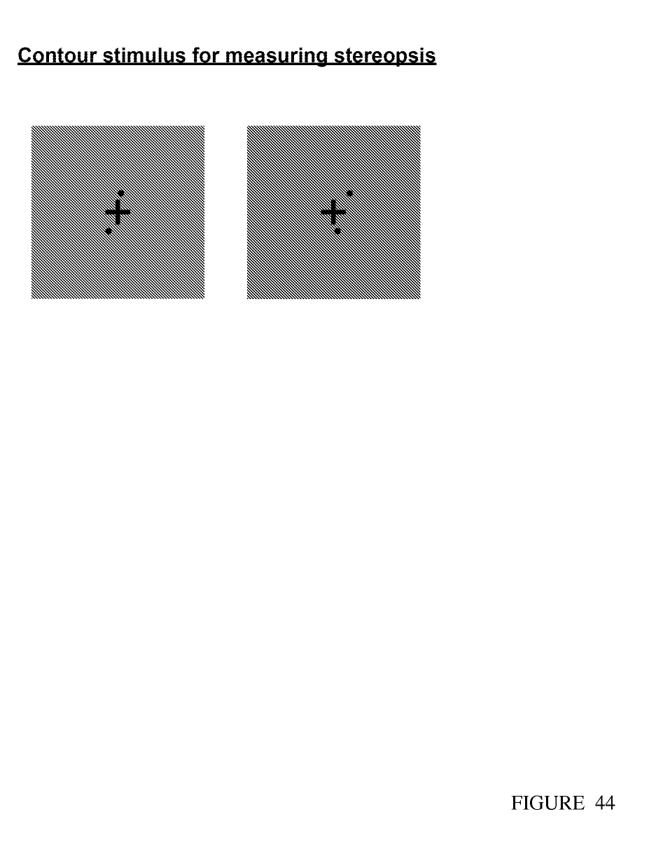

A series of pre- and post-training tests were conducted, including SED, monocular contrast threshold and stereopsis (threshold and reaction time) tests. While the same tests were used, the specific test variables were customized for each observer. This is because amblyopia affects each individual differently resulting in different degrees of amblyopia, particularly on SED and stereo ability. For example, S2 was tested with random-dot stereogram (FIG. 43) whereas S1 and S3 were tested with contour stereogram (FIG. 44). (It has been found clinically that it is more difficult for amblyopes to perceive depth with random-dot stereogram.) Observers S1 and S2 underwent 15 training sessions while S3 underwent 7 training sessions. Two psychophysical tasks, orientation discrimination and contrast discrimination, were used to implement the push-pull protocol during the training. The specific designs of the push-pull protocol were also customized for individual amblyope. To monitor the progress of learning, SED was measured before and after each training session. The detailed stimulus designs of the various tests are described below.

1. SED Test

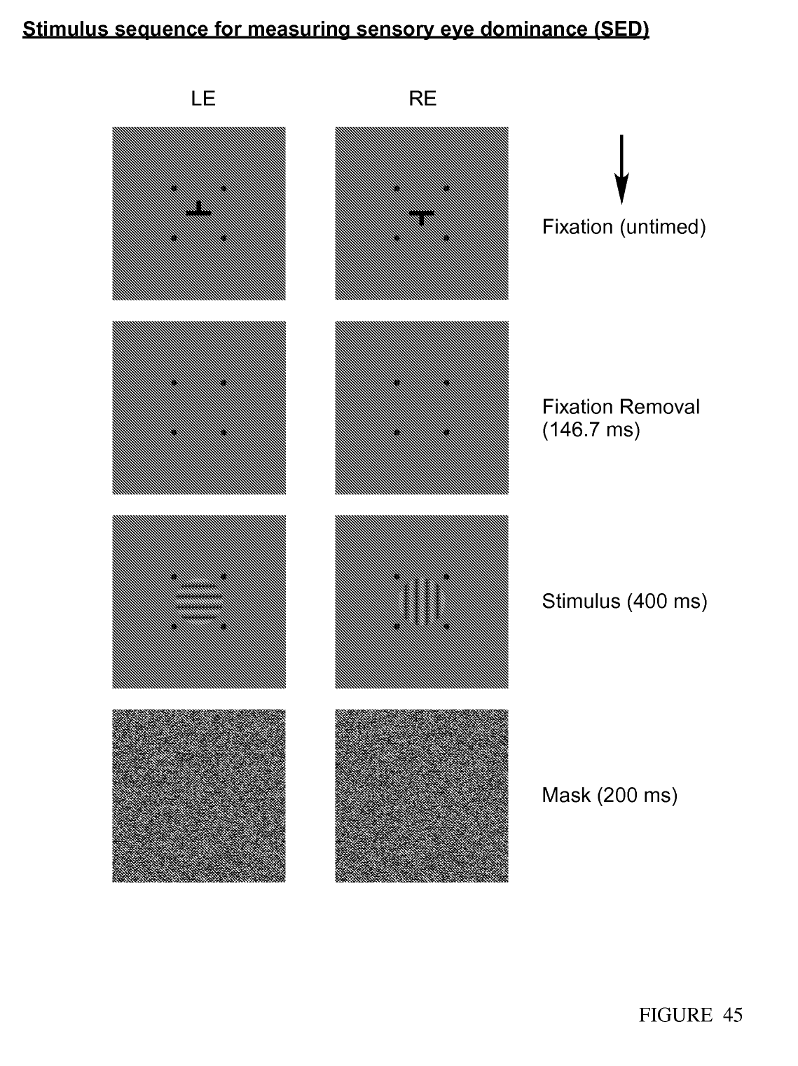

Foveal SED was measured by varying the vertical grating contrast while fixing the horizontal grating contrast constant. The gratings were 1.5° (angular diameter) discs presented to the fovea. During a “generic” SED test trial, the dichoptic orthogonal gratings (vertical vs. horizontal) were presented for 400 msec, followed by a 200 msec mask (black and white random noise pattern made of 0.083° squares, 35 cd/m², 1.7 log % contrast) that terminated the trial (FIG. 45). The observer responded to his/her percept, horizontal or vertical, by pressing the appropriate key. If he/she saw a mixture of the two gratings, he/she would respond to the predominant orientation perceived. A QUEST procedure was used to adjust the vertical grating contrast according to the observer's response, while the horizontal grating contrast remained the same (reference contrast). By appropriately adjusting the grating contrast after each trial, the point of equality, where the observer obtained an equal chance of seeing the two gratings (equal predominance) was reached. The contrast obtained defines the balance contrast for the eye that viewed the variable contrast vertical grating. The gratings were then switched between the two eyes, to obtain the balance contrast for the fellow eye. Five such blocks of trials were run to obtain the mean balance contrast for each eye in the pre- and post-training test phases. The difference between the LE and RE mean balance contrast values is defined as the SED. SED was also measured during the training phase, twice before and twice after each training session. This generic SED protocol, which was also used to measure SED in the non-amblyopic observers, was augmented for testing the amblyopic observers. This is due to the excessive SED in amblyopia, which requires the SED protocol to be customized for individual observer. Below, the specific customization implemented for each amblyopic observer is described, pertaining to the contrast and luminance of the stimuli presented to the two eyes.

First, the fixed contrast of the horizontal grating was held at different levels when presented either to the RE or LE. For observer S3, the contrast of the horizontal grating to the amblyopic LE was fixed at 1.95 log unit when the RE was tested with the variable vertical grating. Then when the LE was tested with the variable vertical grating, the horizontal grating contrast in the RE was fixed at 0.4 log unit. Second, observer S3's strong eye viewed its half-images with reduced luminance that was equivalent to viewing through a neutral density filter with 20% transmission. This is to further reduce the strength of the sensory dominant eye.

In addition to having different reference contrast for each eye, often, the balance contrast of the weak eye might be transiently elevated during a training session. This occurred for observers S1 and S2. For S1, the contrast of the horizontal grating to the amblyopic LE was fixed at 1.8 log unit when the RE was tested with the variable vertical grating. Then when the LE was tested with the variable vertical grating (before each training session), the horizontal grating contrast in the RE was fixed at 0.6 log unit for training sessions 6-9 and 0.8 log unit for the remaining sessions. The horizontal grating contrast in the RE was also fixed at 0.6 log unit when the SED was tested after each training session. For S2, the horizontal grating contrast was 1.3 log unit for both eyes before each training session, but was changed to 1.1 log unit in the LE when measured after each training session.

2. Monocular Contrast Threshold Test

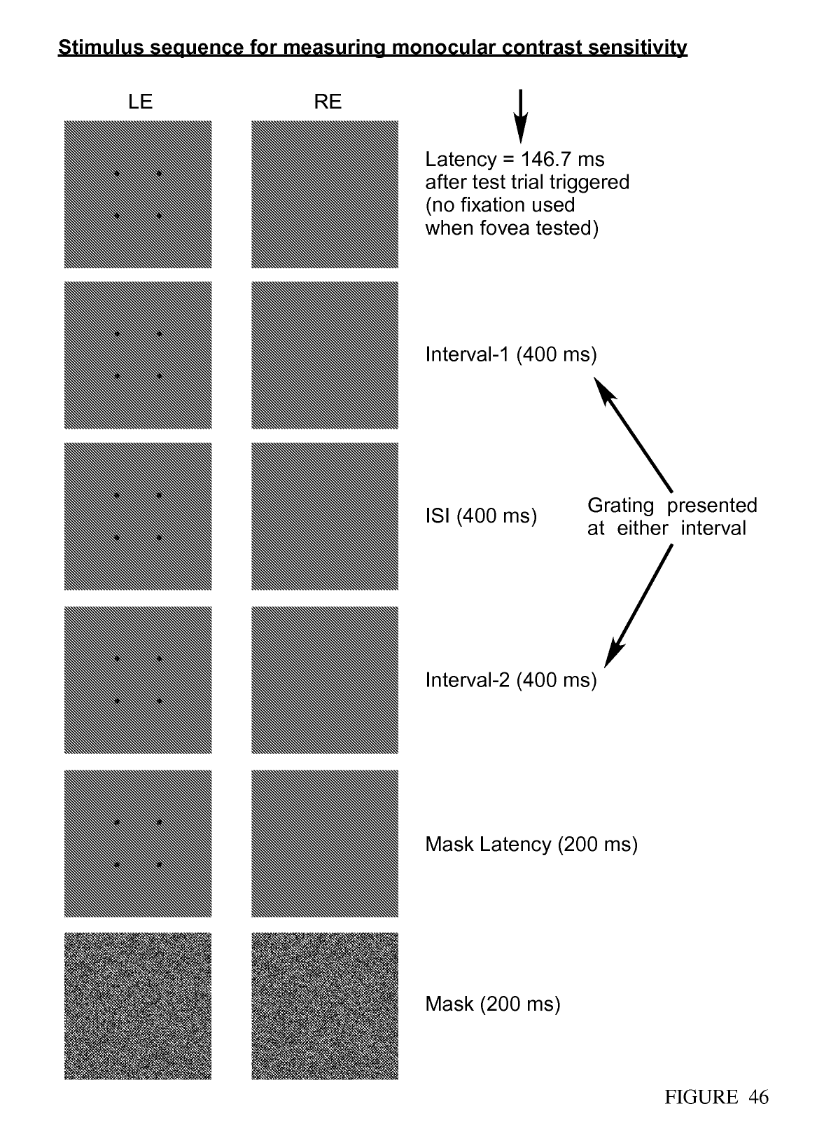

The monocular vertical sinusoidal grating (35 cd/m², 3 cpd, 1.5°) was presented to the test eye, while a homogeneous gray (blank) field with the same mean luminance level was presented to the fellow eye. Each test was conducted using the 2AFC method, whose temporal sequence was: fixation, interval-1 (400 msec), blank (400 msec), interval-2 (400 msec), blank (200 msec), and mask (8°×8° black and white random noise pattern made of 0.083° squares, 35 cd/m², 1.7 log unit, 200 msec) (FIG. 46). A grating disc was presented at only one interval while the other interval had a blank field. The observer responded whether he/she saw the grating in either interval-1 or -2 by a key press. Grating contrast was adjusted after each trial (by QUEST) to obtain threshold. This contrast threshold test was repeated four times in each eye, both in the pre-training and post-training phases.

3. Stereo Threshold Test

Owing to differences in the amblyopic observers' stereo ability, S2 was tested with random-dot stereogram while S1 and S3 with contour stereogram. The random-dot stereogram (8°×8°, dot size=0.025°, 35 cd/m², contrast=1.0 log %) had a variable crossed-disparity disc target (1.5°) (FIG. 43). The contour stereogram comprised of two dots (0.2°, separated by 1.2° vertically, 2.3 cd/m²) with variable crossed-disparity against a 35 cd/m² background, one above and the other below the fixation (0.8°×0.8°) (FIG. 44).

For both types of stereograms, the standard 2AFC method in combination with the staircase procedure was used to measure the stereo disparity threshold. The temporal sequence of the stimulus presentation was interval-1, blank (400 msec), interval-2, blank (200 msec), and random-dot mask (200 msec, 8°×8°, 35 cd/m², 1.7 log %, 0.083° squares). The durations of interval-1 and interval-2 were individually specified (1.6 sec for S1, 200 msec for S2 and S3). After each trial, the observer indicated whether the crossed-disparity disc (front) was perceived at interval-1 or -2. Each test block comprised 10 reversals (step size=0.67 arc min after two initial trials, total ˜40-80 trials), and the last 6 reversals were taken as the average threshold. Each block was repeated five times.

4. Stereo Response Time Test

Stereo reaction time was measured for observers S2 (with random-dot stereogram, FIG. 43) and S3 (with contour stereogram, FIG. 44). The stereo target (disc for random-dot stereogram or two dots for contour stereogram) was above the observers' stereo threshold and rendered with either crossed or uncrossed binocular disparity (S2: ±9.62 arc min; S3: ±16.67 arc min). Catch trials with zero binocular disparity were randomly inserted during the testing (each block of trials had 40 test trials and 10 catch trials). During a trial, the observer pressed a key immediately upon detecting the stereo target (1=front, 2=back). A 200 msec mask followed to terminate the trial (8°×8°, 35 cd/m², 1.7 log %, 0.083° squares). If no target was seen, he/she did not need to press any key. To ensure performance accuracy, the computer was set to terminate a block of trials if the observer exceeded 20% false alarm rate. Responses of trials shorter than 100 msec were rejected as anticipatory responses, and the test program automatically repeated these trials. When calculating the mean RT, responses violating the 3-SD rule were omitted. This did not occur often (<1% occurrence).

5. Push-Pull Training Protocol

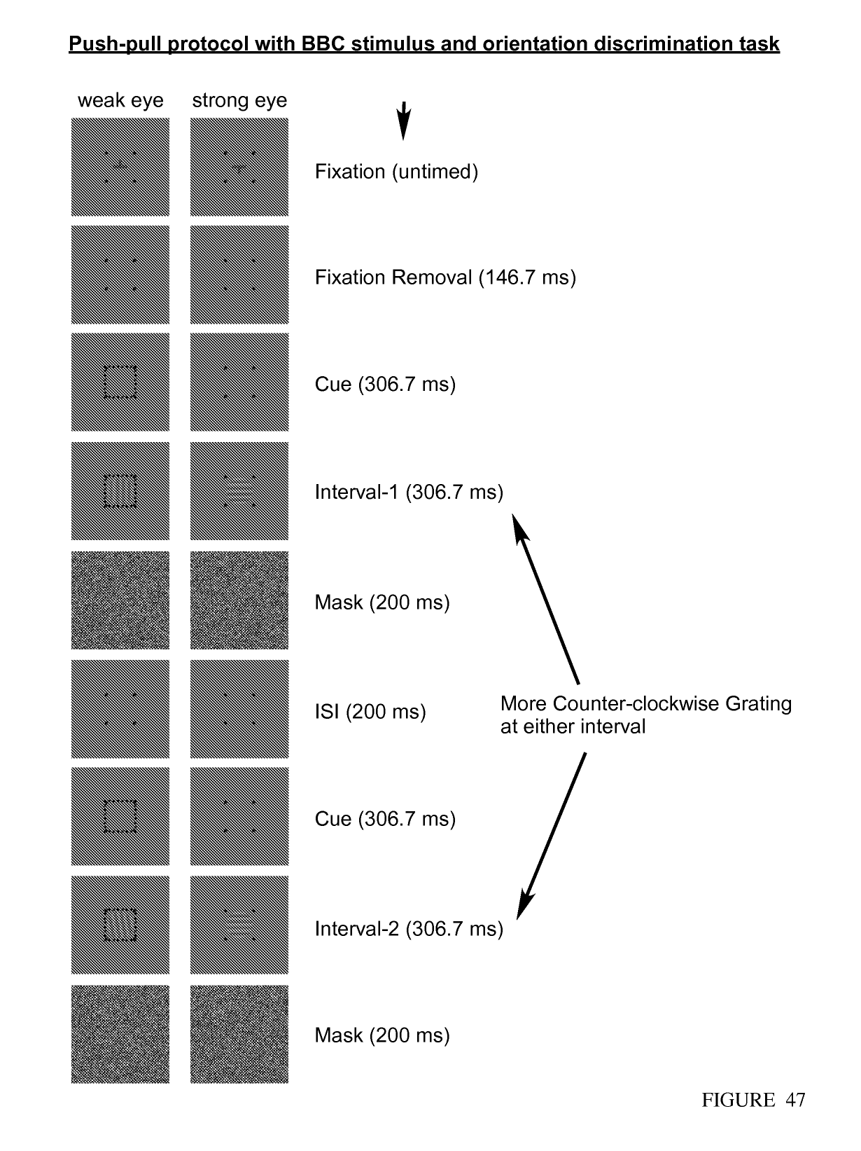

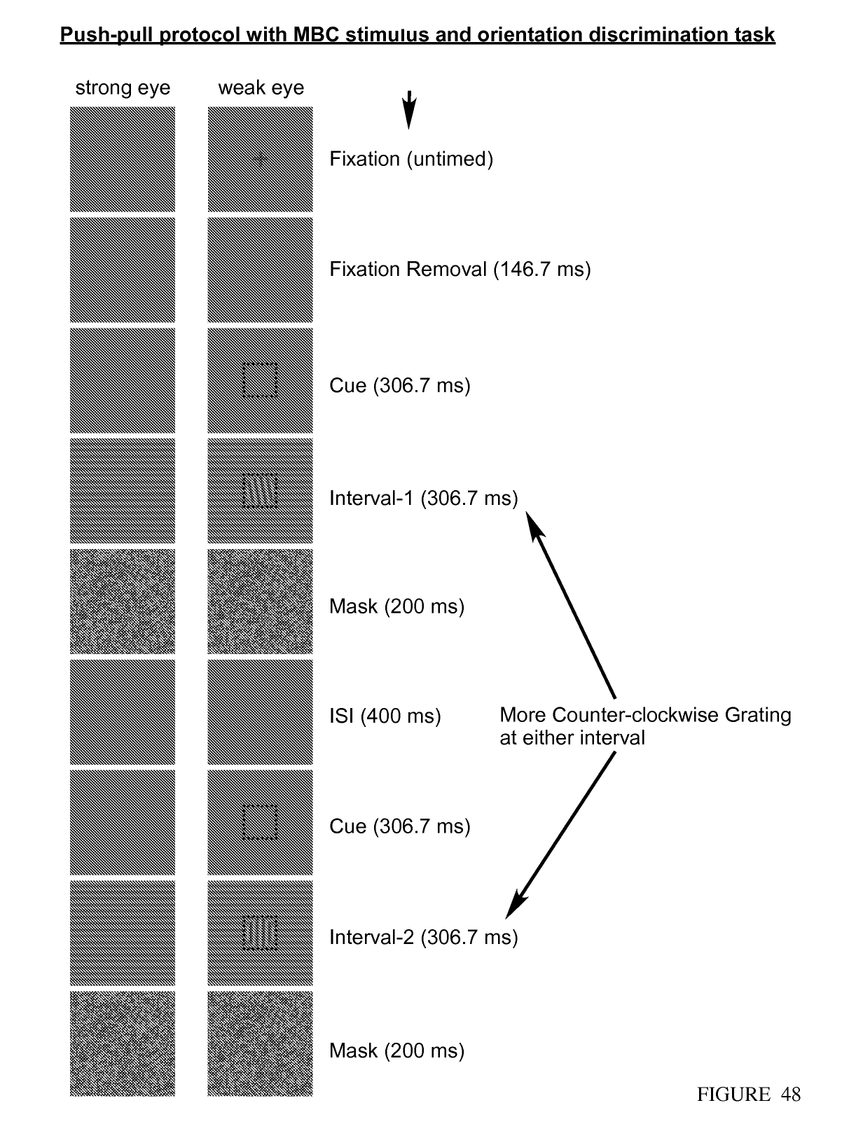

Observers S1 and S2 were trained on a push-pull protocol with a binocular boundary contour (BBC) target comprising of a pair of dichoptic orthogonal gratings (306.7 msec, 1.5°, 3 cpd, 35 cd/m²) (FIG. 47). Meanwhile S3 was trained with a monocular boundary contour (MBC) target comprising of a MBC disc (306.7 msec, 1.5°, 3 cpd, 35 cd/m²) viewed by the weak amblyopic eye and a homogeneous grating (17.2°×11.5° in each half-image, 3 cpd, 35 cd/m²) viewed by both eyes (FIG. 48).

To begin a 2AFC training trial for either the stimulation in FIG. 47 or 48, the observer aligned his/her eyes at the fixation target (0.8°×0.8°, line width=0.15°, 15 cd/m²), and then pressed the spacebar on the computer keyboard. This led to the removal of the fixation target, which was replaced by the presentation of a monocular square frame (2.3 cd/m², 1.5°×1.5° dash outline with frame width of 0.15° for S1, 1.85°×1.85° dash outline with frame width of 0.12° for other observers) in the weak amblyopic eye. The interval between the removal of the fixation and the onset of the square frame was 146.7 msec. The square frame acted as a transient attention cue to attract attention to the vicinity of the cue in the weak amblyopic eye (Ooi & He, 1999). After a 306.7 msec presentation of the cue (cue-lead-time), the BBC target (for S1 and S2, FIG. 47) or MBC target (for S3, FIG. 48) was presented for 306.7 msec, followed by a random-dot noise mask (200 msec; 35 cd/m²; 1.7 log %; 17.2°×11.5° for S3, 8°×8° for other subjects) (end of interval-1 of the 2AFC trial). The same 306.7 msec cue was presented again 200-400 msec later, followed by the presentation of a second pair of BBC or MBC target (306.7 msec), and a 200 msec mask presentation to terminate the trial. The grating shown to the weak amblyopic eye in this second presentation had a slightly different orientation from the grating shown in the first presentation. The observer's (orientation discrimination) task was to report by a key press whether the first or second grating had a slight counterclockwise orientation. This ended a training trial. Orientation discrimination threshold was obtained by appropriately adjusting the orientation of the subsequent trials using either the QUEST or staircase method.

Notably, the attention cueing technique was slightly different from the one used with the non-amblyopic observers in that the cue in the present technique remained with the stimulus. For example, compare FIG. 47 with FIG. 2. Two other aspects of the stimulation were also different from those used with the non-amblyopic observers. These pertain to the grating contrast and augmentation of the stimulus to the weak (amblyopic) eye, as elaborated below.

Grating Contrast:

Initially, the contrast values of the dichoptic gratings used in the training were those that led to the points of equality in the RE and LE with the SED test obtained before the training phase. However, unlike the push-pull training previously implemented on non-amblyopic observers, the contrast values of the dichoptic gratings for the amblyopes were modified as training progressed such that the weak eye received lower contrast and strong eye higher contrast, i.e., making it more difficult for the weak eye to maintain its dominance (but still succeed). In other words, “pull harder” on the strong eye.

Enhancing Signals in the Weak Eye (“Push Harder”) to Ensure its Dominance During the Training:

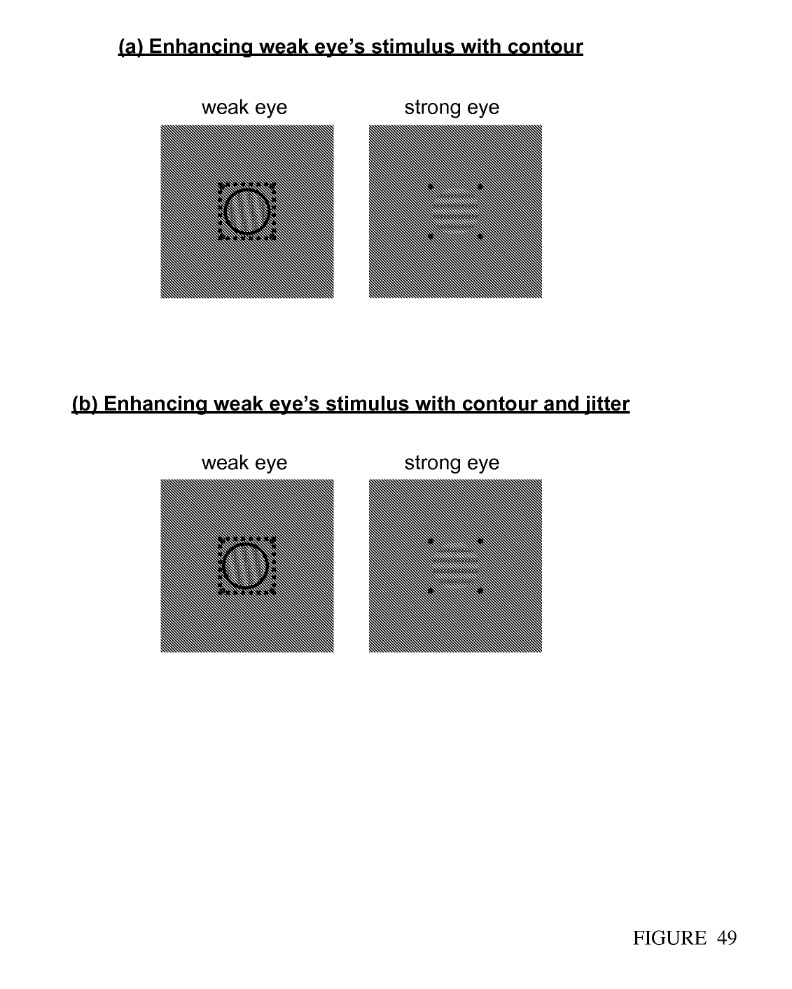

Furthermore, to promote dominance of the weak eye, the half-image viewed by the weak eye were sometimes augmented with contour ring (0.1°), jitter (range: ±0.1°, speed: 4° per sec, temporal frequency: 5 Hz) and counterphase motion (speed: 4° per sec, temporal frequency: 5 Hz). Refer to the examples in FIGS. 49a and 49b. Finally, for the training with the MBC stimulus (for observer S3), attention cueing was also added to ensure that the weak eye became dominant. This is different from the procedure used on non-amblyopic observers, where attention cueing was not added to the protocol. Compare FIG. 48 with FIG. 28b.

Each block of trials to obtain threshold comprised about 50 trials. Multiple blocks lasting about 1.5 hours were run during a training session (S1: 18 blocks; S2: 15 blocks; S3: 9-15 blocks). Observers S1 and S2 underwent 15 training sessions while observer S3 underwent 7 training sessions.

A Variant of the Task Used in the Push-Pull Protocol:

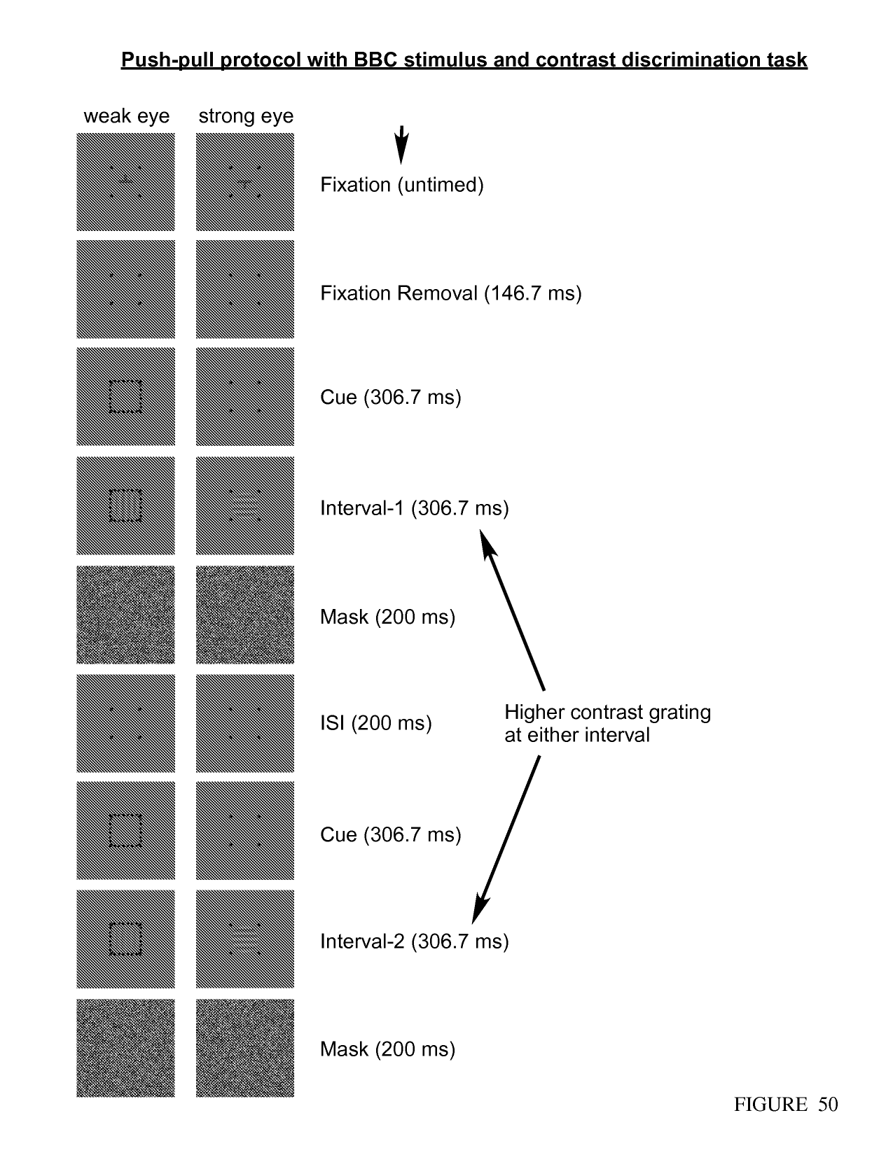

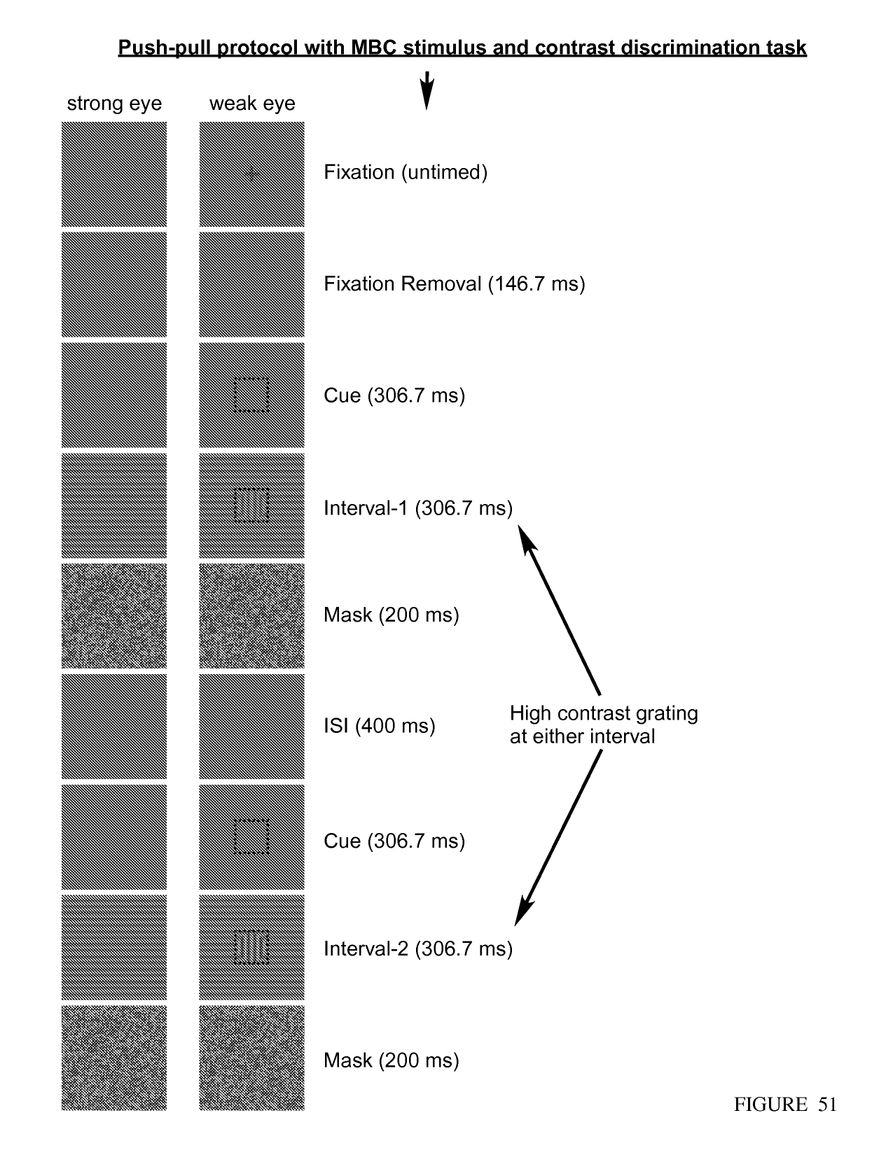

To offer variety, separate push-pull protocols were also designed that required observers to perform a contrast discrimination task, instead of the orientation discrimination task. The stimulus variables were the same as those above, except that the dominant gratings in the weak eye varied in contrast rather than orientation (FIG. 50 for S1 and S2 and FIG. 51 for S3). The observers selected the 2AFC interval with the higher grating contrast. All other aspects of the training, including grating contrast manipulation and stimulus enhancement factors were the same as in the push-pull with orientation discrimination task.

During each training session, 3 blocks of orientation discrimination task were interleaved (alternated) with 3 blocks of contrast discrimination task.

B. Results

1. Primary Findings (Effects of Training on Binocular Functions)

a. Effect of Training on SED

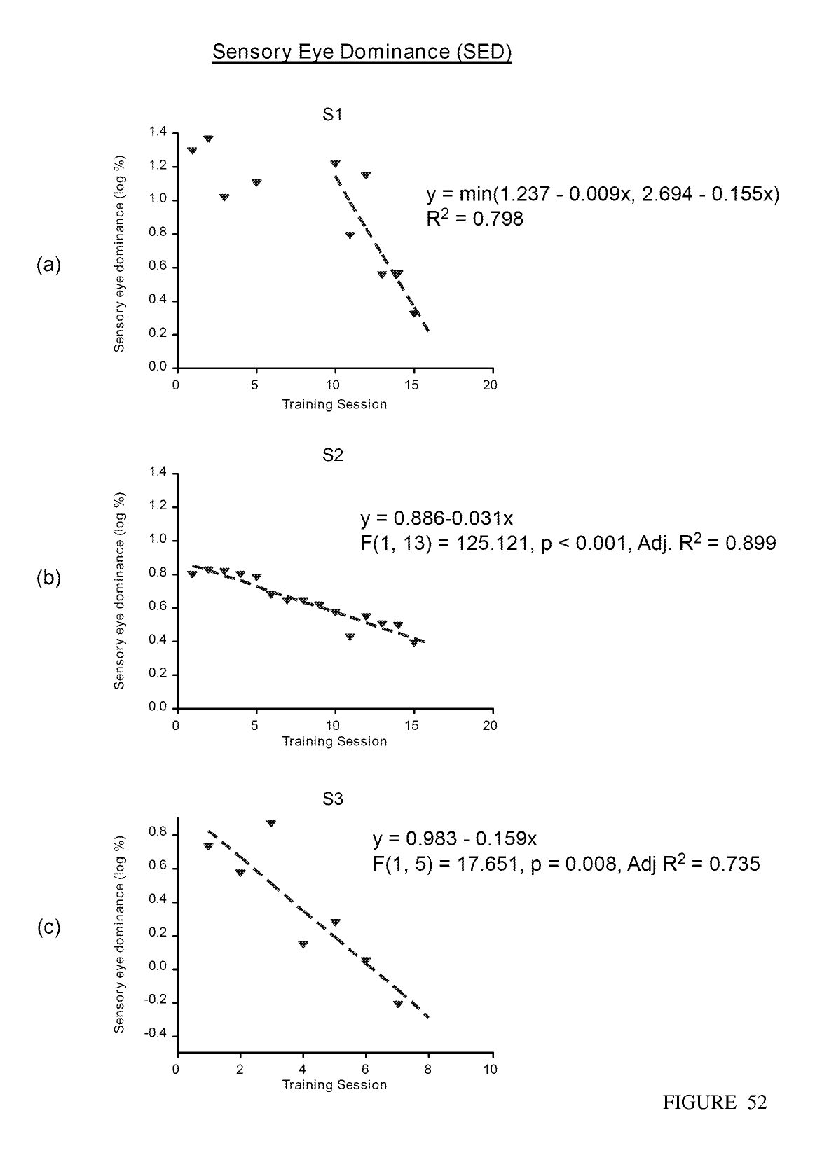

To monitor the changes in SED over the multiple training sessions, the observers' SED was measured before and after each training session. FIGS. 52a, 52b and 52c show the changes in SED after each training session, respectively, for observers S1, S2 and S3. Clearly, the magnitudes of SED became smaller as the push-pull training progressed. (The relevant statistical analyses for this and the other results below are presented with the graphs in the figures.)

It is worth noting that while the reduction in SED for observer S3 appears to cause the non-amblyopic eye to be excessively weakened (negative SED), it should not be interpreted as such. Because of this amblyope's large magnitude of SED, observer S3's non-amblyopic eye was disadvantaged in two respects in order to obtain SED. This was done by reducing the overall luminance of the stimulus and lowering the reference contrast of the horizontal grating in the non-amblyopic eye. Therefore, while the data show a significant improvement in SED reduction after the training, the amblyopic eye was nevertheless still weaker than the non-amblyopic eye and will likely benefit from extended push-pull training.

Overall, the findings from all three observers indicate that the push-pull protocol is effective in reducing SED in the amblyopic population.

b. Effect of Training on Stereo Threshold

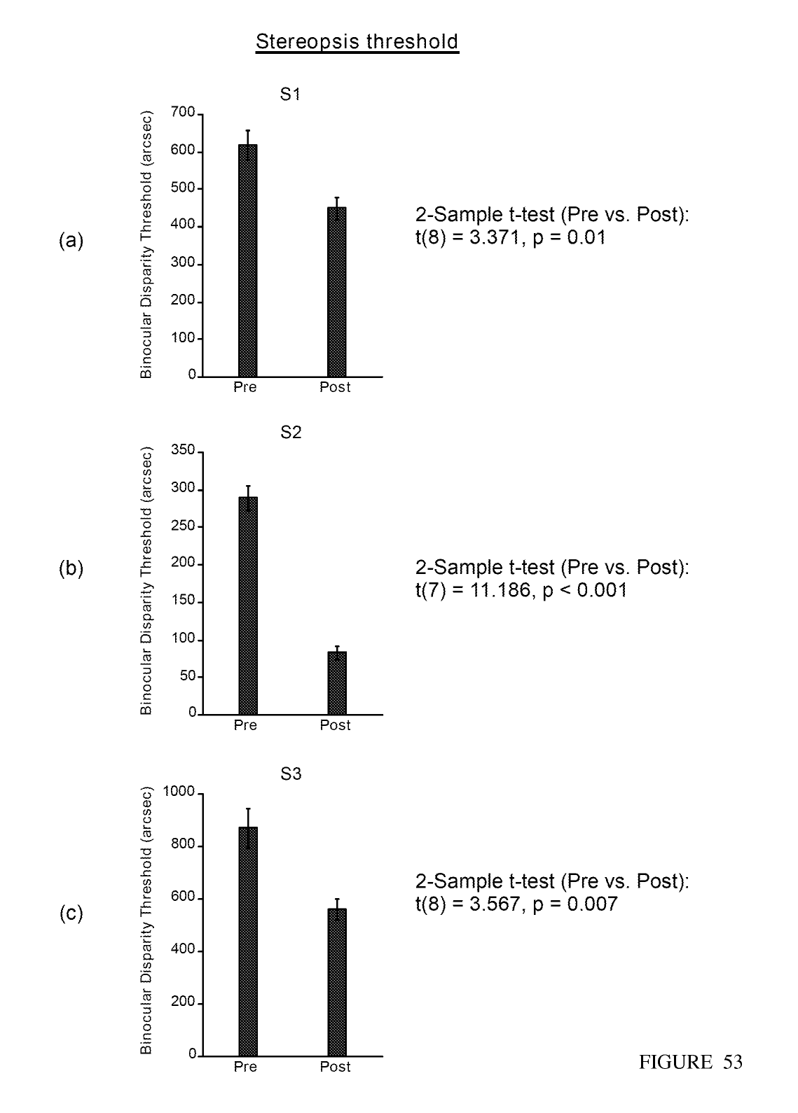

S1 and S3 were tested with contour stereogram because they were incapable of experiencing stereopsis with random-dot stereogram. S2 was tested with random-dot stereogram. FIGS. 53a, 53b and 53c plot the stereo thresholds of observers S1, S2 and S3, respectively, before and after the push-pull training. Thresholds were decreased post-training by 160-300 arc sec, indicating improved stereopsis after the training. The improved stereopsis is remarkable because the observers were not trained on the stereo stimuli during the training phase.

c. Effect of Training on Stereo Response Time

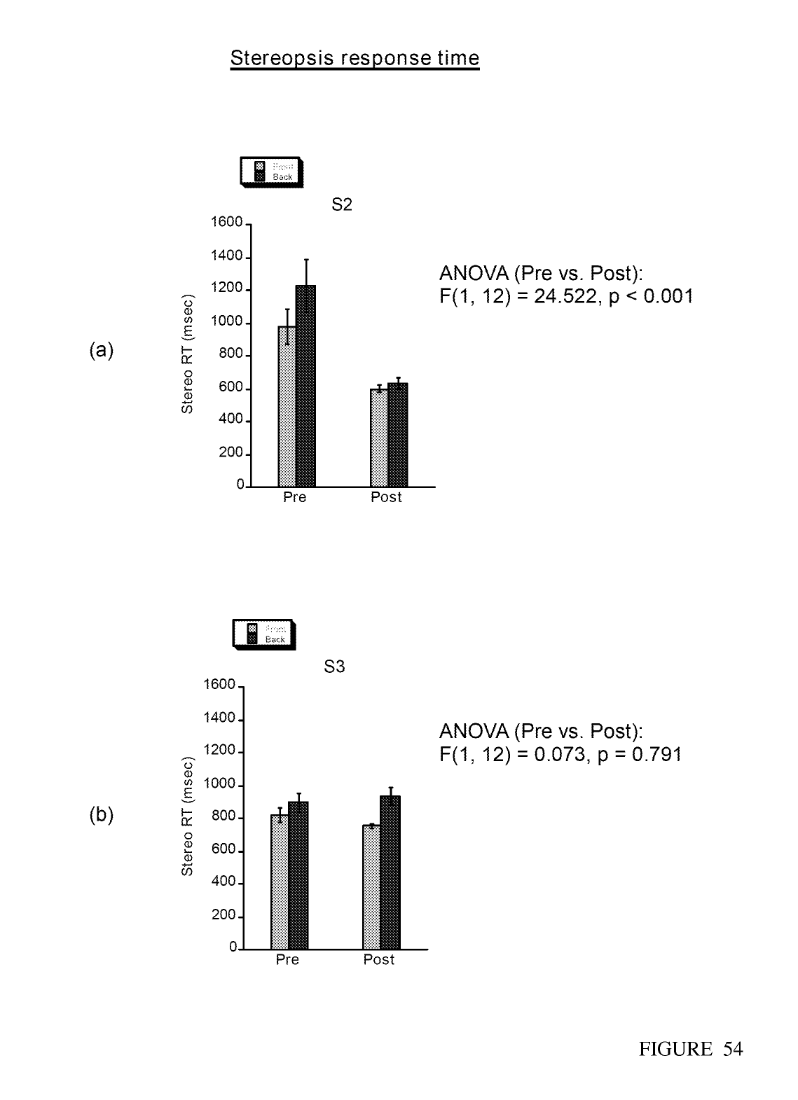

S2 and S3 were also tested for the speed of perceiving a target in depth either in the back or in front. S2's response times (RT) to seeing depth reduced significantly after the training (FIG. 54a). However, this was not the case for S3 even though her depth detection threshold improved (FIG. 54b). This could be because she underwent a shorter training period (7 sessions) and had deeper amblyopia. Possibly, a similar improvement would be observed in S3 had the training period been extended.

2. Secondary Findings (Effects of Training on Monocular Functions)

Because the push-pull protocol recalibrates the balance of excitatory and inhibitory interactions, some improvements were observed in monocular functions of the amblyopic eye. Recall that monocular functions showed less remarkable changes than SED changes after training in the non-amblyopic observers.

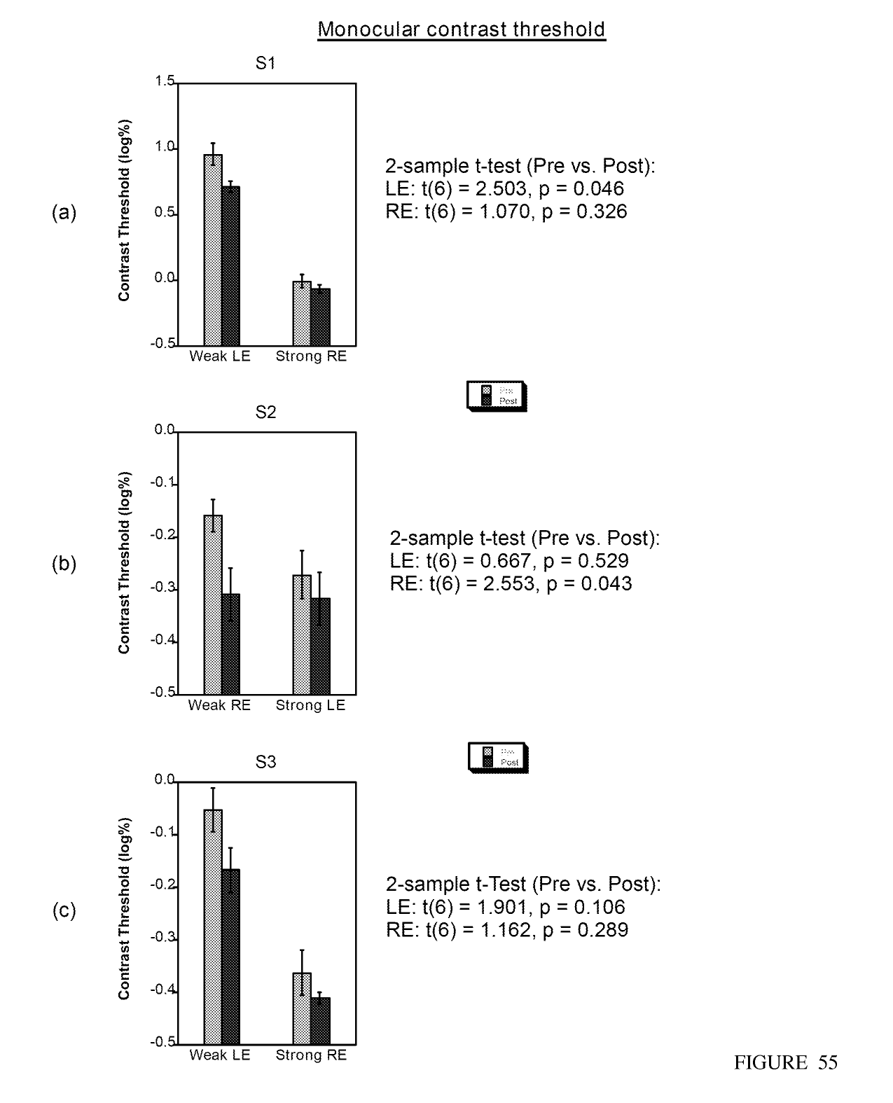

a. Effect of Training on Monocular Contrast Sensitivity

Whereas the monocular contrast sensitivity in the two eyes of non-amblyopic observers are quite equal, this is not so in amblyopia. The amblyopic eye usually exhibits significant deficit in contrast sensitivity. FIG. 55 plots the contrast threshold data of the three subjects. Monocular contrast thresholds are significantly reduced in the weak amblyopic eyes of observers S1 (FIG. 55a) and S2 (FIG. 55b) after the push-pull training, but not significantly reduced for observer S3 (FIG. 55c).

The significant improvement in contrast sensitivity of S1 and S2's amblyopic eyes probably reflects a secondary learning effect of the push-pull protocol. But it cannot entirely account for the reduction in SED.

b. Effect of Training on Amblyopic Eve's Monocular Contrast and Orientation Discrimination Thresholds

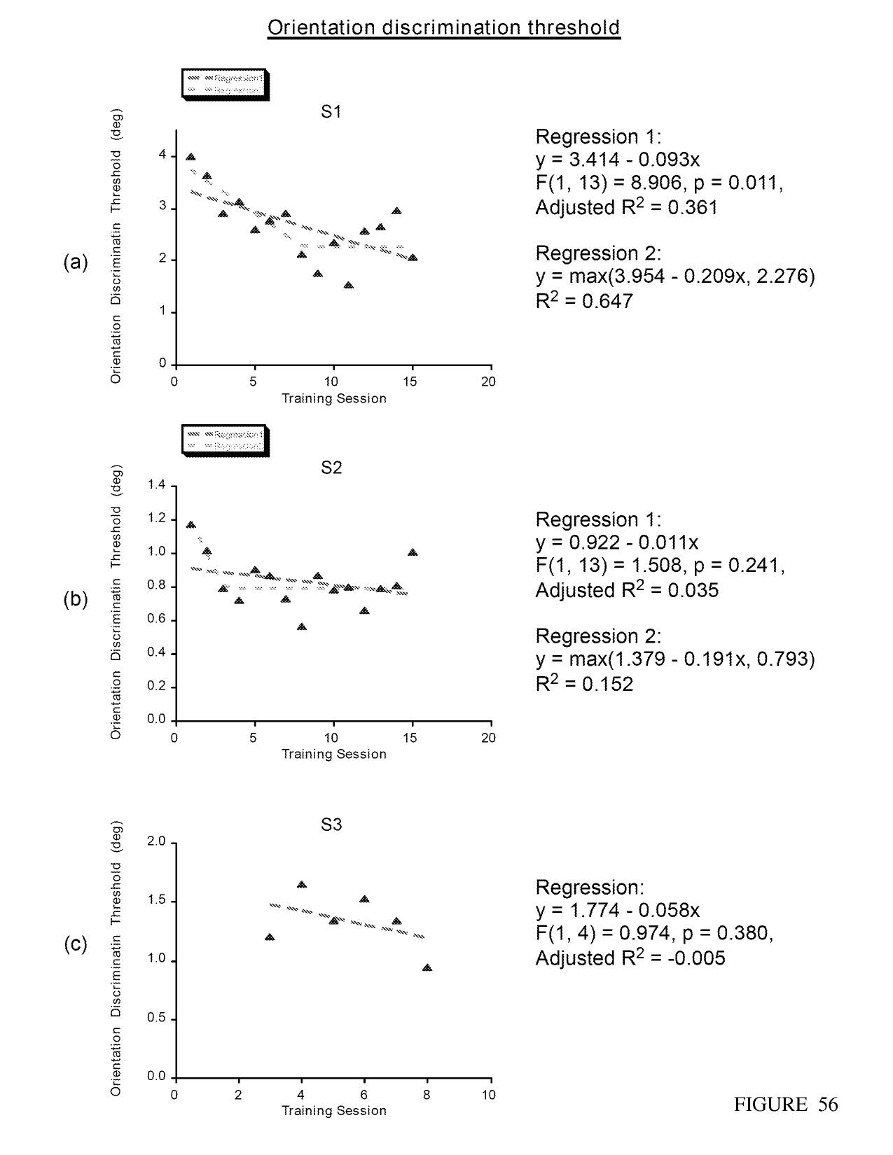

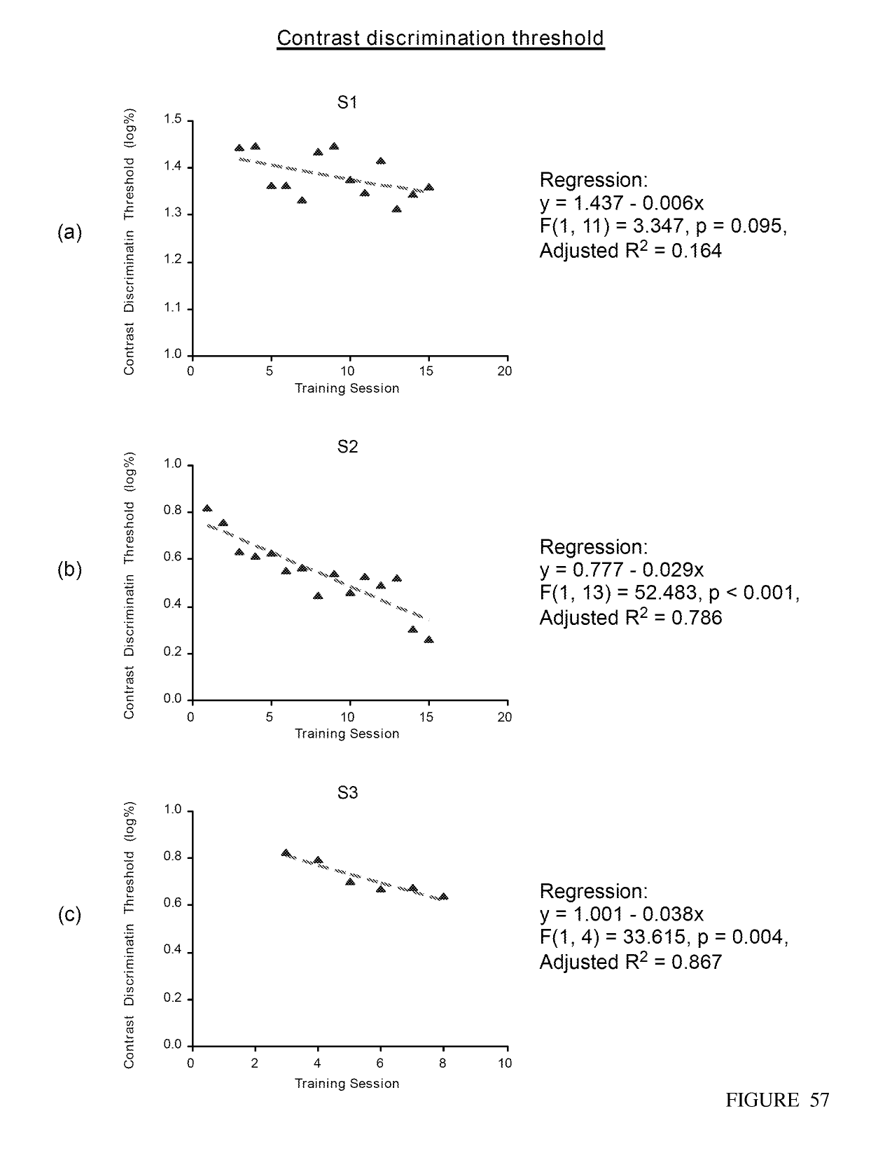

While the primary focus of the push-pull protocol is to balance the mutual inhibition between the two eyes' channels to reduce SED, the design of the push-pull protocol requires obtaining either the discrimination threshold of orientation or contrast during the training phase. In other words, the observers were also exposed to training of orientation and contrast discrimination tasks. FIGS. 56 and 57 show, respectively, the changes in orientation and contrast discrimination thresholds of the weak eye over the multiple training sessions. The data of observers S1, S2 and S3, are presented respectively, in graphs (a), (b), and (c).

As mentioned in the Experimental Protocol section, the contrast of the gratings used in the push-pull method were continually monitored and changed during the training phase to challenge the interaction between the two eyes (“pull harder” on the strong eye). It was reasoned that it is harder to suppress a stimulus with a higher contrast. Therefore, at the start of the training, the grating contrast in the weak eye was higher while the grating contrast in the strong eye was lower. But as training progressed, the tendency was to raise the contrast of the grating in the strong eye and lower the contrast of the grating in the weak eye, while ensuring that the weak eye remained dominant. Because of this modification in our push-pull protocol, the data points used for plotting the orientation and contrast discrimination thresholds as a function of training session (FIGS. 56 and 57) reflect the average thresholds from multiple grating contrast levels that were exposed to the weak eye during a given training session. A regression analyses was performed on the data and a general trend was found in which the discrimination thresholds reduced as the training progressed. The extents of improvements vary among subjects, as would be expected from the fact that amblyopia affects each individual differently. This is evident, for example, when the contrast discrimination thresholds was normalizedrelative to the pedestal contrast (Weber's fraction).

3. Retention of Learning

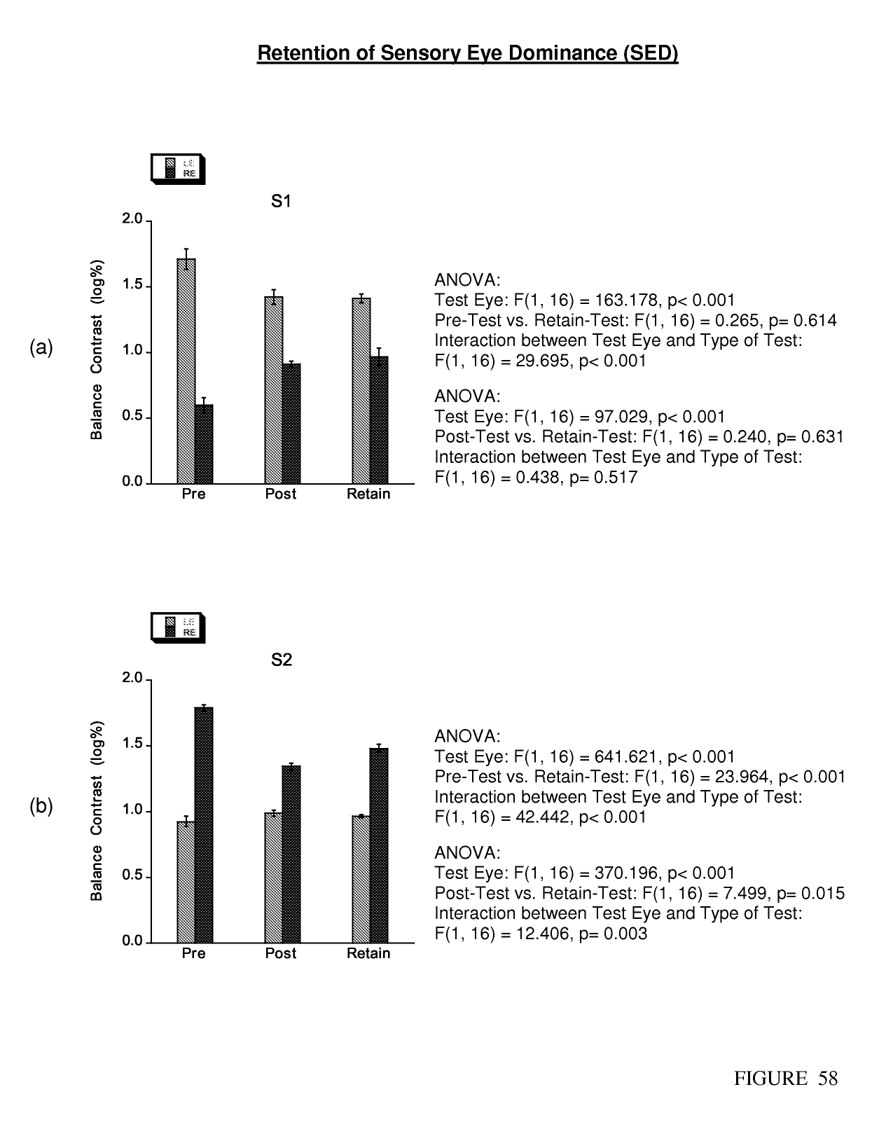

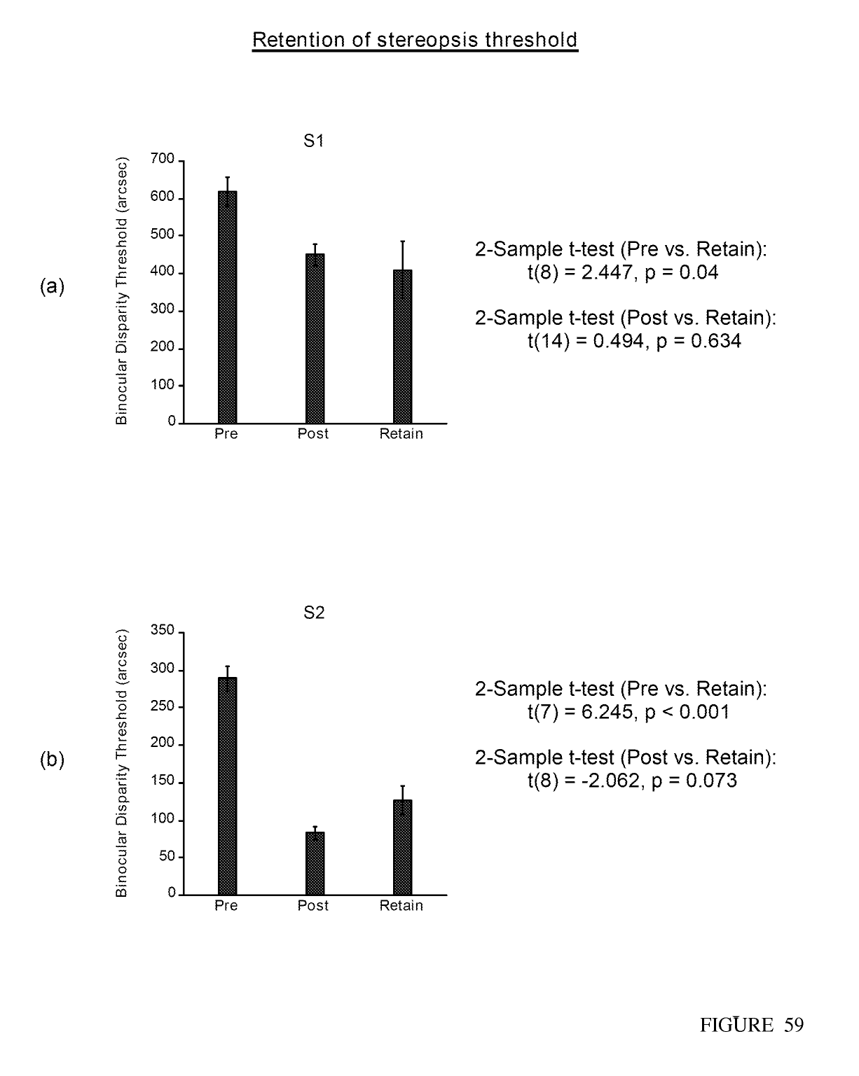

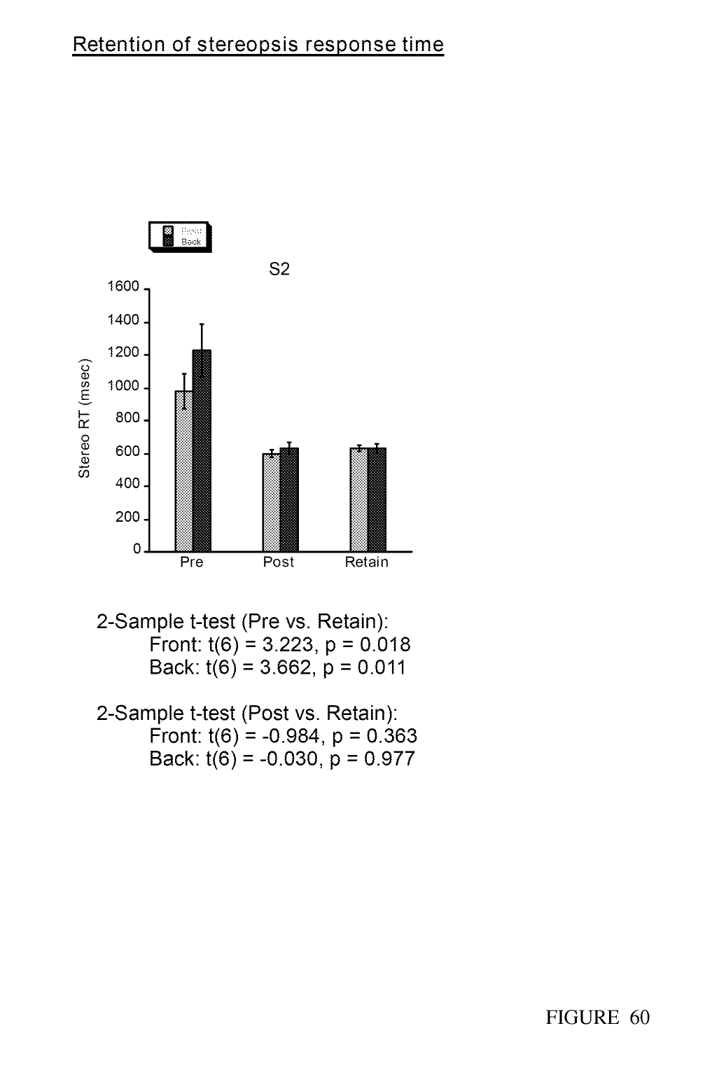

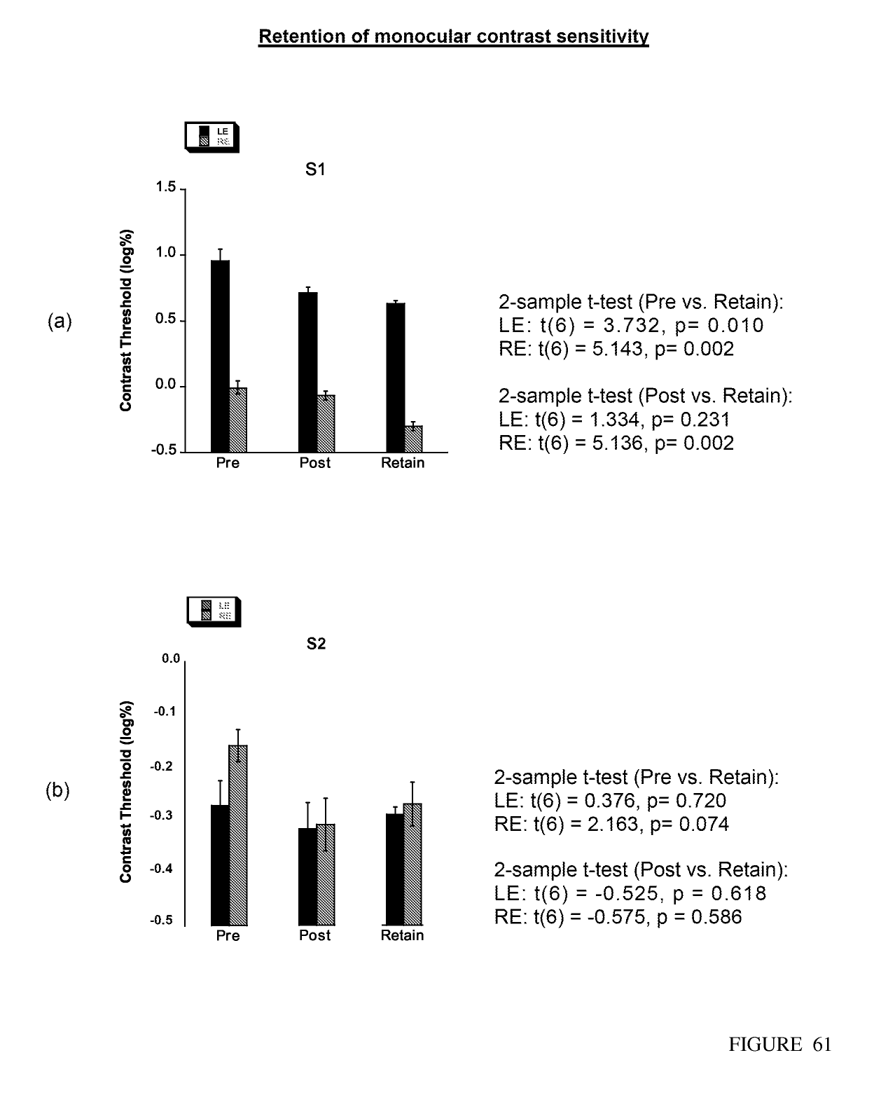

Observers S1 and S2 were retested for the retention of learned visual functions, respectively, at 5 and 3 months after the end of the training period. Learning was found to be largely retained in both observers for SED (FIG. 58), stereopsis (FIGS. 59 and 60) and monocular contrast sensitivity (FIG. 61). This demonstrates that the push-pull perceptual training has a long lasting effect.

What is claimed is:

1. A method for reducing sensory eye dominance or amblyopia in a subject comprising:
providing a first visual stimulus to at least a foveal visual region of a non-dominant eye of a subject and a second visual stimulus to at least a foveal region of a dominant eye of the subject, wherein visualization of the visual stimulus in the non-dominant eye is stimulated and visualization of the visual stimulus in the dominant eye is inhibited.

2. The method of claim 1, wherein visualization by the non-dominant eye is stimulated by presenting at least one attention cue to only the non-dominant eye, and optionally removing the attention cue, prior to presentation of the first and the second visual stimuli.

3. The method of claim 1, wherein the first and second visual stimuli are presented in the foveal visual region and one or more additional stimuli are presented in a parafoveal visual region of the subject.

4. The method of claim 1, wherein the first and second visual stimuli are a non-identical pair of dichoptic grating discs having the same or different contrast and/or mean luminance intensity.

5. The method of claim 4, wherein an orientation of the second visual stimulus is at an angle from 0 degree to 180 degrees, relative to an orientation of the first visual stimulus.

6. The method of claim 4, wherein the first visual stimulus is orthogonally oriented relative to the second visual stimulus.

7. The method of claim 4, wherein the dichoptic grating discs are provided against a grating background.

8. The method of claim 7, wherein the orientation of the first visual stimulus is at an angle from greater than 0 degrees to less than 180 degrees relative to the grating background.

9. The method of claim 7, wherein the second visual stimulus is approximately parallel to the grating background.

10. The method of claim 9, wherein the second visual stimulus is phase shifted from the grating background by an amount between about 0 degrees to about 180°.

11. A method for reducing sensory eye dominance or amblyopia in a subject comprising:
providing a first set of separate non-identical visual stimuli to a non-dominant eye and dominant eye of a subject, wherein visualization of the visual stimulus in the non-dominant eye is selected over visualization of the visual stimulus in the dominant eye;
removing the first set of separate non-identical visual stimuli; and
providing a second set of separate non-identical visual stimuli wherein visualization of the visual stimulus in the non-dominant eye is selected over visualization of the visual stimulus in the dominant eye, wherein at least one physical characteristic of the second set of visual stimulus presented to the non-dominant eye is different than that of the first set of visual stimuli.

12. The method of claim 11, wherein the physical characteristic is selected from the group consisting of stimulus orientation angle, stimulus contrast, an addition of one or more visual enhancement features, and combinations thereof.

13. The method of claim 12, wherein an orientation of the second visual stimulus presented to the non-dominant eye is at an angle of from greater than 0 degrees to less than 180 degrees, relative to an orientation of the first visual stimulus presented to the non-dominant eye.

14. The method of claim 12, wherein a contrast of the second visual stimulus presented to the non-dominant eye is higher or lower than the contrast of the first visual stimulus presented to the non-dominant eye.

15. The method of claim 12, wherein the visual stimuli provided to the non-dominant eye in the second set has a lower contrast than the visual stimuli provided to the non-dominant eye in the first set and/or the visual stimuli provided to the dominant eye in the second set has a higher contrast than the visual stimuli provided to the non-dominant eye in the first set.

16. The method of claim 12, wherein the visual stimuli presented to the non-dominant eye comprise at least one visual enhancement feature not provided to the dominant eye.

17. The method of claim 16, wherein the visual enhancement feature is selected from the group consisting of the addition of at least one contour ring, the addition of jitter, the addition of counterphase motion, the addition of mean luminance intensity, and combinations thereof.

18. A method for diagnosing sensory eye dominance in a subject with and without amblyopia comprising:
(a) providing a first visual stimulus to a non-dominant eye and a second visual stimulus to a dominant eye of a subject, wherein the first and second visual stimulus are non-identical;
(b) detecting which of the first visual stimulus and/or the second visual stimulus is predominantly visualized by the subject and which is predominantly not detected; and
(c) altering a physical characteristic of the visual stimulus that is predominantly not detected until a frequency of visualization of both the first and second stimuli is approximately the same.

19. The method of claim 18, wherein an orientation of the second visual stimulus is provided at an angle from about 0 degree to about 180 degrees, relative to an orientation of the first visual stimulus.

20. The method of claim 18, wherein the physical characteristic is contrast.

21. The method of claim 18, wherein the visual stimuli are provided in the subject's foveal region and/or parafoveal region.

22. The method of claim 18, wherein the mean luminance intensity of the first and second stimuli are different.

23. A method for establishing an sensory eye dominance profile in a subject with or without amblyopia comprising:
(a) providing a first visual stimulus to a first retinal location in a non-dominant eye and a second visual stimulus to a first retinal location in a dominant eye of a subject;
(b) detecting which of the first visual stimulus and/or the second visual stimulus is predominantly visualized by the subject and which is predominantly not detected;
(c) altering a physical characteristic of the visual stimulus that is predominantly not detected until a frequency of visualization of both the first and second stimuli is approximately the same; and
performing each of steps (a)-(c) in the foveal region and at successive concentric locations about the parafoveal region and peripheral retinal region.

24. A system for reducing sensory eye dominance or amblyopia in a subject comprising:
a visualization element adaptable to present a first visual stimulus to one eye of a subject and a second visual stimulus to a second eye of a subject;
a non-transient storage medium adaptable to present a first visual stimulus to a non-dominant eye of a subject and a second visual stimulus to a dominant eye of the subject such that visualization of the visual stimulus in the non-dominant eye of the subject is stimulated and visualization of the visual stimulus in the dominant eye is inhibited.
